(19) United States
(12) Patent Application Publication
(10) Pub. No.: US 2024/0200141 A1
(43) Pub. Date: Jun. 20, 2024

(54) COVALENT CHEMISTRY ENABLES EXTRACELLULAR VESICLE PURIFICATION ON NANOSUBSTRATES ? TOWARD EARLY DETECTION OF HEPATOCELLULAR CARCINOMA

(71) Applicant: The Regents of the University of California, Oakland, CA (US)

(72) Inventors: Hsian-Rong Tseng, Los Angeles, CA (US); Yazhen Zhu, Los Angeles, CA (US); Na Sun, Los Angeles, CA (US); Vatche G Agopian, Woodland Hills, CA (US)

(73) Assignee: The Regents of the University of California, Oakland, CA (US)

(21) Appl. No.: 17/915,071

(22) PCT Filed: Mar. 6, 2021
(86) PCT No.: PCT/US2021/024454

§ 371 Date: Sep. 27, 2022

Related U.S. Application Data
(60) Provisional application No. 63/000,776, filed on Mar. 27, 2020.

(51) Int. Cl. C12Q 1/6886; C12Q 1/6804; C12Q 1/6844; C12Q 1/6883

(52) U.S. Cl. CPC C12Q 1/6886; C12Q 1/6844; C12Q 1/6883; C12Q 1/6804; C12Q 2600/156; C12Q 2600/158

(57) ABSTRACT

Methods for selectively capturing an extracellular vesicle (EV) from a sample. including the steps of: functionalizing a capture agent for the EV with a first molecule from a first bioorthogonal functional group such that the capture agent remains attachable to the EV and the first molecule is also able to bond to a second molecule from a second bioorthogonal functional group. the second molecule being complementary to the first molecule: mixing the functionalized capture agent with the sample such that the functionalized capture agent binds to the EV and such that an activated sample is formed: functionalizing a capture surface with the second molecule: and depositing at least a portion of the activated sample on at least a portion of the functionalized capture surface to thereby selectively capture the EV by binding of the second molecule with the first molecule.

### CROSS-REFERENCE OF RELATED APPLICATIONS

This application is a U.S. National Stage of PCT/US2021/024454, filed Mar. 26, 2021, which claims priority to U.S. Provisional Application No. 63/000,776 filed Mar. 27, 2020; the entire contents of which are hereby incorporated by reference.

### GOVERNMENT SUPPORT

This invention was made with government support under Grant Number CA198900 and CA235340, awarded by the National Institutes of Health. The government has certain rights in the invention.

### BACKGROUND

### 1. Technical Field

Aspects of the invention relate to compositions, systems and methods for capturing extracellular vesicles, and more particularly to bioorthogonal ligation mediated extracellular vesicle capture.

### 2. Discussion of Related Art

Extracellular vesicles (EVs),^{[1,2]} as a heterogeneous group of phospholipid bilayer-enclosed particles, can be released by all types of cells, especially tumor cells. Recently, the scientific community has begun to understand the importance of EVs as a mechanism and vehicle^{[3]} of cellular interchange of bioactive molecules,^{[4]} including proteins, DNA, and RNA.^{[5,6]} Such interchanges can result in exchanges of genetic information and functional molecules,^{[7]} leading to the subsequent reprogramming of the recipient cells.^{[8-10]} Tumor-derived EVs are regarded as “biological shuttles”^{[11]} capable of transporting biomolecules to mediate intracellular communication, microenvironment modulation, and cancer metastasis. Therefore, in addition to exploring the diagnostic values of tumor-derived EVs,^{[12-14]} there is growing interest in performing functional studies of tumor-derived EVs in cellular communication,^{[15,16]} (e.g., EV uptake and cargo transfer). Since tumor-derived EVs exist in a background of non-tumor-derived EVs, selectively purification of tumor-derived EVs—while retaining the integrity of their enclosed biomolecular cargos—has been identified as a major technical barrier to conducting the functional studies of tumor-derived EVs.

Conventionally, EVs can be isolated from blood plasma or serum based on their physical properties by using enrichment methods, e.g., ultracentrifugation,^{[17]} precipitation,^{[18]} filtrations,^{[19]} size-based microfluidics,^{[20-22]} and lipid-based nanoprobes.^{[23]} However, these approaches are not suitable for specifically enriching tumor-derived EVs from EVs in the background. Significant research endeavors^{[24,25]} have been devoted to exploring antibody or aptamer^{[26,27]}-based techniques^{[28-30]} to enrich and analyze tumor-derived EVs. For instance, GPC1 antibody-coated beads have been used to isolate pancreatic cancer-derived exosomes;^{[31]} a herringbone microfluidic device (a.k.a. ^{EV}HB-Chip) functionalized with EGFRvIII antibody has been demonstrated to enrich glioblastoma-derived exosomes.^{[32]} Previously, a “Nano Villi Chip”.^{[33]} was developed in which densely packed, anti-epithelial cell adhesion molecule (anti-EpCAM)-grafted silicon (Si) nanowire arrays were engineered to achieve efficient and reproducible immune-affinity capture of tumor-derived EVs. However, due to the limited number of antigens present on the surface of individual EVs, immune-affinity EV capture approaches, which are driven by the dynamic binding between a pair of antigens (on EVs) and antibodies (on the substrates), often suffer from poor EV capture performance and high background. Moreover, conducting functional studies of tumor-derived EVs requires the purification of tumor-derived EVs with biological intactness. Therefore, it is necessary to develop novel purification systems with the capacity for both sensitive and specific capture of tumor-derived EVs and their subsequent release.

### INCORPORATION BY REFERENCE

All publications and patent applications identified herein are incorporated by reference in their entirety and to the same extent as if each individual publication or patent application was specifically and individually indicated to be incorporated by reference.

### SUMMARY

An embodiment of the invention relates to a method of selectively capturing an extracellular vesicle (EV) from a sample, including: functionalizing a capture agent for the EV with a first molecule from a first bioorthogonal functional group such that the capture agent remains attachable to the EV and the first molecule is also able to bond to a second molecule from a second bioorthogonal functional group, the second molecule being complementary to the first molecule; mixing the functionalized capture agent with the sample such that the functionalized capture agent binds to the EV and such that an activated sample is formed; functionalizing a capture surface with the second molecule; and depositing at least a portion of the activated sample on at least a portion of the functionalized capture surface to thereby selectively capture the EV by binding of the second molecule with the first molecule. In such an embodiment, the first molecule from the first bioorthogonal functional group and the capture agent are present in a molar ratio of between 2:1 to 10:1.

An embodiment of the invention relates to a method of assaying for a cancer in a subject including: selectively capturing an extracellular vesicle (EV) from a sample from the subject, the selectively capturing an EV including: functionalizing a capture agent for the EV with a first molecule from a first bioorthogonal functional group such that the capture agent remains attachable to the EV and the first molecule is also able to bond to a second molecule from a second bioorthogonal functional group, the second molecule being complementary to the first molecule; mixing the functionalized capture agent with the sample such that the functionalized capture agent binds to the EV and such an activated sample is formed; functionalizing a capture surface with the second molecule; and depositing at least a portion of the activated sample on at least a portion of the functionalized capture surface to thereby selectively capture the EV by binding of the molecule with the first molecule; releasing the EV from the capture surface; assaying a nucleic acid sequence from the EV; and determining from the assaying of the nucleic acid sequence from the EV whether the cancer is present in the subject. In such an embodiment, the first molecule from the first bioorthogonal functional group and the capture agent are present in a molar ratio of between 2:1 to 10:1.

An embodiment of the invention relates to a method of assaying for a cancer in a subject including: selectively capturing a plurality of extracellular vesicles (EVs) from a sample from the subject, where each extracellular vesicle (EV) of the plurality of EVs is selectively captured including: functionalizing a capture agent for an EV of the plurality of EVs with a first molecule from a first bioorthogonal functional group such that the capture agent remains attachable to the EV and the first molecule is also able to bond to a second molecule from a second bioorthogonal functional group, the second molecule being complementary to the first molecule; mixing the functionalized capture agent with the sample such that the functionalized capture agent binds to the EV and such an activated sample is formed; functionalizing a capture surface with the second molecule; and depositing at least a portion of the activated sample on at least a portion of the functionalized capture surface to thereby selectively capture the EV by binding of the molecule with the first molecule; releasing the plurality of EVs from the capture surface; assaying a plurality of nucleic acid sequences from the plurality of EVs; creating an expression profile of the plurality of nucleic acid sequences, the expression profile including a quantification of each of the plurality of nucleic acid sequences; comparing the expression profile with a control; and determining from the comparing of the expression profile with the control whether the cancer is present in the subject. In such an embodiment, the first molecule from the first bioorthogonal functional group and the capture agent are present in a molar ratio of between 2:1 to 10:1.

An embodiment of the invention relates to a kit for selectively capturing an extracellular vesicle (EV) from a sample, the kit including: a capture agent having a first molecule from a first bioorthogonal functional group; a substrate having a functionalized capture surface having a second molecule from a second bioorthogonal functional group, the second molecule being complementary to the first molecule; a cleaving agent; instructions for mixing the capture agent with the sample such that the capture agent binds to the EV and such that an activated sample is formed; instructions for depositing at least a portion of the activated sample on at least a portion of the functionalized capture surface to thereby selectively capture the EV by binding of the second molecule with the first molecule; and instructions for using the cleaving agent to release the EV from the capture surface. In such an embodiment, the first molecule from the first bioorthogonal functional group and the capture agent are present in a molar ratio of between 2:1 to 10:1.

### BRIEF DESCRIPTION OF THE DRAWINGS

FIGS. 1A and 1B show Extra-Cellular (EV) Click Chips for purification and molecular characterization of hepatocellular carcinoma extracellular vesicles (HCC EVs) according to an embodiment.

FIGS. 2A-2J are data graphs showing results of optimization of EV Click Chips using artificial plasma samples according to an embodiment.

FIGS. 3A-3E are illustrations and microscopic images showing the characterization of HepG2 EVs purified by EV Click Chips according to an embodiment.

FIGS. 4A and 4B show results for RT-ddPCR assay for quantification of 10 HCC-specific mRNA transcripts in HCC EVs purified by EV Click Chips according to an embodiment.

FIGS. 5A-5D are data graphs showing statistical analysis on HCC EVZ Scores in different cohorts according to an embodiment.

FIG. 6 is a schematic summary of the stepwise functional group transformation employed for the preparation of Tz-grafted SiNWS according to an embodiment.

FIGS. 7A and 7B are data graphs showing results for antibody and antibody cocktail optimization and selection according to an embodiment.

FIGS. 8A-8D are schematics showing various Click Chemistry motifs according to embodiments of the invention.

FIG. 9 is a schematic illustration of “ES-EV Click Chip” and its working mechanism based on covalent chemistry-mediated purification of ES EVs according to an embodiment.

FIGS. 10A-10D are microscopic images and graphs showing detection of LINGO-1 expression on ES cell lines and ES EVs according to an embodiment of the invention. are microscopic images, bar graphs, and schematics showing characterization of ES EVs before and after the capture/release on ES-EV Click Chips according to an embodiment.

FIGS. 11A-11H are microscopic images and schematics showing characterization of the ES-EV capture and release process according to an embodiment of the invention.

FIGS. 12A-12K are schematics and graphs showing evaluation and optimization of EV-capture/release performance of ES-EV Click Chips according to an embodiment of the invention.

FIGS. 13A-13E are schematics and graphs showing detection of EWS rearrangements in ES EVs by coupling ES-EV Click Chips with RT-ddPCR according to an embodiment of the invention.

FIGS. 14A-14F are schematics, graphs, and microscopic images showing results of downstream functional studies using the ES EVs purified by ES-EV Click Chips according to an embodiment of the invention.

FIGS. 15A and 15B are schematics showing use of EV Click Chips for purification and molecular characterization of prostate cancer extracellular vesicles (PCa EVs) according to an embodiment.

FIGS. 16A-16E are schematics and graphs showing optimization of EV Click Chips for capture of PCa-derived EVs using artificial plasma samples according to an embodiment.

FIGS. 17A and 17B show results of a RT-ddPCR assay for quantification of 6 PCa-specific mRNA transcripts in PCa EVs purified by EV Click Chips according to an embodiment.

FIG. 18 is a schematic showing use of EV Click Chips for purification and molecular characterization of Placenta-derived EVs for placenta disease detection according to an embodiment.

FIGS. 19A-19F are graphs showing optimization of EV Click Chips for capture of placenta-derived EVs using artificial plasma samples according to an embodiment.

FIGS. 20A-20D are a schematic overview of rapid isolation and analysis system for EVs from patient blood plasma samples according to an embodiment.

FIGS. 21A-21D are schematics and graphs showing surface modification and characterization of Tz-grafted silica microbeads (silica MBs) according to an embodiment.

FIGS. 22A-22F are images and graphs showing the characterization of tumor-derived EVs in solution and captured on silica MBs according to an embodiment.

FIGS. 23A-23I are graphs showing optimization of isolation efficiency for total EVs (β-actin) and A673 EVs (EWS/Fli-1) using artificial blood plasma samples according to an embodiment.

FIGS. 24A-24C are schematics showing a rapid isolation and analysis system for placenta-derived EVs from maternal blood plasma samples according to an embodiment.

FIGS. 25A and 25B are graphs showing optimization of Click Beads for isolation of placenta-derived EVs using maternal plasma samples and female healthy donor plasma samples according to an embodiment.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification, including the Background and Detailed Description sections, are incorporated by reference as if each had been individually incorporated.

The term “bioorthogonal chemistry” refers to any chemical reaction that can occur inside of living systems without interfering with native biochemical processes.

The term capture agent can include any molecules, particles, etc. that selectively bind to particular rare cells such as, but not limited to antibodies.

A non-limiting example of a chemical reaction for use according to embodiments of the invention is the 1,3-dipolar cycloaddition between azides and cyclooctynes as described in the scheme below:

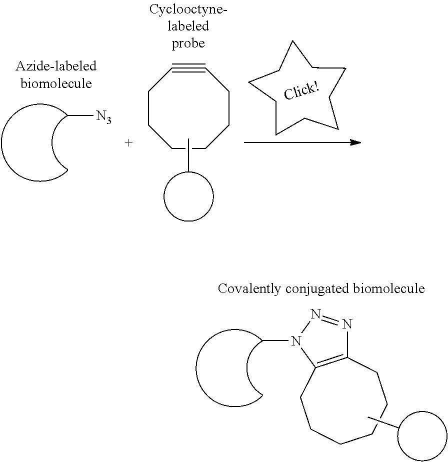

A non-limiting example of a chemical reaction for use according to embodiments of the invention is Nitrone Dipole Cycloaddition as described in the scheme below:

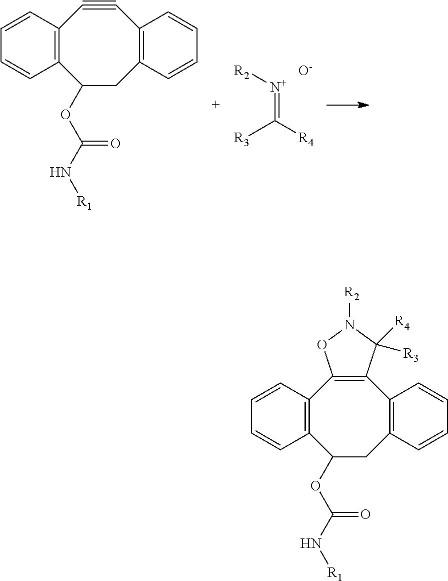

This cycloaddition between a nitrone and a cyclooctyne forms N-alkylated isoxazolines.

A non-limiting example of a chemical reaction for use according to embodiments of the invention is Norbornene Cycloaddition as described in the scheme below:

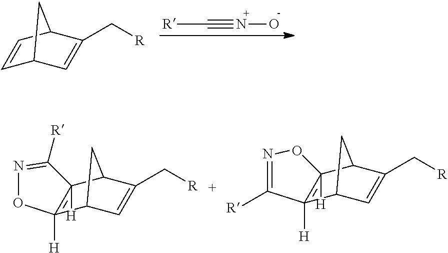

Nitrile oxide as a 1,3-dipole and a norbornene as a dipolarophile

A non-limiting example of a chemical reaction for use according to embodiments of the invention is Oxanorbornadiene Cycloaddition as described in the scheme below:

The oxanorbornadiene cycloaddition is a 1,3-dipolar cycloaddition followed by a retro-Diels Alder reaction to generate a triazole-linked conjugate with the elimination of a furan molecule.

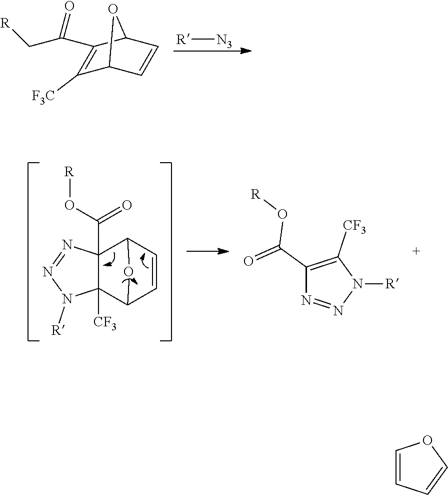

A non-limiting example of a chemical reaction for use according to embodiments of the invention is Tetrazine Ligation as described in the scheme below:

The reaction of a trans-cyclooctene and an s-tetrazine in an inverse-demand Diels Alder reaction followed by a retro-Diels Alder reaction to eliminate nitrogen gas.

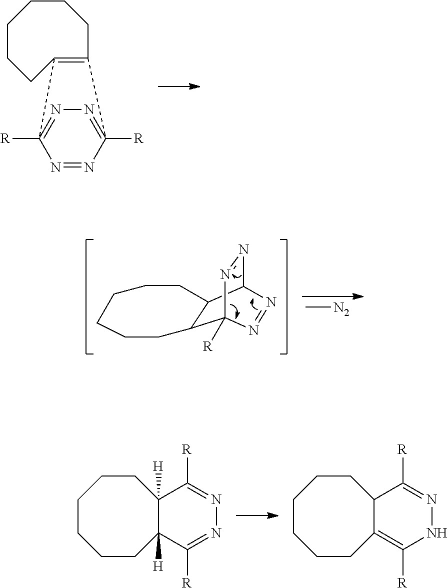

A non-limiting example of a chemical reaction for use according to embodiments of the invention is [4+1] Cycloaddition as described in the scheme below:

This isocyanide click reaction is a [4+1] cycloaddition followed by a retro-Diels Alder elimination of N₂.

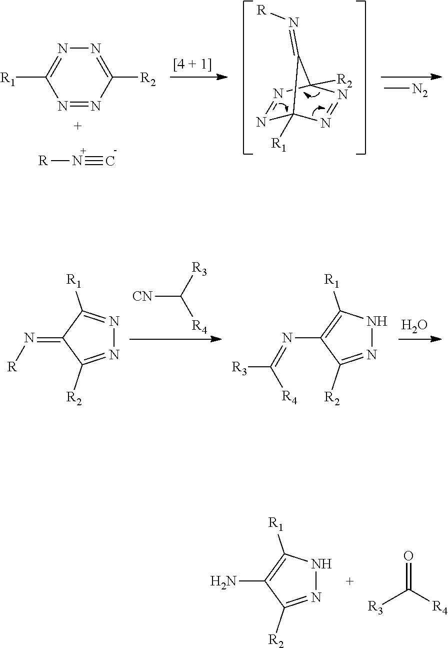

A non-limiting example of a chemical reaction for use according to embodiments of the invention is Quadricyclane Ligation as described in the scheme below:

Photoclick chemistry utilizes a photoinduced cycloreversion to release N₂.

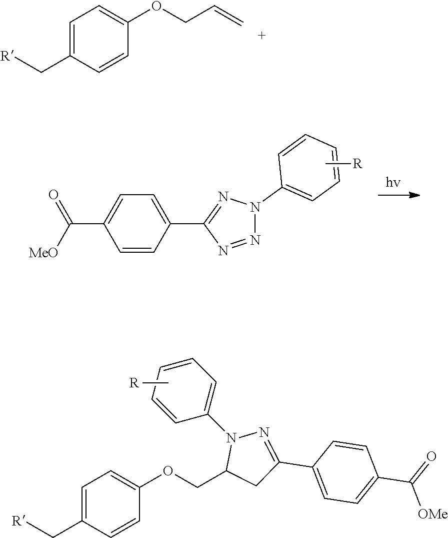

A non-limiting example of a chemical reaction for use according to embodiments of the invention is Quadricyclane Ligation as described below:

The quadricyclane ligation utilizes a highly strained quadricyclane to undergo [2+2+2] cycloaddition with π systems.

Some embodiments of the invention include a device for capturing an extracellular vesicle. Examples of such devices are described in U.S. Pat. No. 9,140,697 which is hereby incorporated by references in its entirety. A further non-limiting example of such a device is a Silicon Nanowire Substrates (SiNWS). In embodiments of the invention, the device includes a substrate; and a plurality of nanowires at least one of attached to or integral with a surface of the substrate such that each nanowire of the plurality of nanowires has an unattached end.

In some embodiment. the device for capturing a cell includes a substrate having a nanostructured surface region. Also, in some embodiments, a plurality of binding agents are attached to the nanostructured surface region of the substrate. However, binding agents are not required for the device to bind to target extracellular vesicles. The nanostructured surface region includes a plurality of nanostructures, each having a longitudinal dimension and a lateral dimension. As a sample is placed on the device, extracellular vesicles are selectively captured by the binding agents and the plurality of nanostructures acting in cooperation (in embodiments having binding agents). When present, the binding agent or agents employed will depend on the type of extracellular vesicles being targeted. Conventional binding agents are suitable for use in some of the embodiments of the present invention. Non-limiting examples of binding agents include antibodies, nucleic acids, oligo- or polypeptides, cellular receptors, ligands, aptamers, biotin, avidin. Coordination complexes, synthetic polymers, and carbohydrates. In some embodiments of the present invention, binding agents are attached to the nanostructured surface region using conventional methods. The method employed will depend on the binding agents and the material used to construct the device. Non-limiting examples of attachment methods include non-specific adsorption to the surface, either of the binding agents or a compound to which the agent is attached or chemical binding, e.g., through self-assembled monolayers or silane chemistry. In some embodiments, the nanostructured surface region is coated with streptavidin and the binding agents are biotinylated, which facilitates attachment to the nanostructured surface region via interactions with the streptavidin molecules.

In some embodiments of the present invention, the nanostructures increase the surface area of the substrate and increase the probability that a given extracellular vesicle will come into contact. In these embodiments, the nanostructures can enhance binding of the target extracellular vesicles by interacting with surface components. In some embodiments, the nanostructures have a longitudinal dimension that is equal to its lateral dimension, where both the lateral dimension and the longitudinal dimension is less than 1 mm, i.e., nanoscale in size. In other embodiments, the nanostructures have a longitudinal dimension that is at least ten times greater than its lateral dimension. In further embodiments, the nanostructures have a longitudinal dimension that is at least twenty times greater, fifty times greater, or 100 times greater than its lateral dimension. In some embodiments, the lateral dimension is less than 1 mm. In other embodiments, the lateral dimension is between 1-500 nm. In further embodiments, the lateral dimension is between 30-400 nm. In still further embodiments, the lateral dimension is between 50-250 nm. In some embodiments, the longitudinal dimension is at least 1 mm long. In other embodiments, the longitudinal dimension is between 1-50 mm long. In other embodiments, the longitudinal dimension is 1-25 mm long. In further embodiments, the longitudinal dimension is 5-10 mm long. In still further embodiments, the longitudinal dimension is at least 6 mm long. The shape of the nanostructure is not critical. In some embodiments of the present invention, the nanostructure is a sphere or a bead. In other embodiments, the nanostructure is a strand, a wire, or a tube. In further embodiments, a plurality of nanostructure contains one or more of nanowires, nanofibers, nanotubes, nano-pillars, nanospheres, or nanoparticles.

An embodiment of the invention relates to a method of selectively capturing an extracellular vesicle (EV) from a sample, including: functionalizing a capture agent for the EV with a first molecule from a first bioorthogonal functional group such that the capture agent remains attachable to the EV and the first molecule is also able to bond to a second molecule from a second bioorthogonal functional group, the second molecule being complementary to the first molecule; mixing the functionalized capture agent with the sample such that the functionalized capture agent binds to the EV and such that an activated sample is formed; functionalizing a capture surface with the second molecule; and depositing at least a portion of the activated sample on at least a portion of the functionalized capture surface to thereby selectively capture the EV by binding of the second molecule with the first molecule. In such an embodiment, the first molecule from the first bioorthogonal functional group and the capture agent are present in a molar ratio of between 2:1 to 10:1.

An embodiment of the invention relates to the method above, where the first molecule from the biorthogonal functional group is selected from the list consisting of trans-cyclooctene (TCO), Alkyne, and a cyclooctyne derivative.

An embodiment of the invention relates to the method above, where the cyclooctyne derivative includes dibenzylcyclooctyne (DBCO) or biarylazacyclooctynone (BARAC).

An embodiment of the invention relates to the method above, where the second molecule from the second biorthogonal functional group is selected from the list consisting of tetrazine (TZ) and azide.

An embodiment of the invention relates to the method above, where the capture surface includes a nanostructured surface.

An embodiment of the invention relates to the method above, further including functionalizing a second capture agent for the EV with the first molecule such that the second capture agent remains attachable to the EV and the first molecule is also able to bond to the molecule including the second molecule from the second bioorthogonal functional group, and where the second capture agent is distinct from the capture agent.

An embodiment of the invention relates to a method of assaying for a cancer in a subject including: selectively capturing an extracellular vesicle (EV) from a sample from the subject, the selectively capturing an EV including: functionalizing a capture agent for the EV with a first molecule from a first bioorthogonal functional group such that the capture agent remains attachable to the EV and the first molecule is also able to bond to a second molecule from a second bioorthogonal functional group, the second molecule being complementary to the first molecule; mixing the functionalized capture agent with the sample such that the functionalized capture agent binds to the EV and such an activated sample is formed; functionalizing a capture surface with the second molecule; and depositing at least a portion of the activated sample on at least a portion of the functionalized capture surface to thereby selectively capture the EV by binding of the molecule with the first molecule; releasing the EV from the capture surface; assaying a nucleic acid sequence from the EV; and determining from the assaying of the nucleic acid sequence from the EV whether the cancer is present in the subject. In such an embodiment, the first molecule from the first bioorthogonal functional group and the capture agent are present in a molar ratio of between 2:1 to 10:1.

An embodiment of the invention relates to the method above, where the first molecule from the biorthogonal functional group is selected from the list consisting of trans-cyclooctene (TCO), Alkyne, and a cyclooctyne derivative.

An embodiment of the invention relates to the method above, where the cyclooctyne derivative includes dibenzylcyclooctyne (DBCO) or biarylazacyclooctynone (BARAC).

An embodiment of the invention relates to the method above, where the second molecule from the second biorthogonal functional group is selected from the list consisting of tetrazine (TZ) and azide.

An embodiment of the invention relates to the method above, where the capture surface includes a nanostructured surface.

An embodiment of the invention relates to the method above, further including functionalizing a second capture agent for the EV with the first molecule such that the second capture agent remains attachable to the EV and the first molecule is also able to bond to the molecule including the second molecule from the second bioorthogonal functional group, and where the second capture agent is distinct from the capture agent.

An embodiment of the invention relates to the method above, where the releasing the EV from the capture surface includes use of a cleaving agent.

An embodiment of the invention relates to a method of assaying for a cancer in a subject including: selectively capturing a plurality of extracellular vesicles (EVs) from a sample from the subject, where each extracellular vesicle (EV) of the plurality of EVs is selectively captured including: functionalizing a capture agent for an EV of the plurality of EVs with a first molecule from a first bioorthogonal functional group such that the capture agent remains attachable to the EV and the first molecule is also able to bond to a second molecule from a second bioorthogonal functional group, the second molecule being complementary to the first molecule; mixing the functionalized capture agent with the sample such that the functionalized capture agent binds to the EV and such an activated sample is formed; functionalizing a capture surface with the second molecule; and depositing at least a portion of the activated sample on at least a portion of the functionalized capture surface to thereby selectively capture the EV by binding of the molecule with the first molecule; releasing the plurality of EVs from the capture surface; assaying a plurality of nucleic acid sequences from the plurality of EVs; creating an expression profile of the plurality of nucleic acid sequences, the expression profile including a quantification of each of the plurality of nucleic acid sequences; comparing the expression profile with a control; and determining from the comparing of the expression profile with the control whether the cancer is present in the subject. In such an embodiment, the first molecule from the first bioorthogonal functional group and the capture agent are present in a molar ratio of between 2:1 to 10:1.

An embodiment of the invention relates to a kit for selectively capturing an extracellular vesicle (EV) from a sample, the kit including: a capture agent having a first molecule from a first bioorthogonal functional group; a substrate having a functionalized capture surface having a second molecule from a second bioorthogonal functional group, the second molecule being complementary to the first molecule; a cleaving agent; instructions for mixing the capture agent with the sample such that the capture agent binds to the EV and such that an activated sample is formed; instructions for depositing at least a portion of the activated sample on at least a portion of the functionalized capture surface to thereby selectively capture the EV by binding of the second molecule with the first molecule; and instructions for using the cleaving agent to release the EV from the capture surface. In such an embodiment, the first molecule from the first bioorthogonal functional group and the capture agent are present in a molar ratio of between 2:1 to 10:1.

An embodiment of the invention relates to the kit above, where the first molecule from the biorthogonal functional group is selected from the list consisting of trans-cyclooctene (TCO), Alkyne, and a cyclooctyne derivative.

An embodiment of the invention relates to the kit above, where the cyclooctyne derivative includes dibenzylcyclooctyne (DBCO) or biarylazacyclooctynone (BARAC).

An embodiment of the invention relates to the kit above, where the second molecule from the second biorthogonal functional group is selected from the list consisting of tetrazine (TZ) and azide.

An embodiment of the invention relates to the kit above, where the capture surface includes a nanostructured surface.

An embodiment of the invention relates to the kit above, further including a plurality of reagents for a nucleic acid test.

The embodiments illustrated and discussed in this specification are intended only to teach those skilled in the art how to make and use the invention. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

### EXAMPLES

### Example 1

Hepatocellular carcinoma (HCC) is the fourth most common cause of cancer-related deaths worldwide¹. The poor prognosis of HCC can be attributed to the fact that diagnosis is often made at a late stage in disease development^{2,3}. Earlier detection of HCC is critical to reducing the high HCC mortality rates, as numerous potentially curative therapeutic interventions are available to treat early-stage HCC. Current American Association for the Study of Liver Disease (AASLD) guidelines³ recommend biannual liver ultrasonography with or without serum alpha-fetoprotein (AFP) for at-risk patients with cirrhosis and chronic liver disease; however, ultrasound is not sensitive enough to detect early lesions, and the reported performance of AFP varies widely⁴. Thus, the development of novel non-invasive diagnostics for early-stage HCC may significantly benefit at-risk patients at risk.

Among the three conventional liquid biopsy^{5,6} approaches in the context of oncology, i.e., circulating tumor cells (CTCs)⁷⁻⁹, circulating tumor DNA (ctDNA)^{10,11}, and extracellular vesicles (EVs)¹², EVs are present in circulation at relatively early stages of disease¹³ and persist across all disease stages. Furthermore, EVs' inherent stability guarantees the integrity of encapsulated biomolecular cargos, especially the extremely fragile mRNA. Therefore, tumor-derived EVs are regarded as “biomarker reservoirs”¹⁴, promising the implementation of downstream molecular analysis for non-invasive cancer diagnosis¹⁵. However, the conventional EV isolation methods, e.g., ultracentrifugation¹⁶ and precipitation processing, are based on EVs' physical properties (most notably density and solubility), which are incapable of separating tumor-derived EVs from total EVs. Since the majority of EVs in circulation are not of tumor origin, analyzing total EVs is of limited diagnostic power as a result of high background noise¹⁷. To overcome this issue, groups^{17,18,19,20} have developed various immunoaffinity-based approaches to enrich tumor-derived EVs. Evidence is emerging that EVs and their biomolecular cargos such as RNA have the potential to detect HCC²¹. Nonetheless, exploring the use of HCC EV-derived mRNA signatures as biomarkers for detecting HCC, especially early-stage HCC from at-risk chronic liver diseases (e.g., hepatitis and liver cirrhosis) is still limited by a number of challenges, including i) developing an EV purification system that can accommodate a multimarker cocktail to recognize, enrich, and recover HCC EVs secreted from the highly heterogeneous HCC²²⁻²⁴, ii) avoiding mRNA degradation by streamlining the EV purification process, and iii) seamlessly coupling a simple and quantitative downstream molecular assay with HCC EV purification systems.

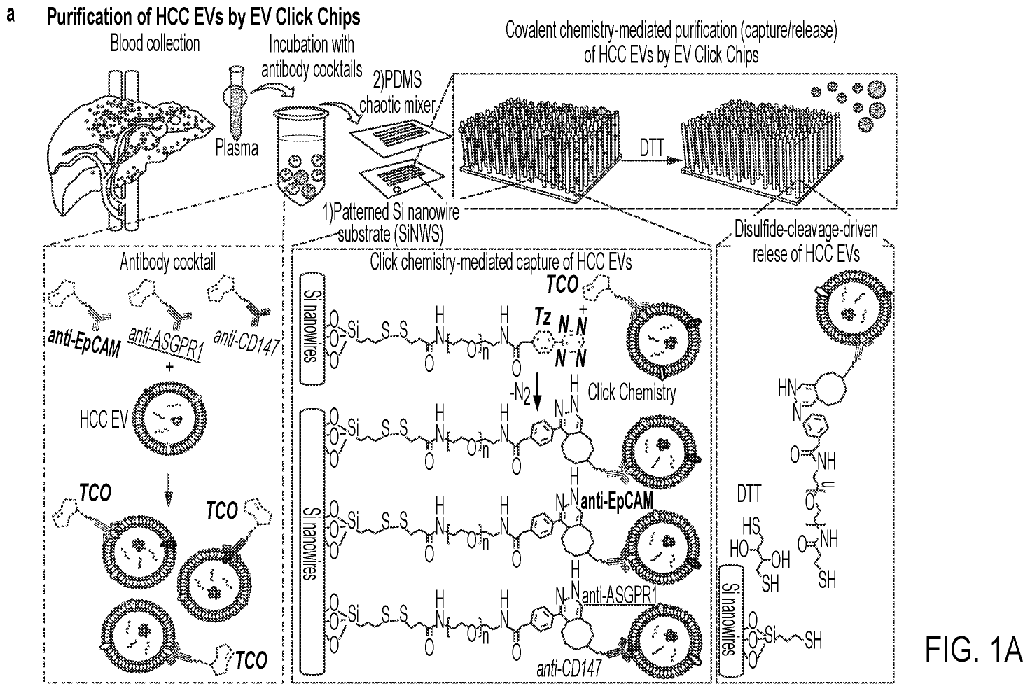

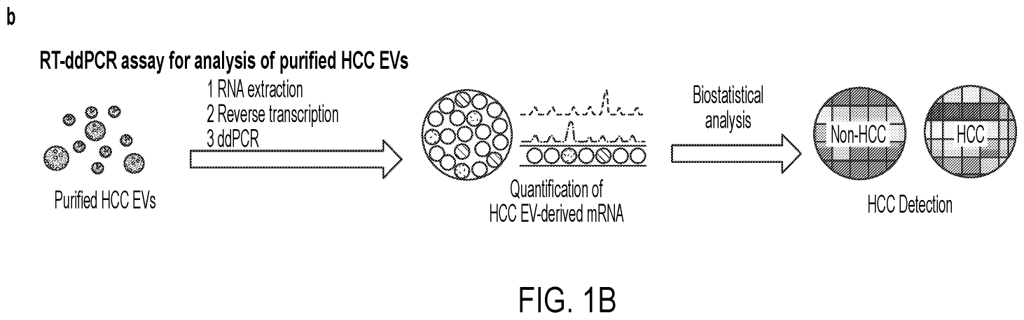

The poor prognosis of hepatocellular carcinoma (HCC) is due to the fact that the majority of patients present with advanced stage disease. Among different tumor liquid biopsy approaches, extracellular vesicles (EVs) are present in circulation at relatively early stage of disease, thus opening up noninvasive diagnostic opportunity for HCC early detection. Here, a new HCC EV purification system (i.e., EV Click Chips) is described (FIGS. 1A and 1B). The system described synergistically integrates four very powerful approaches, including covalent chemistry-mediated EV capture/release, multimarker antibody cocktail²⁵, nanostructured substrates²⁶, and a microfluidic chaotic mixer²⁷, paving the way for implementation of non-invasive detection of early-stage HCC.

The covalent chemistry-mediated EV capture/release was built upon the combined use of click chemistry²⁸-mediated EV capture and disulfide cleavage²⁹-driven EV release in conjunction with an optimized multi-marker cocktail targeting three HCC-associated surface markers²⁵, including EpCAM, ASGPR, and CD147. Further, the incorporation of densely packed silicon nanowires substrates (SiNWS) dramatically increases the device surface area²⁶ contacting/interacting with EVs. Moreover, the microfluidic chaotic mixer facilitates repeated physical contact³⁰ between SiNWS and the flow-through HCC EVs, further enhancing the performance of EV capture. In contrast to previous antibody-mediated EV capture¹⁸, a pair of highly reactive click chemistry motifs³¹ i.e., tetrazine (Tz) and trans-cyclooctene (TCO), were grafted onto EV capture substrates (i.e., SiNWS, via surface modification) and HCC EVs (via TCO-capture agent conjugation), respectively. The click chemistry reaction between Tz-grafted SiNWS and TCO-grafted HCC EVs is rapid specific, irreversible, and bioorthogonal³¹, resulting in immobilization of the HCC EVs with improved capture efficiency and reduced background. After click chemistry-mediated HCC EV capture, exposure to a disulfide cleavage agent, 1,4-dithiothreitol (DTT)³² leads to the prompt release of the HCC EVs from the SiNWS by cleaving the disulfide bond linking the Tz to the SiNWS. Recognizing that the field has been in dire need of practical methods capable of quantitatively assessing the performance (EV recovery yield and purity) of any given EV purification system, a quantitative evaluation method for assessing the performance of EV Click Chip is also described. By adopting this quantitative method throughout the optimization process, one is able to accurately determine the performance of EV Click Chips, achieving an optimal HCC EV purification condition that was used in pre-clinical studies.

The potential of a streamlined HCC EV-based mRNA assay was exploited by coupling i) EV Click Chips for purification of HCC EVs and ii) reverse-transcription droplet digital PCR (RT-ddPCR) for quantification of 10 well-validated HCC-specific mRNA transcripts³³ using plasma samples of HCC patients and control cohorts. After conducting biostatistical analysis, HCC EV-derived 10-gene digital readouts exhibited a great potential for non-invasive early detection of HCC from at-risk cirrhotic patients.

FIGS. 1A and 1B show Extra-Cellular (EV) Click Chips for purification and molecular characterization of hepatocellular carcinoma extracellular vesicles (HCC EVs) according to an embodiment. FIG. 1A is a schematic illustration of the device configuration and work mechanism of an EV Click Chip, which is composed of a patterned Si nanowire substrate (SiNWS) covalently functionalized with tetrazine (Tz), and an overlaid polydimethylsiloxane (PDMS) chaotic mixer. The covalent chemistry-mediated EV purification approach combines the click chemistry-mediated EV capture and disulfide cleavage-driven EV release in conjunction with the use of an antibody cocktail targeting three HCC-associated surface markers, i.e., EpCAM, ASGPR, and CD147. A pair of highly reactive click chemistry motifs, i.e., Tz and trans-cyclooctene (TCO), are grafted onto SiNWS and EVs, respectively. When a plasma sample flows through the device, click chemistry reaction between Tz-grafted SiNWS and TCO-grafted HCC EVs results in the immobilization of the HCC EVs. Subsequently, the exposure to 1,4-dithiothreitol (DTT) leads to the cleavage of the embedded disulfide bonds to release the immobilized HCC EVs. FIG. 1B shows that the purified HCC EVs can then be subjected to reverse-transcription droplet digital PCR (RT-ddPCR) to obtain the digital readouts of 10 HCC-specific genes, which can be used to distinguish HCC patients from at-risk cirrhotic patients.

### Results

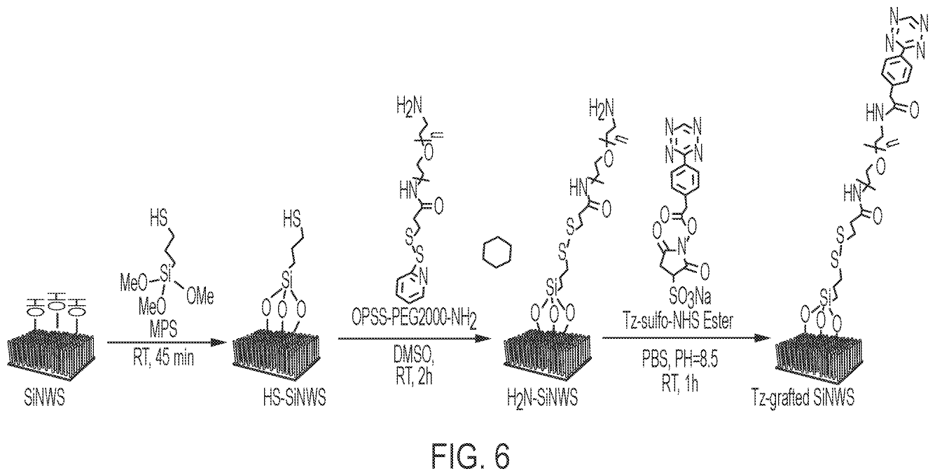

The design and preparation of an EV Click Chip. An EV Click Chip (as seen in FIGS. 1A and 1B) is composed of two functional components: (i) a patterned Si nanowire substrate (SiNWS)²⁶ covalently functionalized with disulfide bonds that link to terminal Tz motifs and (ii) an overlaid polydimethylsiloxane (PDMS) chaotic mixer³⁴, housed in a custom-designed microfluidic chip holder. 10-15 μm densely packed Si nanowires (diameter=100-200 nm) were introduced onto SiNWS, thus offering approximately 30 times more surface area (in contrast to a flat substrate) for facilitating click chemistry-mediated EV capture. The incorporation of disulfide bonds and terminal Tz motifs onto SiNWS was carried out via a 3-step procedure³⁵ FIG. 6. To confirm successful preparation of Tz-grafted SiNWS, X-ray photoelectron spectroscopy (XPS) was employed to monitor functional group transformation step-by-step (not shown). The passive mixing behavior of the flow-through EVs in the chaotic microchannel were simulated via the dissipative particle dynamics (DPD) model¹⁸, offering a theoretical explanation on how the configuration of the EV Click Chip leads to the enhanced physical contact³⁶ between TCO-grafted HCC EVs and Tz-grafted SiNWS.

FIG. 6 is a schematic summary of the stepwise functional group transformation employed for the preparation of Tz-grafted SiNWS. Tz-grafted SiNWS was prepared via a three-step chemical modification procedure: (i) Silanization: After cleaning SiNWS by a piranha solution, the resultant SiNWS was treated by the vapor of (3-mercaptopropyl) trimethoxysilane to give HS-SiNWS; (ii) Incorporation of disulfide bond: HS-SiNWS was reacted with OPSS-PEG-NH2 in DMSO to introduce disulfide linkers with terminal amine groups (H2N-SiNWS); (iii) H2N-SiNWS was treated with Tz-sulfo-NHS ester in PBS solution to generate Tz-grafted SiNWS.

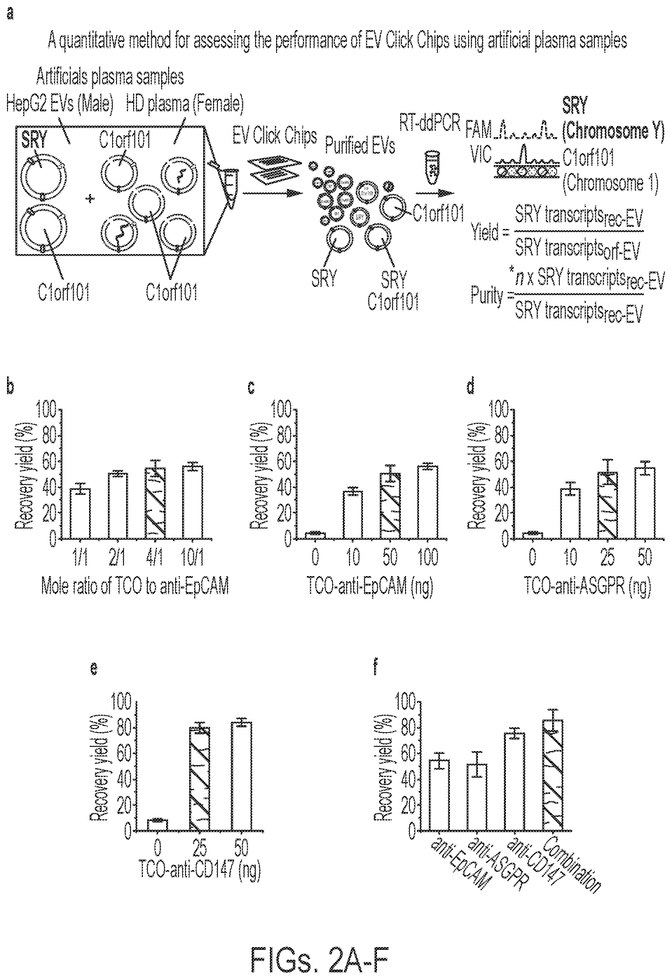

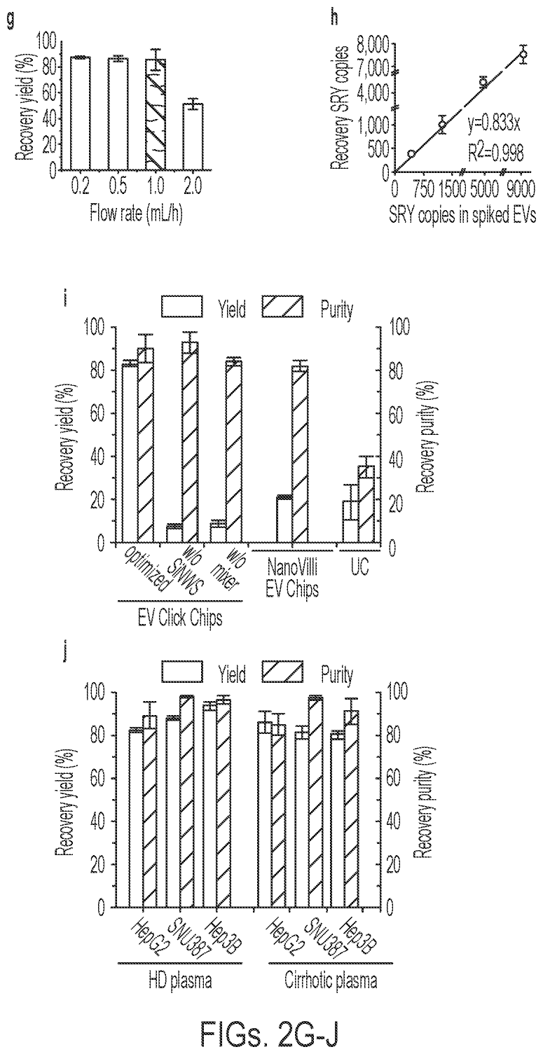

Preparation of artificial plasma samples. To allow accurate evaluation of the performance of EV Click Chip throughout the optimization process, artificial plasma samples were prepared by spiking 10-μL aliquoted HepG2 cell-derived EVs (harvested by ultracentrifugation^{37,38}) into 90-μL plasma from a female healthy donor. As shown in FIG. 2A, the presence of male HepG2 cell line-derived EVs in female plasma allows exploitation of the sex-determining region Y (SRY) gene for reliable quantification of HepG2-derived HCC EVs in purified EV samples since the SRY gene is absent in female healthy donor plasma. RT-ddPCR Assay for quantification of both SRY and C1orf101 transcripts. A RT-ddPCR assay (FIG. 2A) was used to quantify the copy numbers of the SRY and C1orf101 transcripts (encoded on Chromosome Y and Chromosome 1, respectively) in the artificial plasma samples before and after purification by EV Click Chips. The results can be used to calculate the recovery yield and purity throughout the optimization process. The copy numbers of SRY transcripts in the original 10-μL aliquoted HepG2 EVs and the EV Click Chip-recovered HepG2 EVs were denoted as SRY transcriptsori-EV and SRY transcriptsrec-EV, respectively. The EV recovery yield observed for EV Click Chip under a given condition can be obtained from the following equation:

$$Hep G 2 E V recovery yield = \frac{S R Y {transcripts}_{rec - E V}}{S R Y {transcripts}_{ori - E V}} \qquad ( 1 )$$

In order to obtain the purity of the EVs recovered by EV Click Chips, the intrinsic ratios between C1orf101 and SRY transcripts in aliquoted HepG2 EVs were measured across a wide range of concentrations. The ratios between C1orf101 and SRY transcripts exhibited a consistent linear correlation (y=1.95 x, R²=0.999). With the C1orf101-to-SRY ratio determined as 1.95, the purity of the EVs harvested from EV Click Chips is then calculated as the ratio of the recovered SRY transcripts (contributed by recovered HepG2 EVs only) to the C1orf101 transcripts (contributed by both recovered HepG2 EVs and the non-specifically captured background plasma-derived EVs, denoted as C1orf101 gene rec-EV) using the following equation:

$$H C C E V recover purity = \frac{S R Y {transcripts}_{rec - E V}}{C 1 orf 101 {transcripts}_{rec - E V}} \times {1.95}^{*} \qquad ( 2 )$$

$${*}^{} 1.95 is specific to Hep G 2 E Vs .$$

For HCC cell lines without SRY transcripts, cancer cell-derived EVs were spiked into the plasma from male donors, and the EV recovery yield and purity can be calculated using equations below:

Artificial plasma samples were prepared by spiking a) 10-μL aliquoted HepG2 cell-derived EVs into 90-μL plasma from a female healthy donor or female cirrhotic patient, b) 10-μL aliquoted SNU387 cell-derived EVs into 90-μL plasma from a male healthy donor or male cirrhotic patient and c) 10-μL aliquoted Hep 3B cell-derived EVs into 90-μL plasma from a male healthy donor or male cirrhotic patient. The EV recovery yield of the male HCC cell line (HepG2) observed for EV Click Chip can be obtained from the following equation (the copy numbers of SRY transcripts in the original 10-μL aliquoted HepG2 EVs and the EV Click Chip-recovered HepG2 EVs were denoted as SRY transcriptsori-EV and SRY transcriptsrec-EV, respectively):

$$Hep G 2 E V recovery yield = \frac{S R Y {transcripts}_{rec - E V}}{S R Y {transcripts}_{ori - E V}} \qquad ( 1 )$$

The purities of the male HCC cell line (HepG2) EVs harvested from EV Click Chips were calculated as the ratio of recovered SRY transcripts (contributed by recovered HepG2 EVs only) to C1orf101 transcripts (contributed by both recovered HepG2 EVs and the non-specifically captured background female plasma-derived EVs, denoted as C1orf101transcriptsrec-EV) using the following equation:

$$H C C E V recover purity = \frac{S R Y {transcripts}_{rec - E V}}{C 1 orf 101 {transcripts}_{rec - E V}} \times {1.95}^{*} \qquad ( 2 )$$

$${*}^{} 1.95 is specific to Hep G 2 E Vs .$$

For HCC cell lines without SRY transcripts (SNU 387, Hep3B), cancer cell-derived EVs were spiked into the plasma from a male donor or male cirrhotic patient, and the EV recovery yields and purities can be calculated using the following equations:

$$Yield = \frac{C 1 orf 101 {transcripts}_{rec - EV} - n \times S R Y {transcripts}_{rec - EV}}{C 1 orf 101 {transcripts}_{ori - EV}} \qquad ( 3 )$$

$$Purity = 1 - \frac{n \times S R Y {transcripts}_{rec - EV}}{C 1 orf 101 {transcripts}_{rev - EV}} \qquad ( 4 )$$

FIGS. 2A-2J are data graphs showing results of optimization of EV Click Chips using artificial plasma samples. In FIG. 2A, a quantitative method was developed for evaluating the performance of EV Click Chips using artificial plasma samples, prepared by spiking HepG2 EVs into plasma from a female healthy donor. A RT-ddPCR assay was employed to quantify the copy numbers of the SRY and C1orf101 transcripts in the purified EV samples to calculate the recovery yield and purity. FIG. 2B shows the recovery rates observed for EV Click Chips at different TCO-to-anti-EpCAM mole ratios (n=3). FIGS. 2C-2F show the recovery rates obtained in the presence of individual and combined antibody capture agents, i.e., (FIG. 2C) anti-EpCAM (n=3), (FIG. 2D) anti-ASGPR (n=3), (FIG. 2E) anti-CD147 (n=3), and (FIG. 2F) combination of the aforementioned 3 capture agents (n=3). FIG. 2G shows the recovery rates as the function of different flow rates. FIG. 2H shows dynamic ranges of EV recovery rates observed for EV Click Chips using artificial sample containing 0 to 9000 copies of SRY transcripts. FIG. 2I shows HepG2 EV recovery performance observed for i) EV Click Chips, the devices without embedded silicon nanowires in SiNWS or herringbone features in the PDMS chaotic mixer, ii) the devices based on immunoaffinity EV capture (NanoVilli Chips) using the antibody cocktail concentration optimized for EV Click Chips, and iii) ultracentrifugation approach. FIG. 2J shows general applicability of EV Click Chips for HCC EV recovery performance was validated using six artificial samples prepared by spiking three different HCC EVs (collected from HCC cell lines, i.e., HepG2, SNU387, and Hep3B) into two types of plasma samples (collected from either healthy donors or liver cirrhotic patients).

HCC EV purification with EV Click Chips. Prior to conducting HCC EV purification (capture/release) studies, TCO motif was covalently conjugated onto each antibody agent (FIG. 1A), and the TCO-conjugated antibody agents were incubated with the artificial or clinical plasma samples for 30 min at room temperature. In each study (FIG. 2A), a 100-μL artificial plasma sample was introduced into an EV Click Chip, in which the click chemistry, i.e. inverse-electron-demand Diels-Alder cycloaddition between Tz and TCO with a rate constant³⁹ of 10⁴ M_{-1·s}⁻¹, mediated the rapid and irreversible immobilization of HCC EVs on SiNWS. Subsequently, 100 μL DTT (50 mM) was introduced into the EV Click Chips to achieve disulfide cleavage-driven EV release.

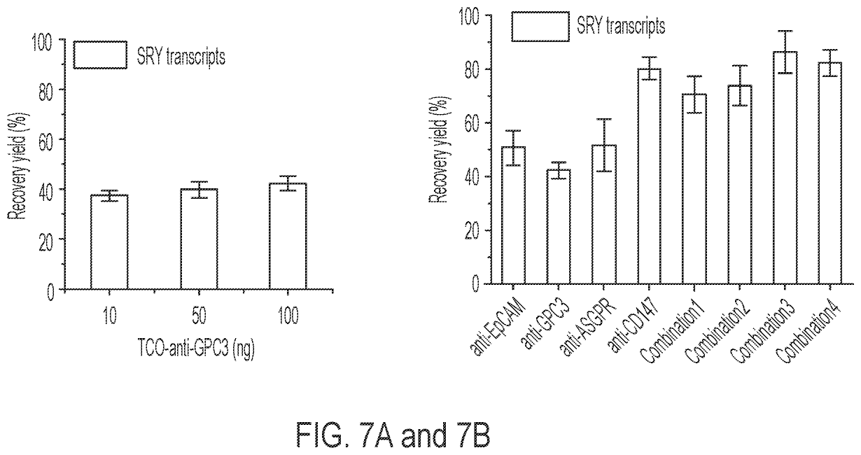

Surface marker selection and a multi-marker cocktail optimization for HCC EV capturing. Using data in the published literature^{40,41,25} that identified surface markers highly expressed in HCC EVs, HCC-CTCs, HCC cell lines, and primary tumor tissues of HCC patients, but virtually absent in white blood cells, 4 candidate antibodies, i.e. anti-EpCAM, anti-ASGPR, anti-CD147, and anti-GPC-3, directed against the corresponding surface markers were selected in order to achieve desired sensitivity and specificity of recognizing and capturing HCC EVs. The aforementioned RT-ddPCR assay was employed to assess the EV recover yield of EV Click Chips using artificial plasma samples in the presence of the individual antibodies and their cocktail mixtures. All experiments were performed at the optimum flow rate of 1 mL h₋₁ according to earlier experiences on developing NanoVilli EV Chip¹⁸. The number of TCO motif grafted on an antibody capture agent could affect the purification performance of EV Click Chips, thus first examined was how the TCO-to-anti-EpCAM mole ratios correlate with the EV recovery yields at 50-ng anti-EpCAM. FIG. 2B summarizes the recovery yields observed for EV Click Chip at different TCO-to-anti-EpCAM mole ratios, an optimal recovery yield was achieved at the TCO-to-anti-EpCAM ratio of 4:1. Under this TCO-to-antibody ratio, the concentrations of individual candidate antibodies was optimized, suggesting that the optimal amounts of anti-EpCAM (FIG. 2C), anti-ASGPR (FIG. 2D), anti-CD147 (FIG. 2E), and anti-GPC-3 (FIG. 7A) is 50, 25, 25, and 50 ng, respectively. Using the optimal concentrations for the individual antibodies, the HCC EV recovery rates of different antibody cocktails was compared, and the data summarized in FIG. 2F and FIG. 7B showed that the combination of anti-EpCAM, anti-CD147, and anti-ASGPR outperformed any single antibodies or other antibody combinations.

FIGS. 7A and 7B are data graphs showing results for antibody and antibody cocktail optimization and selection. FIG. 7A shows optimization of the anti-GPC3 concentration for recovering HepG2-derived EVs. FIG. 7B shows comparison of single antibodies and antibody cocktails for recovering HepG2-derived EVs.

Optimization of Click Chips for HCC EV purification. To further optimize the device performance, how the flow rates affect the recovery yield of HepG2 EVs was studied. 100-μL artificial plasma samples pre-incubated with the optimal antibody cocktail were introduced into EV Click Chips at flow rates ranging from 0.2 to 2.0 mL h⁻¹, and >85% average recovery yields were observed at the flow rates of 0.2 to 1.0 mL h⁻¹ (FIG. 2G). Considering the potential RNA degradation during EV purification, a faster flow rate of 1.0 mL h⁻¹ was chosen. The dynamic range of EV Click Chips using artificial plasma samples containing 0 to 9000 copies of SRY transcripts per 100-μL volume was used. EV Click Chips exhibited consistent recovery yields (y=0.833x, R²=0.998) that are sufficient to purify HCC EV in clinical plasma samples (FIG. 2H). To understand the crucial roles of the embedded silicon nanowires in SiNWS, the herringbone features in a PDMS chaotic mixer, and click chemistry-mediated EV capture, controlled experiments were carried out (not shown) using i) the devices without embedded silicon nanowires in SiNWS or herringbone features in the PDMS chaotic mixer, and ii) the devices based on immunoaffinity EV capture¹⁸ (Nano Villi Chips), in parallel with EV Click Chips and the ultracentrifugation approach³⁸. EV Click Chips exhibited a recovery yield of 83.3±1.4% and purity of 90.2±6.2%, which were significantly higher than those observed for the controls (FIG. 2I). To test the general applicability of EV Click Chips and the optimized EV purification condition, the performance of EV Click Chips was further validated using six artificial samples prepared by spiking three different HCC EVs (collected from HCC cell lines, i.e., HepG2, SNU387, and Hep3B) into two types of plasma samples (collected from either healthy donors or liver cirrhotic patients). Overall, EV Click Chips achieved recovery yields ranging from 81.2% to 94.6% and purity between 85.9% and 99.1% (FIG. 2J).

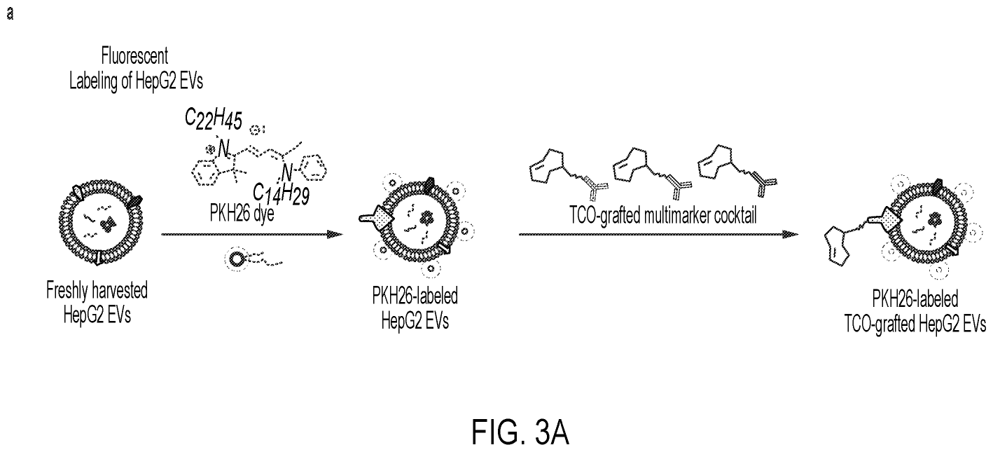

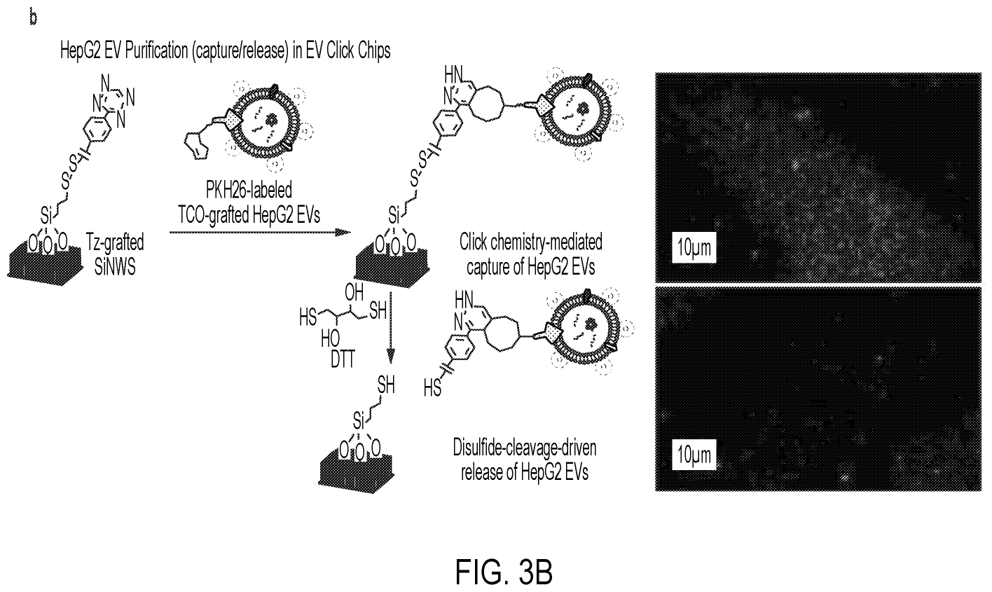

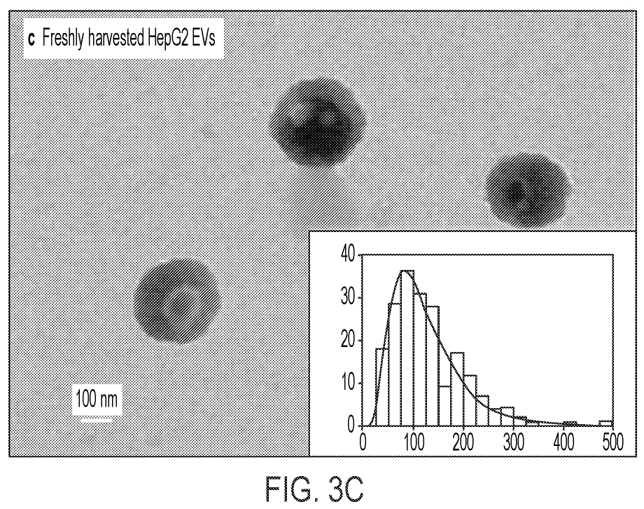

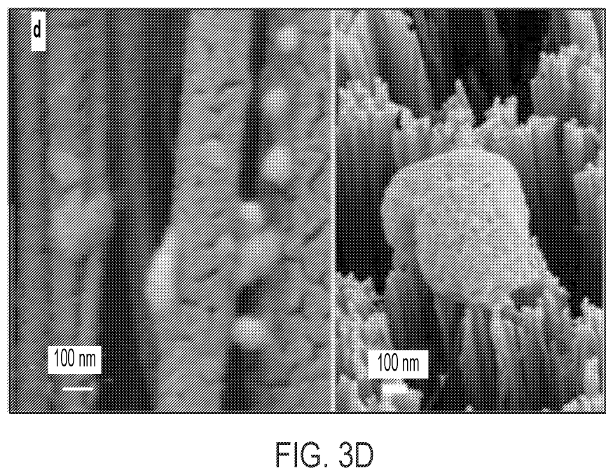

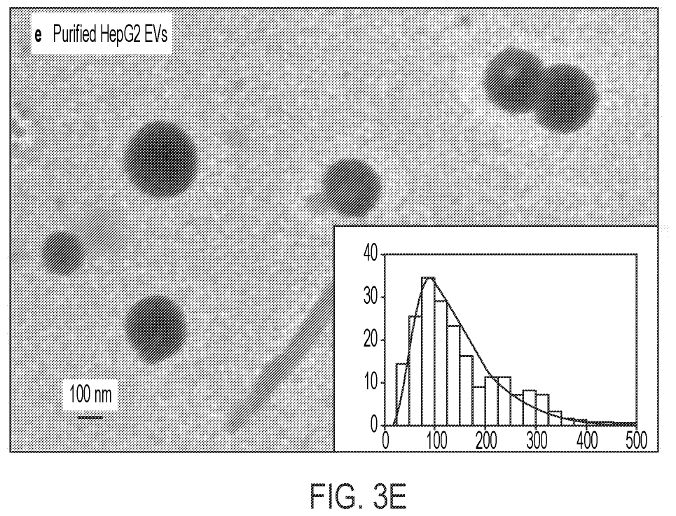

Characterization of HCC EVs purified by EV Click Chips. To better understand the working mechanisms associated with the click chemistry-mediated EV capture and disulfide cleavage-driven EV release, fluorescence microscopy, transmission electron microscopy (TEM), dynamic light scattering (DLS), and/or scanning electron microscopy (SEM) were employed to characterize the EV sizes and EV/SiNWS interfaces during the EV purification process, in which freshly harvested HepG2 EVs in PBS were used as a model system. To allow direct tracking of the capture and release processes of HCC EVs in EV Click Chips, HepG2 EVs were first labeled (FIG. 3A) with PKH26 dye (Sigma-Aldrich), which stains EV membranes by intercalating their aliphatic portion into the exposed lipid bilayer of the EVs. After click chemistry-mediated capture of PKH26-labeled TCO-grafted HepG2 EVs (FIG. 3B), the SiNWS were subjected to fluorescence microscopy (Nikon, 90i) imaging. The micrographs unveiled that PKH26-labeled TCO-grafted HepG2 EVs were trapped on the surfaces of Tz-grafted SiNWS. Upon exposure to DTT, the surface linkers anchoring the HepG2 EVs onto the SiNWS were cleaved, leading to the release of PKH26-labeled HepG2 EVs. Fluorescent microscopic imaging revealed dramatic reduction of fluorescent signals on the SiNWS. FIG. 3C shows a representative TEM image of freshly harvested HepG2 EVs after uranyl acetate negative staining. These HepG2 EVs exhibited cup- or spherical-shaped morphologies with sizes ranging between 40 and 500 nm in diameter measured by TEM (insert of FIG. 3C), consistent with those observed by DLS. FIG. 3D shows a cross-sectional SEM image of Si nanowires with HepG2 EVs captured onto both the sidewalls (left) and the tops of the nanowires (right). TEM images (FIG. 3E) of these purified HepG2 EVs suggested that the purified HepG2 EVs retained intact morphologies with a similar size distribution (insert) to the freshly harvested HepG2 EVs.

FIGS. 3A-3E are illustrations and microscopic images showing the characterization of HepG2 EVs purified by EV Click Chips. FIG. 3A shows fluorescent labeling of HepG2 EVs by PKH26 dye, followed by incubation with TCO-grafted antibody cocktail, giving PKH26-labeled TCO-grafted HepG2 EVs. FIG. 3B shows tracking the purification (capture/release) process of HepG2 EVs in EV Click Chips using fluorescent microscopy. After click chemistry-mediated capture, PKH26-labeled HepG2 EVs were immobilized on SiNWS, as confirmed by the fluorescence micrograph (upper). Upon exposure to DTT, the surface linkers that anchored the PKH26-labeled HepG2 EVs onto SiNWS were cleaved, leading to the release of PKH26-labeled HepG2 EVs, as confirmed by fluorescence micrograph (Lower). FIG. 3C shows representative TEM images of HepG2 EVs in bulk solution before capture. Inset: Size distribution (n=338 diameters=30-500 nm) of HepG2 EVs, measured by TEM. FIG. 3D shows SEM images of HepG2 EVs (colored in pink) on the sidewall (left) and tops (right) of the SiNWS. FIG. 3E shows representative TEM images of HepG2 EVs after being released from the chip. Inset: Size distribution (n=363, diameters=40-500 nm) of HepG2 EVs, measured by TEM. Scale bar, 100 nm.

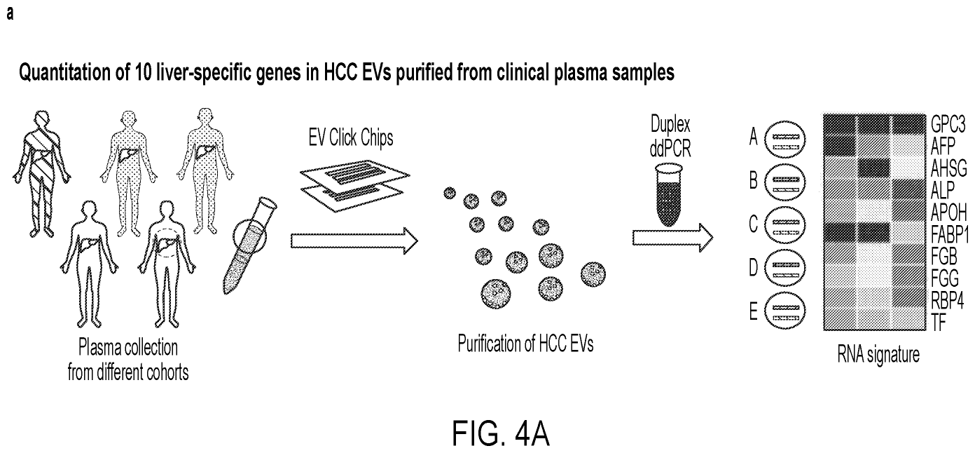

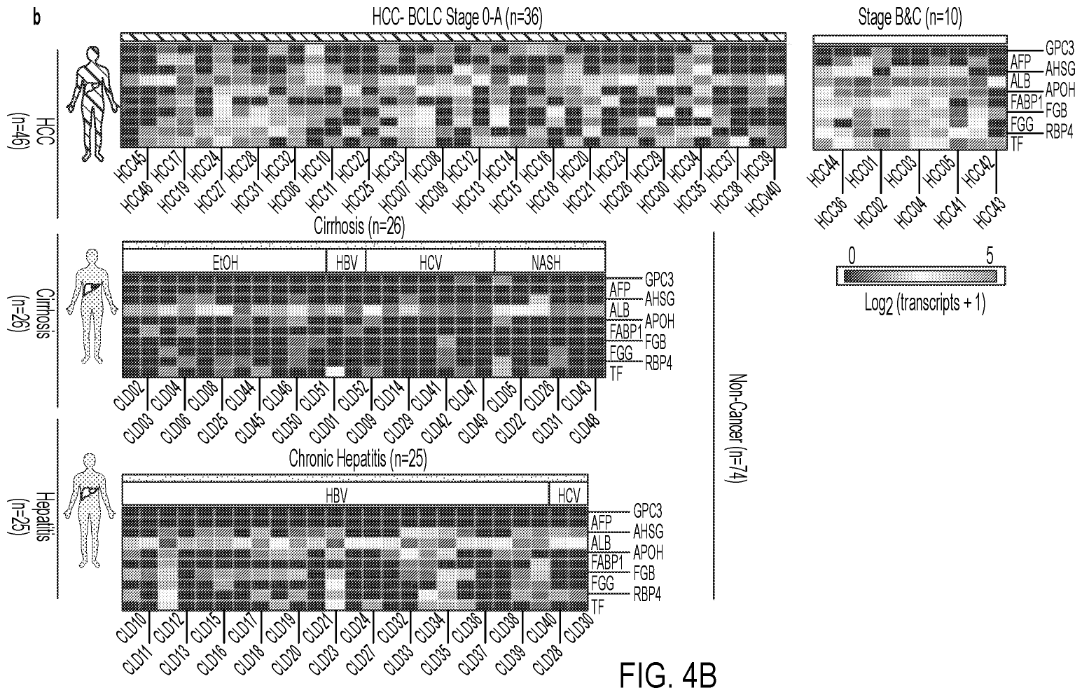

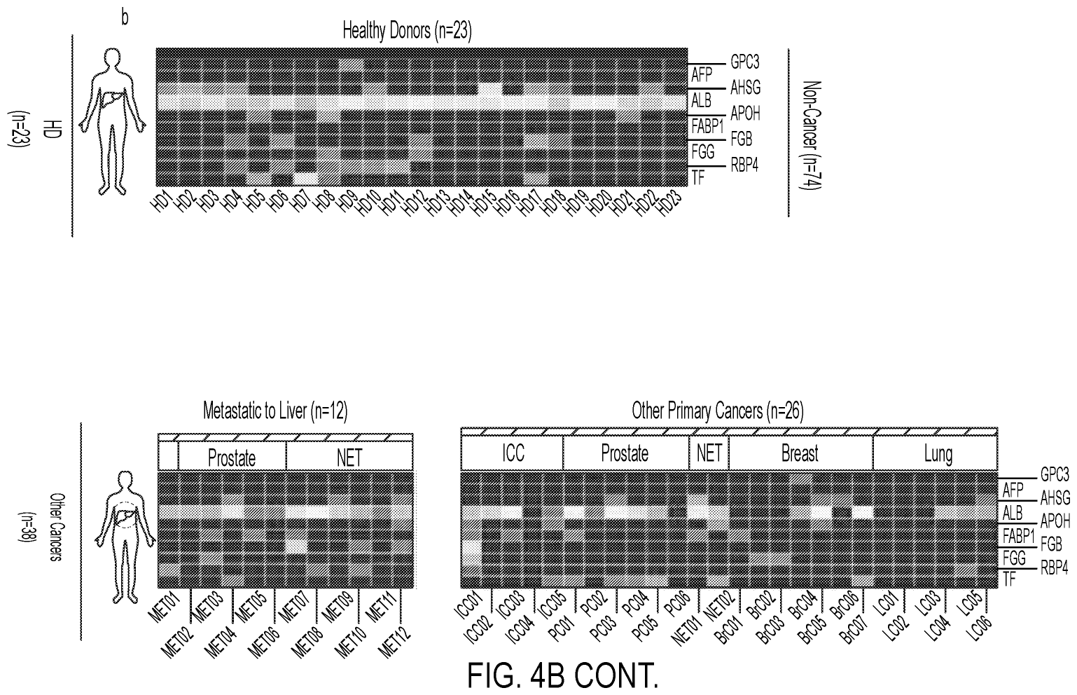

Quantification of 10 HCC-specific genes using HCC EVs purified from clinical samples. Under the optimal HCC EV purification condition, a workflow was achieved (FIG. 4A) by which EV Click Chips were employed to purify HCC EVs in 158 plasma samples (0.5 mL) from five cohorts, including (i) healthy donors (n=23, median age=49 y); (ii) patients with chronic hepatitis (n=25, median age=57 y); (iii) patients with liver cirrhosis with etiologies of hepatitis B virus (HBV), hepatitis C virus (HCV), alcoholic liver disease, or Non-alcoholic Steatohepatitis (NASH), who were being routinely monitored for HCC development (n=26, median age=61 y); (iv) newly diagnosed, treatment-naive patients with HCC (n=46, median age=66 y); and (v) patients with primary malignancies other than HCC, with or without liver metastases (n=38, median age=58 y). Subsequently, the purified HCC EV samples were then subjected to RT-ddPCR quantification of the 10 well-validated HCC-specific genes, including alpha-fetoprotein (AFP), glypican 3 (GPC3), albumin (ALB), apolipoprotein H (APOH), fatty acid binding protein 1 (FABP1), fibrinogen beta chain (FGB), fibrinogen gamma chain (FGG), alpha 2-HS glycoprotein (AHSG), retinol binding protein 4 (RBP4), and transferrin (TF)³³. Clinical annotation of all the plasma samples was performed by a clinician blinded to the assay. The HCC EV-derived 10-gene digital readouts obtained for the individual participants were summarized in the heat maps (FIG. 4B). As depicted in the heat maps, HCC patients were grouped according to BCLC (Barcelona Clinic Liver Cancer) staging system⁴² (FIG. 4B, upper). Higher signals were observed in HCC patients, compared with the Non-Cancer groups (liver cirrhosis, chronic hepatitis, and healthy donors) (FIG. 4B, Middle), or patients with cancers other than HCC, including intrahepatic cholangiocarcinoma (ICC), breast cancer, lung cancer, prostate cancer, midgut neuroendocrine tumor (NET), and cancers of nonhepatic origin metastatic to the liver (MET) (FIG. 4b, Lower). Moreover, signal differences among HCC patients with different stages defined by BCLC⁴², Milan criteria⁴³, or United Network for Organ Sharing down-staging (UNOS DS)⁴⁴ Criteria, with advanced-stage HCC patients exhibiting higher signals from multiple transcripts of interest than early-stage HCC patients. Overall, HCC samples exhibited positive signals for 4 to 9 transcripts, reflecting successful detection of the 10-gene panel in HCC EVs purified from 0.5-mL plasma samples.

FIGS. 4A and 4B show results for RT-ddPCR assay for quantification of 10 HCC-specific mRNA transcripts in HCC EVs purified by EV Click Chips. FIG. 4A shows a general workflow developed for conducting HCC EV purification, followed by quantification of 10 HCC-specific mRNA transcripts in the purified HCC EVs. FIG. 4B shows heat maps depicting relative signal intensities for each gene expression of the 10 HCC-specific genes across different patient cohorts. (Upper) Patients with newly diagnosed HCC are grouped according to BCLC staging system from early stages to advanced stages. (Middle) Patients with Non-Cancer, including liver cirrhosis (n=26), chronic hepatitis (n=25) and healthy donors (n=23). (Lower) Patients with cancers other than HCC (n=38): cancers of nonhepatic origin metastatic to the liver (MET, n=12); other primary cancers (n=26), including intrahepatic cholangiocarcinoma (ICC), prostate cancer, midgut neuroendocrine tumor (NET), breast cancer, and lung cancer. Primary copy numbers are log-2-transformed and scaled to the highest value for each gene.

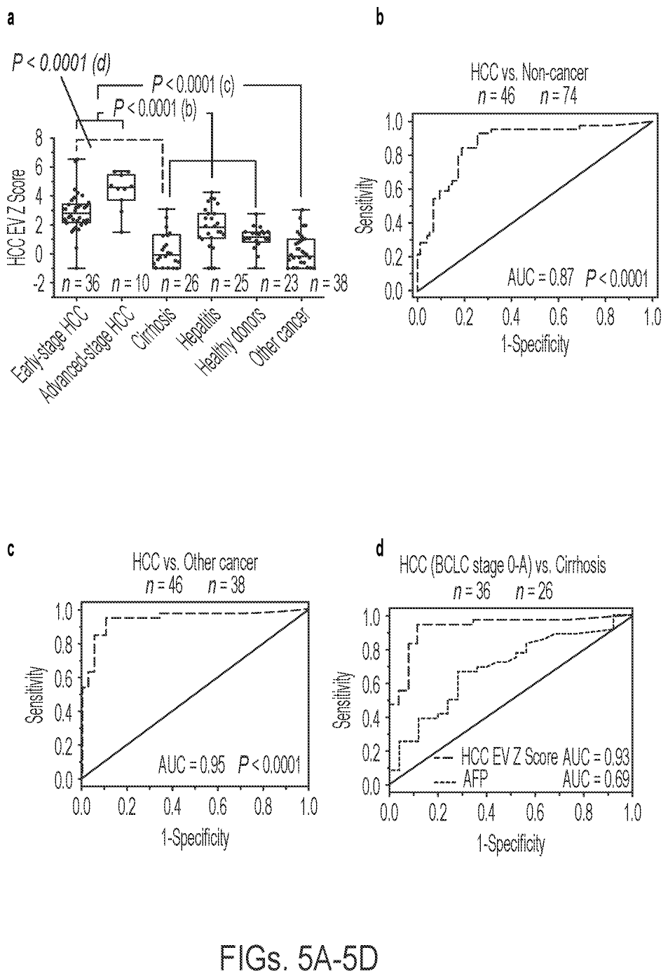

HCC EV Z Scores for HCC detection. HCC EV Z Scores for each sample were computed based on the expression of 10 genes in purified HCC EVs using the weighted Z-score method⁴⁵. The copy numbers of the 10 genes were combined into the single HCC EV Z Scores. As depicted in the box plot (FIG. 5A), the HCC EV Z Score of the HCC cohort is significantly higher (***P<0.001) than any other cohort, (i.e. Liver Cirrhosis, Chronic Hepatitis, Healthy Donor, and Other Cancer).

FIGS. 5A-5D are data graphs showing statistical analysis on HCC EVZ Scores in different cohorts. FIG. 5A are box plots representing the HCC EV Z Scores for different patient cohorts including early-stages HCC (n=36), advanced-stage HCC (n=10), cirrhosis (n=26), hepatitis (n=25), healthy donors (n=23) and other cancers (n=38). Whiskers ranging from minima to maxima, median and 25-75% IQR shown by box plots. Significant differences between different groups were evaluated using one-way ANOVA. FIGS. 5B and 5C are ROC curves for HCC EV Z Scores in (FIG. 5B) HCC versus non-cancer (i.e. cirrhosis, hepatitis, and healthy donors) (AUC=0.87, P=9.64E-12, 95% CI, 0.80 to 0.94), (FIG. 5C) HCC versus other cancer (AUC=0.95, P=1.79E-12, 95% CI, 0.90 to 1.00). FIG. 5D are ROC curves comparing HCC EV Z Scores (AUC=0.93, P=1.02E-8, 95% CI, 0.86 to 1.00) with the serum biomarker alpha-fetoprotein (AFP) level (AUC=0.69, P=0.013, 95% CI, 0.55 to 0.83) for differentiating early-stage HCC (BCLC, stage 0-A) vs. at-risk cirrhosis. Barcelona Clinic Liver Cancer (BCLC); ROC, receiver operator characteristic.

HCC largely occurs in pre-existing chronic liver diseases⁴⁶, but can also develop without such pre-conditions. Thus, subgroup analysis was performed to test the feasibility of distinguishing HCC patients from the Non-cancer group (Liver Cirrhosis, Chronic Hepatitis and healthy donors) using ROC curve. The AUC for the HCC EV Z Score for distinguishing HCC from Non-cancer was 0.86 (95% CI, 0.79 to 0.93; sensitivity=85.2%, specificity=80.1%, FIG. 5B). It's also crucial to assure that the HCC EV assay is specific for HCC, and can discriminate HCC from other primary (cholangiocarcinoma) and secondary liver malignancies (metastases to liver from extrahepatic primary tumors). The subgroup analysis was also performed to test the feasibility of distinguishing HCC patients from other cancers. The AUC for distinguishing HCC from other cancers was 0.95 (95% CI, 0.90 to 1.00; sensitivity=95.7%, specificity=89.5%, FIG. 5C).

HCC EV Z Scores for early HCC detection. HCC occurs in the background of liver cirrhosis in over 80% of cases⁴⁶, emphasizing the need to develop an early detection method to identify localized HCC from at-risk liver cirrhosis populations, in turn providing great hope for curative therapy. Among all patients with HCC, the HCC EV Z Scores were highly differentiated between early-stage HCC and advanced-stage HCC according to BCLC staging⁴² (P<0.01), Milan criteria⁴³ (P<0.01), and UNOS DS Criteria⁴⁴ (P<0.01). These results indicate promise that the HCC EV Z Score generated from HCC EVs purified by EV Click Chips may serve as a noninvasive predictor for early detection of HCC. To explore the potential for the EV Click Chip-based HCC-EV Assay to detect early-stage HCC, the feasibility of distinguishing early-stage HCC patients (defined according to BCLC staging, Milan criteria, or UNOS DS Criteria) from at-risk liver cirrhosis patients (where HCC prevalence is higher) using ROC analysis was tested. Since serum AFP levels were available for all HCC patients and at-risk chronic liver disease patients, the performance of the HCC EV Z Score with the clinical AFP test for differentiating early-stage HCC (BCLC Stage 0-A, within Milan Criteria, or within UNOS DS Criteria) vs. at-risk liver cirrhosis patients was compared. (FIG. 5D). The HCC EV Z Score achieved better diagnostic performance with the AUC of 0.93, 0.91and 0.92 in comparison to AFP with the AUC of 0.69, 0.68 and 0.70 for distinguishing HCC patients with BCLC Stage 0-A, or within Milan Criteria, or within UNOS DS Criteria, respectively, from at-risk patients. The HCC EV Z Score outperformed the AFP regardless of what clinical staging system was used for defining early-stage HCC. This data serves as a promising proof-of-concept for utilizing HCC-EVs purified by EV Click Chips as a diagnostic tool for early detection of HCC in at-risk populations.

### Discussion

The results above demonstrate development of a new HCC EV purification system, i.e. EV Click Chips, by uniquely integrating several novel strategies including covalent chemistry-mediated EV capture/release, a multimarker antibody cocktail, nanostructured substrates, and a microfluidic chaotic mixer, promising rapid and effective purification of HCC EVs with intact mRNA cargo. By coupling EV Click Chips with a downstream RT-ddPCR assay designated to quantify 10 well-validated HCC-specific mRNA transcripts³³, the resulting HCC EV-derived 10-gene digital readouts exhibited great potential for non-invasive early detection of HCC. A unique feature of EV Click Chips is the exploration of the covalent chemistry-mediated EV purification (capture/release) process through two consecutive steps: i) click chemistry-mediated EV capture and ii) disulfide cleavage-driven EV release. Click chemistry is a class of rapid bioorthogonal organic reactions frequently used for bioconjugation, e.g., coupling of biomolecules with substrates of interest (e.g., reporter molecules). Due to the very low number of antigens present on the surface of individual EVs, immunoaffinity-based EV capture approaches, which are driven by the dynamic binding between a pair of antigen (on EV) and antibody (on the substrate), often suffer from poor EV capture performance and high background issues. This problem can be resolved by replacing immunoaffinity-mediated capture to click-chemistry-mediated capture. Among different click chemistry reactions, the inverse-electron-demand Diels-Alder cycloaddition⁴⁷ between Tz and TCO motifs (a rate constant³⁹ of 10⁴ M−1·s⁻¹) was selected given their balanced chemical properties concerning both stability and reactivity, and the lack of a need for the presence of a catalyst. The ligation between Tz-grafted SiNWS and TCO-grafted EVs is rapid, specific, irreversible, and insensitive to biomolecules, water, and oxygen, leading to specific, rapid, and irreversible immobilization of the EVs with improved capture efficiency and reduced nonspecific trapping of particles in the background. Furthermore, considering the fact that HCC EVs are secreted by highly heterogeneous HCC22-24 cells, it is conceivable that no single capture agent can achieve sufficient performance to capture HCC EVs. Therefore, it is necessary to develop an antibody cocktail to recognize and capture HCC EVs from clinical samples, allowing for sensitive and specific detection of HCC-derived EVs across all disease stages. The experimental data using both artificial and clinical plasma samples showed that significantly greater EV capture yield and purity was achieved when utilizing a 3-antibody combination cocktail compared to each single antibody alone (i.e., anti-EpCAM, anti-ASGPR, and anti-CD147). Moreover, based on previous experience in exploring the combined use of nanostructured immunoaffinity substrates and PDMS microfluidic chaotic mixers to achieve highly efficient capture of targeted particles (i.e., CTCs and EVs) in peripheral blood, integrating this device configuration with click chemistry-mediated EV capture and a multimarker antibody cocktail offers the most sensitive and specific technology for capturing HCC EVs with a minimal level of background. This approach also allows for more effective conjugation of the TCO-grafted antibody cocktail onto the majority of HCC EVs in a small volume of solution, facilitating the click-chemistry-mediated HCC EV capture onto Click Chips. Following the click chemistry-mediated capture of EV Click Chips, the subsequent disulfide cleavage-driven HCC EV release confers the second layer of specificity to the HCC EVs purification process, further improving the purity of recovered HCC EVs.

The combined use of a multimarker antibody cocktail and EV Click Chips could possibly lead to recovering EVs which are not of HCC origin. For example, anti-EpCAM could capture EVs from other epithelial tissues. To address this concern, the RT-ddPCR assay capable of quantifying 10 HCC-specific genes as a downstream readout for the purified HCC EVs was adopted. These 10 HCC-specific genes were selected from tissue lineage-associated transcripts expressed in liver cells but absent in the EVs released from blood cells and other tissues. The resulting 10-gene digital readouts were predominantly contributed by HCC EVs, thus conferring the third layer of specificity for detecting HCC EVs.

In the process of optimizing the EV Click Chip, a novel quantitative evaluation method was developed that has addressed the dire need of assessing the purification performance (EV recovery yield and purity) of the EV Click Chip. Due to the lack of highly prevalent mutations in HCC, a novel system was devised where the SRY gene encoded on Chromosome Y from a male HCC cell line would be utilized as an artificial HCC biomarker. An artificial plasma sample was first prepared by spiking EVs from a male HCC cell line into plasma from a female healthy donor, and utilized quantification of the SRY transcript as a means for distinguishing and quantifying spiked HCC EVs. RT-ddPCR assay was then adopted for counting the copy numbers of the SRY and C1orf101 transcripts (in Chromosome Y and Chromosome 1, respectively) in the purified HCC EVs. This method is more convenient and quantitative than the existing methods¹⁷ that required pre-labeling or pre-transfection of EVs with specific transcripts messages. Moreover, this method is broadly applicable to the optimization of any other tumor-derived EV purification platforms before clinical study.

Current diagnostics for HCC fall into two main categories: radiologic imaging and blood-based biomarker tests⁴⁸. However, the diagnostic performance of these modalities (i.e., ultrasonography and serum AFP) is inadequate, particularly for the diagnosis of early-stage HCC⁴⁹. When liquid biopsies emerged, they were hailed as a possible screening tool for cancer, but proved to lack sufficient specificity and sensitivity for early detection of cancer^{50,51}, The EV Click Chip-based HCC EV Assay for HCC diagnosis was applied, where the early detection strategies are currently unsatisfactory. There have been promises on the horizon for emerging liquid biopsy-based HCC diagnostics such as ctDNA-based methylation for HCC detection⁵² and CTC-based RNA signature for HCC detection³³. Although ctDNA methylation profile using whole genome bisulfite sequencing can detect early-stage HCC⁵², its use in HCC screening may be challenging because of the relatively high cost and long turnaround time. Further, an inherent limitation of ctDNA-based methylation is its fragmentation, and it is released predominantly by cell death into the bloodstream, amid the background of DNA released from normal cells53. Moreover, although CTCs enable high specificity detection of HCC-specific mRNA signatures, the sensitivity of the CTC-based 10-gene assay for early detection of HCC is limited due to the fact that fewer CTCs are present in earlier stages of cancer⁵⁴. The 10-gene panel originally developed by the MGH group³³ for HCC CTC detection was adopted for the EV Click Chip, which takes advantage of HCC EVs. These small membrane-bound particles encapsulate HCC-specific mRNA which can be selectively isolated from total EVs even at an early stage in satisfactory quantities. The analysis of the isolated pooled HCC EVs has allowed mRNA-based detection of HCC-specific gene signatures, paving the way for early detection of HCC.

In this example, the potential clinical utilities of EV Click Chips in HCC for (i) differentiating HCC from Non-HCC, (ii) differentiating HCC from other cancers with or without metastasis to liver, and (iii) distinguishing early-stage HCC from at-risk liver cirrhosis is demonstrated. EV Click Chips exhibit dramatically improved recovery yield and purity of HCC EVs compared to commonly used EV isolation methods (i.e. ultracentrifugation). Beyond the early diagnosis of HCC from at-risk CLD patients, the resulting HCC EV digital score generated by the assay also showed the potential for HCC staging consistent with BCLC and Milan criteria and significantly augment the ability of current diagnosis and staging criteria to realize early detection of HCC and longitudinal monitoring of disease progression. The platform for HCC early diagnosis is broadly applicable to other cancer types. Since tumor-derived EVs can be efficiently isolated by targeting multiple surface markers and can carry tumor-specific genes that are absent in normal blood components, they hold considerable promise for the early detection of cancer.

### Methods

### Fabrication of Tz-grafted SiNWS

10-15 μm Si nanowires (diameter=100-200 nm) were introduced onto Tz-grafted SiNWS via a fabrication process combining photolithographic patterning and silver (Ag) nanoparticle-templated wet etching⁵⁵, offering approximately 30 times more surface areas (in contrast to a flat substrate) for facilitating click chemistry-mediated EV capture. According to the protocols published in previous study³⁴ SiNWS were fabricated by combining the photolithographic patterning and Ag nanoparticle-templated wet etching⁵⁵. In short, a p-type Si (100) wafer (Silicon Quest Int'l) was spin-coated with a thin film photoresist (AZ 5214, AZ Electronic Materials USA Corp.) using a resistivity of 10-20 Ω·cm. The Si wafer was then immersed into the etching solution containing HF (4.6 M, Sigma-Aldrich), AgNO₃ (0.2 M, Sigma-Aldrich) and deionized (DI) water after being exposed to ultraviolet light. Finally, the Ag nanoparticle-templates were removed by immersing these Si wafer into boiling aqua regia (HCl/HNO₃, 3:1 (v/v), Sigma-Aldrich) for 15 min. The SiNWS were then treated with acetone (≥99.5%, Sigma-Aldrich), followed by ethanol anhydrous (Sigma-Aldrich) wash. A disulfide linker was used to couple the Tz motifs grafted on the chips by designing a three-step chemical modification procedure: (i) Silanization: The SiNWS were first immersed in a freshly prepared piranha solution (H₂SO₄/H₂O₂, 2:1 (v/v), Sigma-Aldrich) for 1 hour, followed by rinsing with DI water and ethanol successively for three times. After drying under nitrogen flow, the resultant SiNWS were sealed in a vacuum desiccator for treatment (3-mercaptopropyl) trimethoxysilane vapor (211.4 mg, 200 μL, Sigma-Aldrich) for 45 min to introduce thiol groups onto the SiNWS. (ii) Incorporation of disulfide bond: OPSS-PEG-NH₂ (0.30 mg, 3.8 mM, Nanocs Inc.) was incubated with freshly prepared HS-SiNWS in dimethyl sulfoxide (DMSO, 200 μL) solution for 2 hours to introduce disulfide linkers with terminal amine groups. Then the amine-terminated SiNWS (H₂N-SiNWS) were rinsed with ethanol three times. (iii) To graft Tz motifs, the H₂N-SiNWS was incubated with Tz-sulfo-NHS ester (0.32 mg. 3.8 mM, Click Chemistry Tools Bioconjugate Technology Company) in PBS (200 μL. PH=8.5) for 1 h. The resulting Tz-grafted SiNWS were rinsed with DI water three times. After drying under nitrogen flow, the Tz-grafted SiNWS were stored at −20° C. To confirm successful preparation of Tz-grafted SiNWS, X-ray photoelectron spectroscopy (XPS) was employed to monitor functional group transformation step-by-step.

### Preparation of TCO-antibody Conjugates

Goat anti human EpCAM (R&D Systems, Inc.), goat anti human CD147 (R&D Systems, Inc.), rabbit anti human ASGPR (LifeSpan BioSciences, Inc.), and sheep anti human GPC3 (R&D Systems, Inc.) were incubated with TCO-PEG+-NHS ester (0.5 mM, Click Chemistry Tools Bioconjugate Technology Company) in PBS according to different mole ratios at room temperature for 30 min respectively. The individual TCO-antibody conjugates were prepared freshly before their use.

### Cell Line Culture

HepG2, Hep 3B cell line were purchased from American Type Culture Collection and cultured in Eagle's Minimum Essential Medium with 10% fetal bovine serum (FBS), 1% GlutaMAX-I and 100 U mL⁻¹ penicillin-streptomycin (Thermo Fisher Scientific) in a humidified incubator with 5% CO₂. SNU 387 cell line was purchased from American Type Culture Collection and cultured in RPMI-1640 Medium with 10% FBS, 1% GlutaMAX-I and 100 U mL−1 penicillin-streptomycin in a humidified incubator with 5% CO2.

### Artificial Plasma Sample Preparation

HepG2, Hep 3B, SNU 387 cells were cultured in 18 Nunc EasYDish dishes (145 cm². Thermo Fisher Scientific) for 72 hours. Then the culture medium was switched to serum-free culture medium (Thermo Fisher Scientific) to starve the cells for 24-48 hours. The serum-free culture medium incubated with cells was finally collected for EV isolation. After first centrifugation at 300 g (4° C. for 10 min to remove cells and cell debris, the supernatant was collected and transferred to new tubes and centrifuged at 2800 g (4° C.) for 10 min to further eliminate the remaining cellular debris and large particles. The supernatant was carefully transferred to Ultra-Clear Tubes (38.5 mL, Beckman Coulter, Inc., USA), followed by ultracentrifugation using an Optima L-100 XP Ultracentrifuge (Beckman Coulter, Inc, USA) at 100 000 g (4° C.) for 70 min. The EV pellets at the bottom of the tubes were carefully collected and resuspended in 200 μL fresh PBS. For the artificial plasma samples, each 10 μL aliquot of EV pellets was spiked into 90 μL healthy donor's plasma or cirrhotic patients' plasma.

### Characterization of HepG2 EVs

For SEM characterization of HepG2 EVs, 10 μL pure HepG2 EVs in 100 μL PBS were run through the chips. The SiNWS were then cut to expose the cross sections of the silicon nanowire arrays. The severed SiNWS with captured HepG2 EVs were fixed in 4% PFA for 1 hour, followed by sequential dehydration through 30, 50, 75, 85, 95, and 100% ethanol solutions for 10 min each. After overnight lyophilization, the samples were sputter-coated with gold at room temperature. The images were visualized and taken under a ZEISS Supra 40VP SEM at an accelerating voltage of 10 520 keV.

For TEM characterization of HepG2 EVs, 10 μL freshly harvested HepG2 EVs or purified HepG2 EVs were deposited on the 200-mesh formvar-carbon coated EM grids for 20 min, and then the grids were transferred (membrane side down) to a 100-μl drop of 4% PFA for 10 min. After 3 times water-drop washing, the grids were treated with 2% uranyl acetate for 5 min and excess fluid was blotted by filter paper. The grids air dried before TEM imaging by JEM1200-EX (JEOL USA Inc.) at 80 kV.

For the fluorescent labeling of captured EVs, 10 μL pure HepG2 EVs in 100 μL PBS were run through the chips. 1.2 μL PKH26 dye was added into 200 μL Diluent C and mixed continuously for 30 seconds by gentle pipetting. The severed SiNWS with captured HepG2 EVs were incubated with this PKH26 dye solution at room temperature for 10 minutes. The EV Click Chips after HCC EV capture and release were observed by a fluorescence microscopy.

### EV Click Chips for HCC EV Purification

After chip assembly and leak testing according to previously described protocols¹⁸, the artificial plasma samples (100 μL) or the clinical plasma samples (500 μL) incubated with TCO-antibodies were then injected into EV Click Chip microfluidic devices. For EV release, 100 μL DTT solution (50 mM) was injected into the EV Click Chips at 1.0 mL/h and the released EVs were collected in 1.5 mL RNase-free Eppendorf tubes for subsequent RNA extraction.

### RNA Extraction and RT-ddPCR

The HCC EVs recovered from EV Click Chips were lysed by 700 μL QIAzol Lysis Reagent. RNA was extracted using a miRNeasy Micro Kit (Qiagen, German) according to the manufacturer's instructions. Then the complementary DNA (cDNA) was synthesized using a Thermo Scientific Maxima H Minus Reverse Transcriptase Kit according to the manufacturer's instructions. For the optimization experiments, cDNA was subjected to detect SRY transcripts and C1orf101 transcripts using duplex ddPCR in one tube with two fluorescence filters (i.e. FAM and VIC). For the clinical samples, 10 μL of total cDNA (12 μL) was divided into 5 tubes to detect the 10 genes with two fluorescence filters in each tube. ddPCR experiments were performed on a QX 200 system (Bio-Rad Laboratories, Inc.) according to the manufacturer's instructions. Data were analyzed using the QuantaSoft™ software to quantify the corresponding copy numbers of gene transcripts detected in each assay.

### Enrollment of HCC Patients and Control Cohorts

All the participants in this study were enrolled between October 2016-October 2019. Treatment—naïve HCC patients across all stages were enrolled in this study. HCC patients who had other malignant tumors, or severe mental diseases were excluded. The control cohorts consisted of patients with chronic liver disease, other cancers with or without metastasis to liver, and healthy donors. A detailed description of each control cohort and clinical characteristics can be found in the supporting information. All patients and healthy donors provided written informed consent for this study according to the IRB protocol (#14-000197) at UCLA and (STUDY00000066) at Cedars-Sinai Medical Center.

### Clinical Blood Sample Processing

Peripheral venous blood samples were collected from fasting patients or healthy donors with the written informed consent from each patient or healthy donor according to the institutional review board (IRB) protocols at UCLA and Cedars-Sinai Medical Center. Each 8.0 mL blood sample was collected in a BD Vacutainer glass tube (BD Medical, Fisher Cat. #02-684-26) with acid citrate dextrose. Samples were processed according to the manufacturer's protocol within 4 h of collection.

The final plasma samples were collected for the HCC EV study after centrifugation at 10,000 g for 10 min. 500 μL plasma samples were then incubated with TCO conjugated anti-EpCAM (250 ng), anti-ASGPR (125 ng) and anti-CD147 (125 ng) at room temperature for 30 min before being loaded into the EV Click Chips for the HCC EV purification.

### Statistical Analysis

The EV recovery yields and purities are expressed as Mean±S.E.M. Significant differences between different groups were evaluated using one-way ANOVA. The 10-gene HCC EV Z Score, which represents the likelihood estimate of 10-gene activation, was computed from the RNA expression of the 10 genes using a weighted Z score method⁴⁵ in R studio. After mean centering of expression data across the samples, HCC EV Z Scores were computed by the error-weighted mean of the expression values of the 10 genes in a sample. ROC curve was applied to evaluate the diagnostic performance for each parameter using MedCalc software.

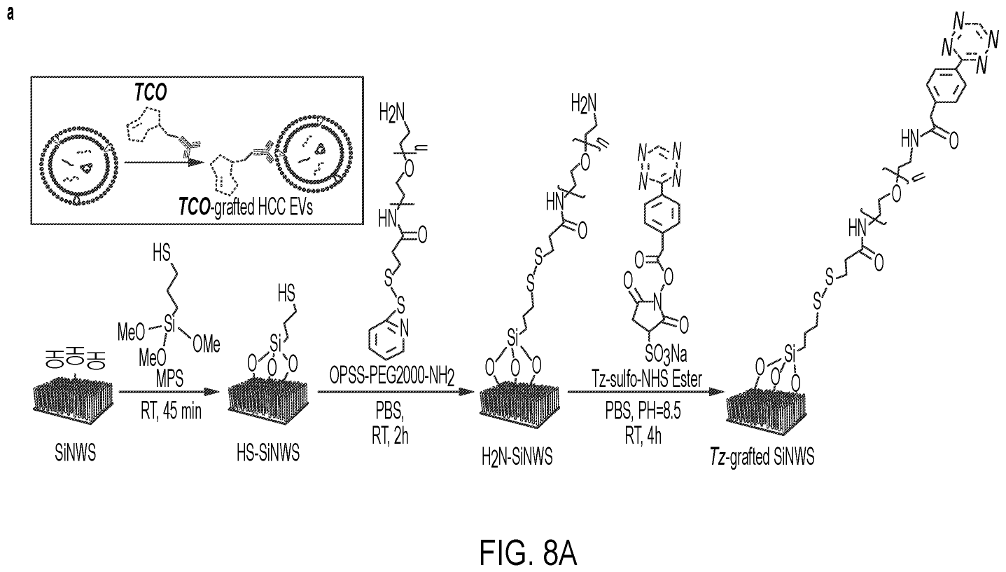

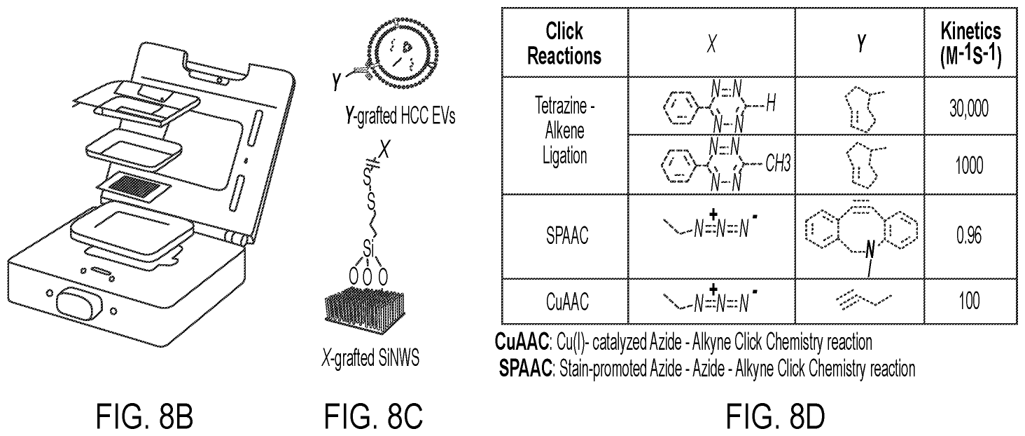

Exploring different Click Chemistry motifs. The Click Chemistry motifs (i.e., Tz and TCO) used in the current version of EV Click Chips demonstrated good HCC EV capture performance. Additional Click Chemistry motifs investigated include methylated Tz/TCO motif, strain-promoted Azide-Alkyne reaction (SPAAC), and Cu(I)-catalyzed Azide-Alkyne reaction (CuAAC) (FIG. 8c/d).

FIGS. 8A-8D are schematics showing various Click Chemistry motifs according to embodiments of the invention. FIG. 8A is a schematic summarizing of the stepwise functional group transformation employed for the preparation of the Tz-grafted SiNWS. FIG. 8B shows the configuration of the proposed new chip holder for housing a Tz-grafted substrate and an overlaid PDMS chaotic mixer. FIG. 8C is a schematic showing new categories of Click Chemistry motifs (i.e., X and Y) will be grafted onto SiNWS and HCC EV capture antibodies, respectively, for achieving ideal balance between EV capture performance and device lifetime. FIG. 8D is a table showing three new different Click Chemistry motif pairs, including tetrazine/Alkene reaction, strain-promoted Azide-Alkyne reaction (SPAAC), and Cu(I)-catalyzed Azide-Alkyne reaction (CuAAC) examined and compared with the original Tz/TCO Click Chemistry reaction.

### References from Example 1

1 Bray, F. et al. Global cancer statistics 2018: GLOBOCAN estimates of incidence and mortality worldwide for 36 cancers in 185 countries. CA Cancer J Clin 68, 394-424, doi:10.3322/caac.21492 (2018).
2 Tsuchiya, N. et al. Biomarkers for the early diagnosis of hepatocellular carcinoma. World J Gastroenterol 21, 10573-10583, doi:10.3748/wjg.v21.i37.10573 (2015).
3 Marrero, J. A. et al. Diagnosis, Staging, and Management of Hepatocellular Carcinoma: 2018 Practice Guidance by the American Association for the Study of Liver Diseases. Hepatology 68, 723-750, doi:10.1002/hep.29913 (2018).
4 Forner, A. & Bruix, J. Biomarkers for early diagnosis of hepatocellular carcinoma. Lancet Oncol 13, 750-751, doi:10.1016/S1470-2045(12)70271-1 (2012).
5 Heitzer, E., Haque, I. S., Roberts, C. E. S. & Speicher, M. R. Current and future perspectives of liquid biopsies in genomics-driven oncology. Nat Rev Genet 20, 71-88, doi:10.1038/s41576-018-0071-5 (2019).
6 Siravegna, G., Marsoni, S., Siena, S. & Bardelli, A. Integrating liquid biopsies into the management of cancer. Nat Rev Clin Oncol 14, 531-548, doi 10.1038/nrclinonc.2017.14 (2017).
7 Krebs, M. G. et al. Molecular analysis of circulating tumour cells-biology and biomarkers. Nat Rev Clin Oncol 11, 129-144, doi:10.1038/nrclinonc.2013.253 (2014).
8 Chen, J. F. et al. Clinical Applications of Nano Velcro Rare-Cell Assays for Detection and Characterization of Circulating Tumor Cells. Theranostics 6, 1425-1439, doi:10.7150/thno.15359 (2016).
9 Dong, J. et al. Nanostructured Substrates for Detection and Characterization of Circulating Rare Cells: From Materials Research to Clinical Applications. Adv Mater, e1903663, doi:10.1002/adma.201903663 (2019).
10 Cristiano, S. et al. Genome-wide cell-free DNA fragmentation in patients with cancer. Nature 570, 385-389, doi:10.1038/s41586-019-1272-6 (2019).
11 Shen, S. Y. et al. Sensitive tumour detection and classification using plasma cell-free DNA methylomes. Nature 563, 579-+, doi:10.1038/s41586-018-0703-0 (2018).
12 Kosaka, N. et al. Exploiting the message from cancer: the diagnostic value of extracellular vesicles for clinical applications. Exp Mol Med 51, 31, doi:10.1038/s12276-019-0219-1 (2019).
13 van Niel, G., D'Angelo, G. & Raposo, G. Shedding light on the cell biology of extracellular vesicles. Nat Rev Mol Cell Biol 19, 213-228, doi:10.1038/nrm.2017.125 (2018).
14 Raposo, G. & Stahl, P. D. Extracellular vesicles: a new communication paradigm? Nat Rev Mol Cell Biol, doi:10.1038/s41580-019-0158-7 (2019).
15 Rahbarghazi, R. et al. Tumor-derived extracellular vesicles: reliable tools for Cancer diagnosis and clinical applications. Cell Commun Signal 17, 73, doi:10.1186/s12964-019-0390-y (2019).
16 Livshits, M. A. et al. Isolation of exosomes by differential centrifugation: Theoretical analysis of a commonly used protocol (vol 5, 17319, 2015). Sci Rep-Uk 6, doi:ARTN 21447 10.1038/srep21447 (2016).
17 Reategui, E. et al. Engineered nanointerfaces for microfluidic isolation and molecular profiling of tumor-specific extracellular vesicles. Nat Commun 9, 175, doi:10.1038/s41467-017-02261-1 (2018).
18 Dong, J. et al. Bio-Inspired Nano Villi Chips for Enhanced Capture of Tumor-Derived Extracellular Vesicles: Toward Non-Invasive Detection of Gene Alterations in Non-Small Cell Lung Cancer. ACS Appl Mater Interfaces 11, 13973-13983, doi:10.1021/acsami.9b01406 (2019).
19 Melo, S. A. et al. Glypican-1 identifies cancer exosomes and detects early pancreatic cancer. Nature 523, 177-182, doi:10.1038/nature14581 (2015).
20 Zhang. P. et al. Ultrasensitive detection of circulating exosomes with a 3D-nanopatterned microfluidic chip. Nat Biomed Eng 3, 438-451, doi:10.1038/s41551-019-0356-9 (2019).
21 Wang. X. et al. Extracellular mRNA detected by molecular beacons in tethered lipoplex nanoparticles for diagnosis of human hepatocellular carcinoma. PLoS One 13, e0198552, doi:10.1371/journal.pone.0198552 (2018).
22 Zhai, W. et al. The spatial organization of intra-tumour heterogeneity and evolutionary trajectories of metastases in hepatocellular carcinoma. Nat Commun 8, 4565, doi:10.1038/ncomms14565 (2017).
23 Hirschfield, H. et al. In vitro modeling of hepatocellular carcinoma molecular subtypes for anti-cancer drug assessment. Exp Mol Med 50, e419, doi:10.1038/emm.2017.164 (2018).
24 Liu, J. P., Dang. H. & Wang. X. W. The significance of intertumor and intratumor heterogeneity in liver cancer. Experimental and Molecular Medicine 50, doi:ARTN e416 10.1038/emm.2017.165 (2018).
25 Court. C. M. et al. A novel multimarker assay for the phenotypic profiling of circulating tumor cells in hepatocellular carcinoma. Liver Transpl 24, 946-960, doi:10.1002/lt.25062 (2018).
26 Wang, S. et al. Three-dimensional nanostructured substrates toward efficient capture of circulating tumor cells. Angewandte Chemie 48, 8970-8973, doi:10.1002/anie.200901668 (2009).
27 Stroock, A. D. et al. Chaotic mixer for microchannels. Science 295, 647-651, doi:10.1126/science.1066238 (2002).
28 Kolb, H. C., Finn, M. G. & Sharpless, K. B. Click Chemistry: Diverse Chemical Function from a Few Good Reactions. Angewandte Chemie 40, 2004-2021, doi:10.1002/1521-3773(20010601)40:11<2004::aidanie2004>3.3.co;2-x (2001).
29 Witt, D. Recent developments in disulfide bond formation. Synthesis-Stuttgart, 2491-2509, doi:10.1055/s-2008-1067188 (2008).
30 Wang, S. et al. Highly efficient capture of circulating tumor cells by using nanostructured silicon substrates with integrated chaotic micromixers. Angewandte Chemie 50, 3084-3088, doi:10.1002/anie.201005853 (2011).
31 Blackman, M. L., Royzen, M. & Fox, J. M. Tetrazine ligation: fast bioconjugation based on inverse-electron-demand Diels-Alder reactivity. Journal of the American Chemical Society 130, 13518-13519, doi:10.1021/ja8053805 (2008).
32 Smith, E. A. et al. Formation, spectroscopic characterization, and application of sulfhydryl-terminated alkanethiol monolayers for the chemical attachment of DNA onto gold surfaces. Langmuir 17, 2502-2507, doi:DOI 10.1021/la001064q (2001).
33 Kalinich, M. et al. An RNA-based signature enables high specificity detection of circulating tumor cells in hepatocellular carcinoma. Proc Natl Acad Sci USA 114, 1123-1128, doi:10.1073/pnas.1617032114 (2017).
34 Wang, S. T. et al. Highly Efficient Capture of Circulating Tumor Cells by Using Nanostructured Silicon Substrates with Integrated Chaotic Micromixers. Angew. Chem. Int. Ed. 50, 3084-3088, doi:10.1002/anie.201005853 (2011).
35 Dong, J. et al. Covalent chemistry on nanostructured substrates enables noninvasive quantification of gene rearrangements in circulating tumor cells. Sci Adv 5, eaav9186, doi:10.1126/sciadv.aav9186 (2019).
36 Sheng, W. et al. Capture, Release and Culture of Circulating Tumor Cells from Pancreatic Cancer Patients Using an Enhanced Mixing Chip. Lab Chip 14, 89-98, doi:10.1039/c3lc51017d (2014).
37 Thery, C., Amigorena, S., Raposo, G. & Clayton, A. Isolation and characterization of exosomes from cell culture supernatants and biological fluids. Current protocols in cell biology Chapter 3, Unit 3.22, doi:10.1002/0471143030.cb0322s30 (2006).
38 Tauro, B. J. et al. Comparison of ultracentrifugation, density gradient separation, and immunoaffinity capture methods for isolating human colon cancer cell line LIM1863-derived exosomes. Methods 56, 293-304, doi:https://doi.org/10.1016/j.ymeth.2012.01.002 (2012).
39 Karver, M. R., Weissleder, R. & Hilderbrand, S. A. Synthesis and evaluation of a series of 1,2,4,5-tetrazines for bioorthogonal conjugation. Bioconjug. Chem. 22, 2263-2270, doi:10.1021/bc200295y (2011).
40 Willms, A. et al. Tumour-associated circulating microparticles: A novel liquid biopsy tool for screening and therapy monitoring of colorectal carcinoma and other epithelial neoplasia. Oncotarget 7, 30867-30875, doi:10.18632/oncotarget.9018 (2016).
41 Julich-Haertel, H. et al. Cancer-associated circulating large extracellular vesicles in cholangiocarcinoma and hepatocellular carcinoma. J Hepatol 67, 282-292, doi:10.1016/j.jhep.2017.02.024 (2017).
42 Llovet, J. M., Bru, C. & Bruix, J. Prognosis of hepatocellular carcinoma: the BCLC staging classification. Semin Liver Dis 19, 329-338, doi:10.1055/s-2007-1007122 (1999).
43 Mazzaferro, V. et al. Liver transplantation for the treatment of small hepatocellular carcinomas in patients with cirrhosis. N Engl J Med 334, 693-699, doi:10.1056/NEJM199603143341104 (1996).
44 Yao, F. Y. et al. Downstaging of hepatocellular cancer before liver transplant: Long-term outcome compared to tumors within Milan criteria. Hepatology 61, 1968-1977, doi:10.1002/hep.27752 (2015).
45 Levine, D. M. et al. Pathway and gene-set activation measurement from mRNA expression data: the tissue distribution of human pathways. Genome Biol 7, R93, doi:10.1186/gb-2006-7-10-r93 (2006).
46 Zhang, D. Y. & Friedman, S. L. Fibrosis-dependent mechanisms of hepatocarcinogenesis. Hepatology 56, 769-775, doi:10.1002/hep.25670 (2012).
47 Blackman, M. L., Royzen, M. & Fox, J. M. Tetrazine ligation: fast bioconjugation based on inverse-electron-demand Diels-Alder reactivity. J. Am. Chem. Soc. 130, 13518-13519, doi:10.1021/ja8053805 (2008).
48 Tzartzeva, K. et al. Surveillance Imaging and Alpha Fetoprotein for Early Detection of Hepatocellular Carcinoma in Patients With Cirrhosis: A Meta-analysis. Gastroenterology 154, 1706-1718 e1701, doi:10.1053/j.gastro.2018.01.064 (2018).
49 Sengupta, S. & Parikh, N. D. Biomarker development for hepatocellular carcinoma early detection: current and future perspectives. Hepat Oncol 4, 111-122, doi:10.2217/hep-2017-0019 (2017).
50 Qu, C. et al. Detection of early-stage hepatocellular carcinoma in asymptomatic HBsAg-seropositive individuals by liquid biopsy. Proc Natl Acad Sci USA 116, 6308-6312, doi:10.1073/pnas.1819799116 (2019).
51 Heitzer, E., Perakis, S., Geigl, J. B. & Speicher, M. R. The potential of liquid biopsies for the early detection of cancer. NPJ Precis Oncol 1, 36, doi:10.1038/s41698-017-0039-5 (2017).
52 Xu, R. H. et al. Circulating tumour DNA methylation markers for diagnosis and prognosis of hepatocellular carcinoma. Nat Mater 16, 1155-1161, doi:10.1038/nmat4997 (2017).
53 Wan, J. C. M. et al. Liquid biopsies come of age: towards implementation of circulating tumour DNA. Nat Rev Cancer 17, 223-238, doi:10.1038/nrc.2017.7 (2017).
54 Ye, Q., Ling, S., Zheng, S. & Xu, X. Liquid biopsy in hepatocellular carcinoma: circulating tumor cells and circulating tumor DNA. Mol Cancer 18, 114, doi:10.1186/s12943-019-1043-x (2019).
55 Peng, K. Q., Yan, Y. J., Gao, S. P. & Zhu, J. Synthesis of large-area silicon nanowire arrays via self-assembling nanoelectrochemistry. Adv Mater 14, 1164-1167, doi:Doi 10.1002/1521-4095(20020816)14:16<1164::Aid-Adma1164>3.0.Co;2-E (2002).

### Example 2

Ewing sarcoma (ES) is a highly aggressive cancer that ranks as the second most frequent bone cancer during childhood and adolescence and is known for frequent metastases and poor prognoses.^{[34]} Recently, ES EVs have been identified to be secreted by ES cells, actively participating in the tumorigenesis, progression, and metastasis of ES by not only reprogramming surrounding normal stromal cells but also promoting intercellular communication within the tumor cells themselves.^{[35,36]} At present, few research efforts focus on isolating ES EVs, likely due to the lack of specific surface biomarkers to target. It is technically challenging to develop an efficient method for isolating ES EVs. As a result, only conventional methods—ultracentrifugation^{[37]} and filtration^{[38]}—have been adopted for their isolation. However, these are incapable of purifying ES EVs out of the non-ES EV background. Recently, an integrated microfluidic digital analysis chip with a dual-probe hybridization assay was developed for the detection of ES-EV mRNA,^{[39]} demonstrating the presence of EWS-rearranged mRNA in ES EVs. However, this platform was not designed for the specific enrichment of ES EVs and is incapable of recovering intact ES EVs for downstream functional study.

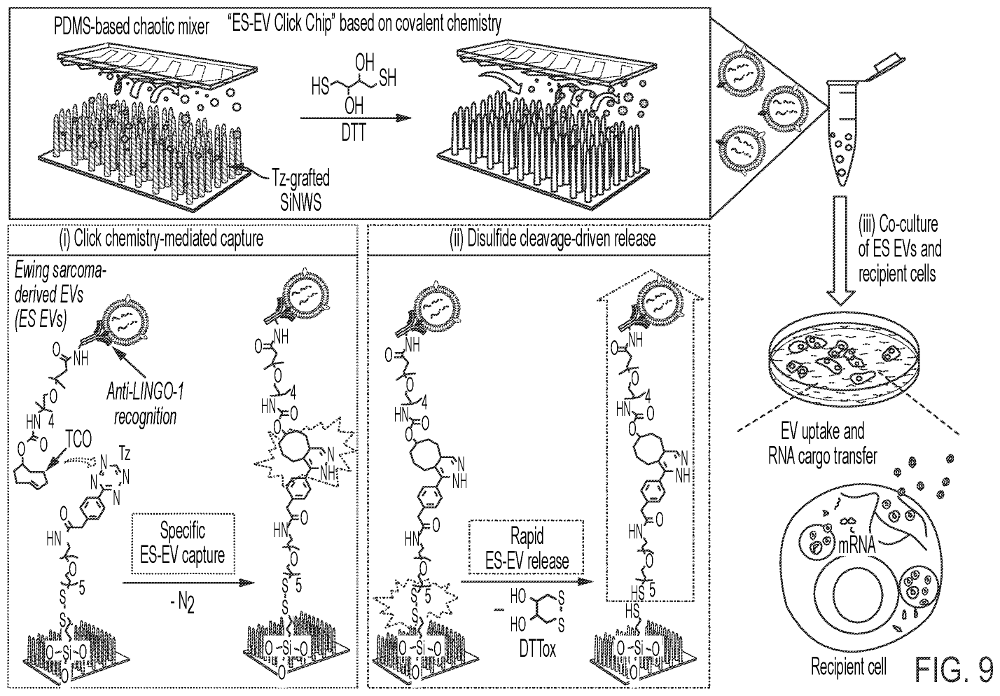

To pave the way for conducting functional studies of ES EVs, a novel ES-EV purification system (i.e., “ES-EV Click Chip”) was introduced by coupling covalent chemistry-mediated EV capture/release within a nanostructure-embedded microchip (FIG. 9). In conjunction with the use of a newly identified ES-specific surface marker, i.e., leucine-rich repeat and immunoglobulin-like domain-containing protein 1 (LINGO-1),^{[40]} ES-EV Click Chip is capable of purifying ES EVs, which can be used for downstream functional studies, i.e., EV uptake and cargo transfer. More specifically, the covalent chemistry-mediated EV capture/release is built upon the combined use of click chemistry^{[41]}-mediated capture of EVs (tagged with anti-LINGO-1 antibody) and subsequent disulfide cleavage^{[42]}-driven EV release. Further, the nanostructures embedded in microchip dramatically increase the device surface area^{[43,44]} contacting/interacting with ES EVs, and the microfluidic chaotic mixer made of polydimethylsiloxane (PDMS) facilitates repeated physical contact^{[45]} between silicon nanowire substrates (SiNWS) and the flow-through ES EVs. In contrast to previous antibody-mediated EV capture,^{[33]} a pair of highly reactive click chemistry motifs,^{[46]} i.e., tetrazine (Tz) and trans-cyclooctene (TCO), were grafted onto the embedded SiNWS (via surface modification) and ES EVs (via TCO-antibody conjugation), respectively. Subsequently, the inverse electron demand Diels-Alder (IEDDA) cycloaddition^{[47]} between Tz and TCO with a rate constant^{[48]} of ˜10⁴ M⁻¹ S⁻¹ mediates the rapid, chemoselective, and irreversible capture of TCO-anti-LINGO-1-grafted ES EVs onto Tz-grafted SiNWS with improved capture efficiency and reduced background. After click chemistry-mediated ES-EV capture, exposure to a disulfide cleavage agent—1,4-dithiothreitol (DTT)^{[49]}—leads to the prompt release of the ES EVs from the SiNWS by breaking the embedded disulfide bond. ES-EV Click Chips were utilized to purify ES EVs that are genetically characterized by harboring specific chromosomal translocations. These translocations generate fusions of EWSR1 to one of the ETS gene family members, including FLI1 (90-95%) and ERG (5-10%).^{[50]} providing specific molecular markers for detecting ES EVs.^{[51]} By using immunogold-transmission electron microscopy (TEM), it was confirmed that LINGO-1 is expressed on the surface of ES EVs. The anti-LINGO-1 antibody is exploited to recognize ES EVs in ES-EV Click Chip purification system. ES-EV Click Chips are shown to efficiently purify ES EVs without any size bias, including exosomes, microvesicles, and oncosomes. Moreover, it is demonstrated that the recovered ES EVs have well-preserved viability and RNA cargo contents, and can be used in downstream functional studies, i.e., EV uptake and RNA cargo transfer, which are essential for exploring their physiological and pathological functions in intercellular communication.

FIG. 9 is a schematic illustration of “ES-EV Click Chip” and its working mechanism based on covalent chemistry-mediated purification of ES EVs. (i) Click chemistry (between Tz and TCO)-mediated specific EV capture in the presence of the LINGO-1 antibody; (ii) disulfide cleavage-driven rapid EV release upon exposure to DTT; (iii) subjecting the purified ES EVs to downstream functional studies, i.e., EV uptake and RNA cargo transfer.

### 2. Results and Discussions

### 2.1. Identification of LINGO-1 as a Specific Surface Marker of ES EVs

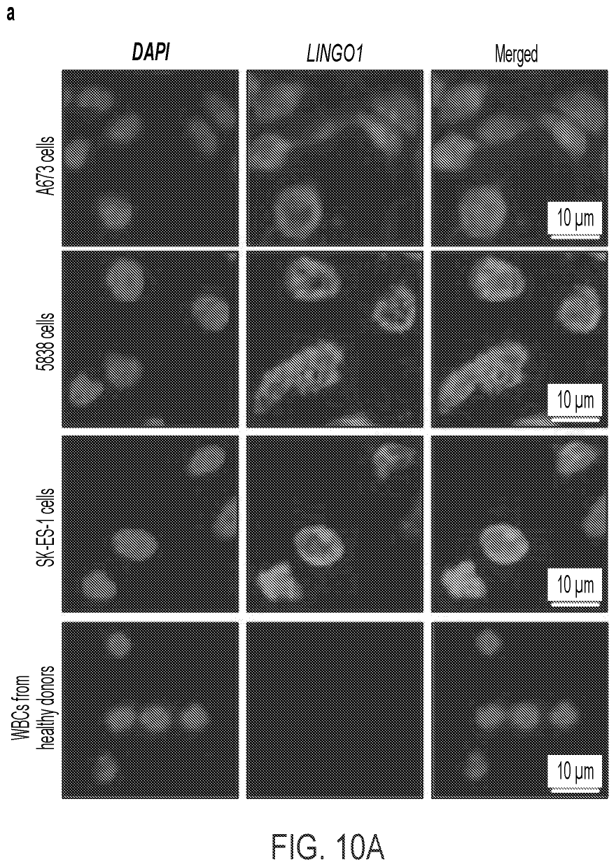

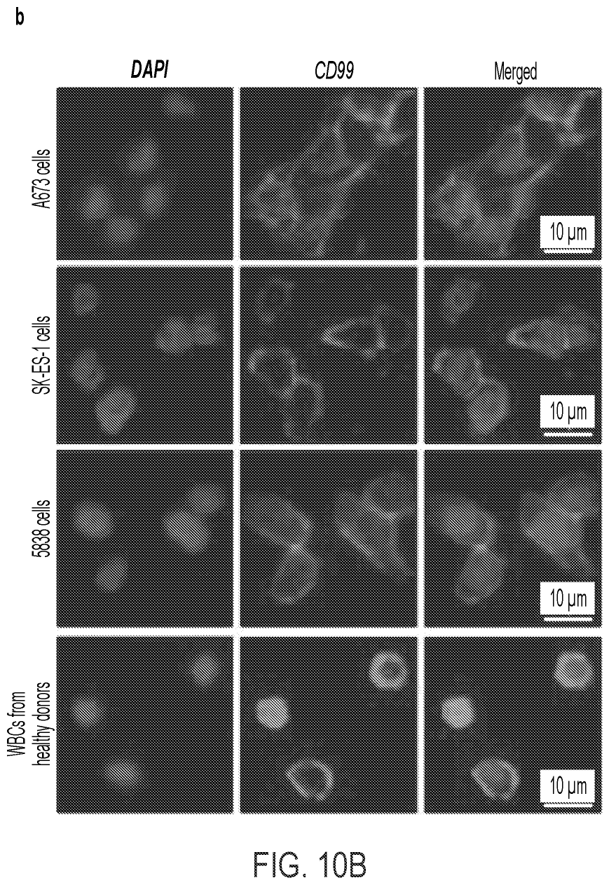

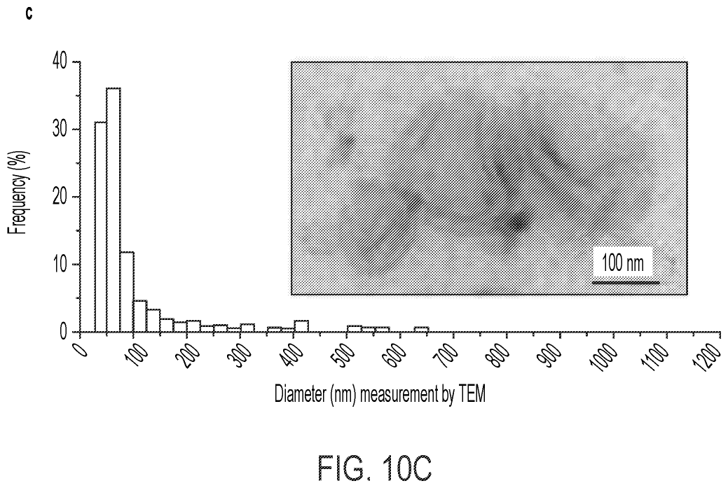

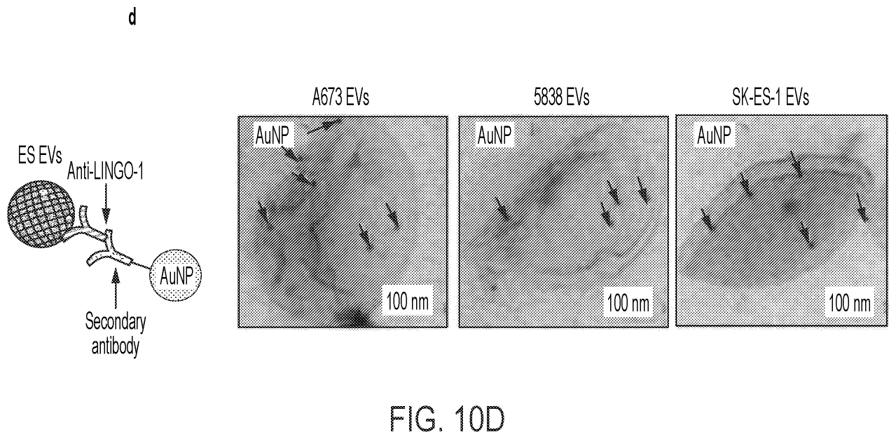

To determine the specificity of LINGO-1 (a transmembrane signaling protein considered as a new marker and therapeutic target expressed on ES tumor surface^{[40]}) as an ES cell surface marker, immunofluorescence staining was used to evaluate the expression of LINGO-1 on ES cell lines (e.g., A673, ES-5838, and SK-ES-1 cell lines), and white blood cells (WBCs) isolated from healthy donors' blood. For comparison, expression of CD99 (a transmembrane glycoprotein commonly used as an ES cell surface marker^{[52,53]}) on ES cell lines and WBCs was also evaluated. The fluorescent images (FIG. 10A) showed that LINGO-1 was a specific surface marker expressed on all the three ES cell lines but not on WBCs, while CD99 was not specific for ES cells as it was also expressed on WBCs (FIG. 10B). Then, ES EVs were isolated from the serum-free medium of ES cells, i.e., A673 cells (female origin, harboring EWS-FLI1 type 1 rearrangement), ES-5838 cells (male origin, harboring EWS-ERG rearrangement), and SK-ES-1 cells (male origin, harboring EWS-FLI1 type 2 rearrangement), by ultracentrifugation.^{[17]} TEM characterization showed that ES EVs had a cup or spherical-shaped morphology (FIG. 10C). They had a diameter range of 30-1150 nm, of which 89% ranged in diameter of 30-150 nm. Then immunogold-TEM was employed to detect LINGO-1 expression on ES EVs. As shown in FIG. 10D, A673 EVs, ES-5838 EVs, and SK-ES-1 EVs were all labeled with multiple 10 nm gold nanoparticles (AuNPs) via mouse anti-LINGO-1 and goat anti-mouse IgG H&L 10 nm AuNPs. These results demonstrate the expression of LINGO-1 on the surface of ES EVs.

FIGS. 10A-10D are microscopic images and graphs showing detection of LINGO-1 expression on ES cell lines and ES EVs according to an embodiment of the invention. FIG. 10A shows immunofluorescence images showing that LINGO-1 was expressed on the cell plasma membrane of A673 cells, ES-5838 cells, and SK-ES-1 cells, with a granular pattern, but not on WBCs isolated from healthy donors' blood. Nuclei of cells were stained with DAPI. FIG. 10B shows immunofluorescence images showing that CD99 was expressed on the cell membrane of A673 cells, ES-5838 cells, and SK-ES-1 cells, as well as WBCs, with a linear pattern. Nuclei of cells were stained with DAPI. FIG. 10C is a graph showing size distribution of ES EVs (n=886) measured by TEM. Inset is a TEM image of ES EVs. FIG. 10D shows a schematic diagram (left) and TEM images (right) for detecting LINGO-1 expression on ES EVs, including A673 EVs, ES-5838 EVs, SK-ES-1 EVs, using immunogold staining of LINGO-1. The arrows point to the AuNPs labeled on the ES EVs.

### 2.2. Fabrication of ES-EV Click Chip Purification System

ES-EV Click Chip is composed of two components: (i) a Tz-grafted SiNWS and (ii) a PDMS-based chaotic mixer with a serpentine microchannel. Si nanowires with diameters of 100-200 nm and lengths of 3-5 or 7-10 μm were fabricated via a combination of photolithographic patterning and silver (Ag) nanoparticle-templated wet etching.^{[54]} The densely packed Si nanowires (spacings=200-400 nm) provide large surface areas for immobilizing Tz moieties. Through a 3-step modification process^{[55]}: (i) vapor deposition of (3-mercaptopropyl) trimethoxysilane (MPS), (ii) incorporation of a disulfide linker via ortho-pyridyl disulfide polyethylene glycol amine (OPSS-PEG-NH₂), and (iii) NHS ester reaction between Tz-sulfo-NHS ester and the terminal primary amine group on SiNWS, abundant Tz moicties were tethered onto the Si nanowires to generate the Tz-grafted SiNWS. PDMS-based chaotic mixers were fabricated with herringbone patterns by inductively coupled plasma-reactive ion etching (ICP-RIE).^{[45,56]} The herringbone pattern spacings and microchannel widths/lengths/heights (2 mm×60 mm×70 μm) were configured to facilitate direct physical contact^{[56]} between the functional SiNWS and EVs. Finally, a microfluidic chip holder was used to combine the PDMS-based chaotic mixer with the Tz-grafted SiNWS to make a complete ES-EV Click Chip, and an automated fluidic handler was employed to handle EV samples.

Prior to EV capture studies, the complementary click chemistry moieties—TCO—were conjugated^{[57]} onto goat anti-LINGO-1 via the NHS ester reaction between TCO-PEG₄-NHS ester and the primary amine groups on anti-LINGO-1 to produce TCO-anti-LINGO-1 conjugate. As a model system for testing the EV-capture/release performance of ES-EV Click Chips, A673 EV samples were prepared by homogeneously re-suspending A673 EV pellets into serum-free medium and divided into several replicates (each 100 μL). The TCO-anti-LINGO-1 conjugate was pre-incubated with A673 EV samples to allow the specific antigen-antibody interaction. Then the obtained TCO-anti-LINGO-1-grafted A673 EV sample was run through ES-EV Click Chip, resulting in the efficient, chemoselective, and irreversible capture of A673 EVs on the Tz-grafted SiNWS via the IEDDA cycloaddition^{[46]} between Tz and TCO moicties. Afterward, to release the captured EVs, DTT (50 mM, 50 μL) was injected into ES-EV Click Chip. DTT-mediated thiol-disulfide exchange reactions cause the reduction and cleavage of the disulfide bonds linking ES EVs or spare Tz moicties to SiNWS, resulting in the prompt release of captured ES EVs from the SiNWS.

### 2.3. Characterization of the ES-EV Capture and Release Process

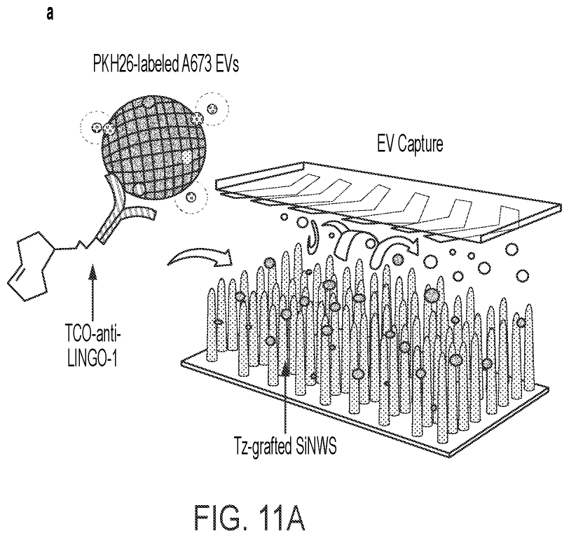

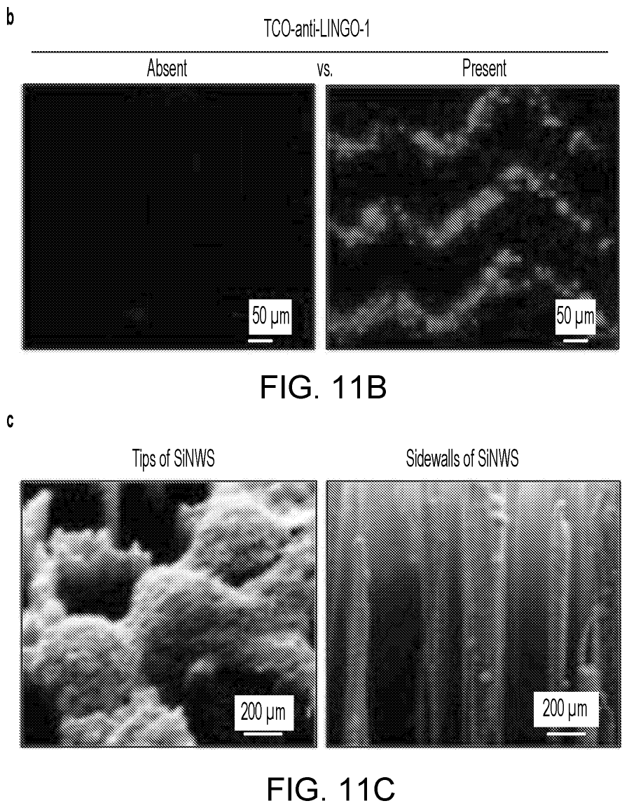

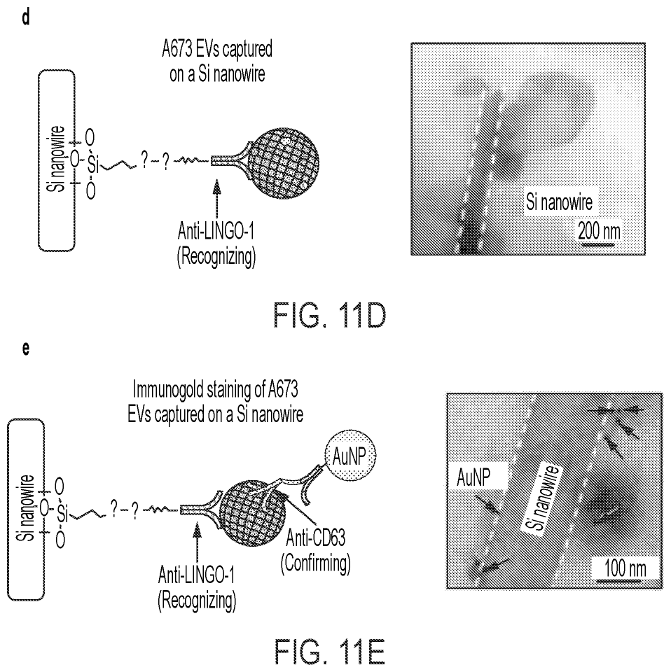

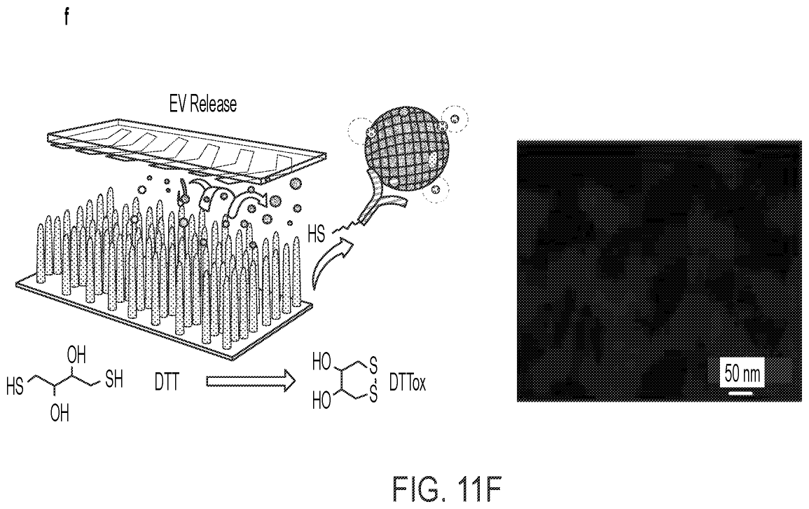

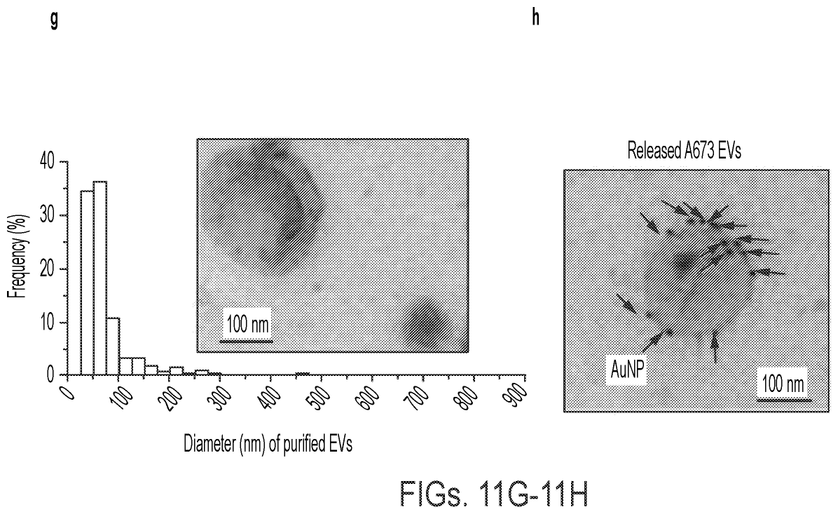

To demonstrate the feasibility of click chemistry-mediated EV capture on SiNWS, PKH26 red-fluorescent dye was used to label A673 EVs (FIG. 11A). The PKH26-labeled A673 EV samples were injected into the ES-EV Click Chips in the absence and presence of TCO-anti-LINGO-1 conjugate, respectively. After EV capture, SiNWS were disassembled from ES-EV Click Chips and observed by fluorescence microscopy. The dimmer appearance of SiNWS under fluorescence microscopy (FIG. 11B) shows that the PKH26-labeled A673 EVs were unable to be captured on Tz-grafted SiNWS without TCO-LINGO-1 conjugate. In comparison, numerous conspicuous red fluorescent spots, i.e., PKH26-labeled A673 EVs, were captured on Tz-grafted SiNWS in the presence of TCO-anti-LINGO-1 conjugate and distributed evenly in a herringbone pattern. These results demonstrate that ES-EV Click Chips capture ES EVs in a TCO-LINGO-1 dependent manner. Next, to observe the EV distribution on Tz-grafted SiNWS with higher resolution, scanning electron microscopy (SEM) was employed to characterize the tops and cross-sections of Si nanowire arrays. As shown in FIG. 11C, both tips and sidewalls of Si nanowires had immobilized A673 EVs. Besides, Si nanowires with captured A673 EVs were mechanically detached from the substrate for TEM characterization. FIG. 11D shows a TEM image of a single Si nanowire with various sizes of EVs captured on its tip and sidewalls. The results of EV distribution along the Si nanowires were consistent with the previous observations of Nano Villi Chips.^{[33]} Then, immunogold-TEM was used to detect the expression of CD63 (tetraspanins, a surface marker for EVs, preferentially small EVs^{[58]}) on the captured A673 EVs. As shown in FIG. 11E, EVs captured on a Si nanowire were successfully labeled with multiple 10 nm AuNPs. These results suggest that ES-EV Click Chips can effectively capture tumor-derived EVs without size bias and structural damage.

To characterize the release process of ES EVs, DTT was injected into the ES-EV Click Chip, which had captured PKH26-labeled A673 EVs. As shown in FIG. 11F, DTT resulted in the immediate diminution of the red fluorescence, indicating that the captured A673 EVs could be effectively released from the SiNWS. TEM characterization showed that the purified A673 EVs had a cup or spherical-shaped morphology and diameters of 30-900 nm, of which 87% ranged in diameter of 30-150 nm (FIG. 11G). The size distribution of the purified A673 EVs was also measured using dynamic light scattering (DLS). The recovered A673 EVs had a size distribution similar to that of A673 EVs before capture. Moreover, the purified A673 EVs could also be labeled with multiple 10 nm AuNPs via immunogold staining of CD63 (FIG. 11H), showing the integrity of the purified ES EVs.

FIGS. 11A-11H are microscopic images and schematics showing characterization of the ES-EV capture and release process according to an embodiment of the invention. A) Schematic illustration showing that click chemistry-mediated capture of PKH26-labeled A673 EVs on ES-EV Click Chips in the presence of TCO-anti-LINGO-1 conjugate. B) Fluorescence images of PKH26-labeled A673 EVs (red fluorescent spots) that were immobilized on Tz-grafted SiNWS in the absence (left image) and presence (right image) of TCO-LINGO-1 conjugate. C) SEM images displaying A673 EVs captured on the tips of Si nanowire arrays (left image) and the sidewalls of Si nanowires (right image). D) Schematic diagram (left) and TEM image (right) showing A673 EVs immobilized on a detached Si nanowire. E) Schematic representation (left) and TEM image (right) illustrating the immunogold staining for the detection of CD63 expression on the A673 EVs captured on a Si nanowire. F) Schematic diagram (left) and fluorescence image (right) showing the DTT-mediated release of PKH26-labeled EVs from ES-EV Click Chip, resulting in the immediate diminution of PKH26-labeled A673 EVs (red fluorescent spots) on the SiNWS. G) Size distributions of A673 EVs (n=615) purified by ES-EV Click Chips. Inset is a TEM image of purified A673 EVs. H) Immunogold-TEM image showing the expression of CD63 on the purified A673 EVs, indicating the integrity of purified ES EVs.

### 2.4. Optimization of EV Capture/Release Performance of ES-EV Click Chips

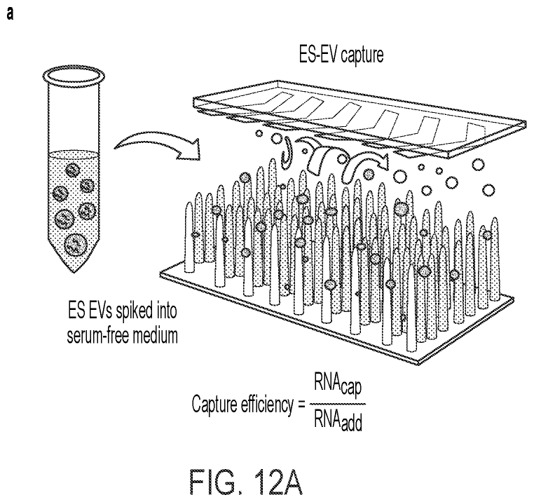

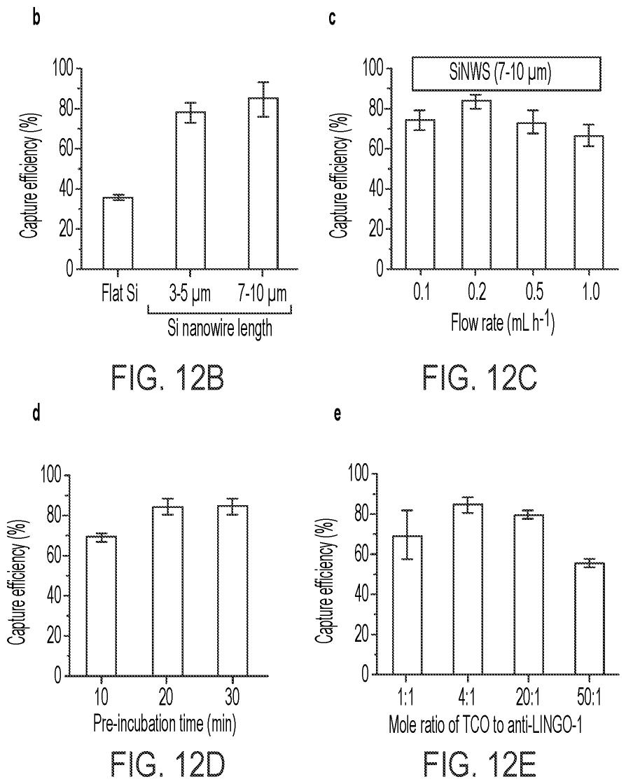

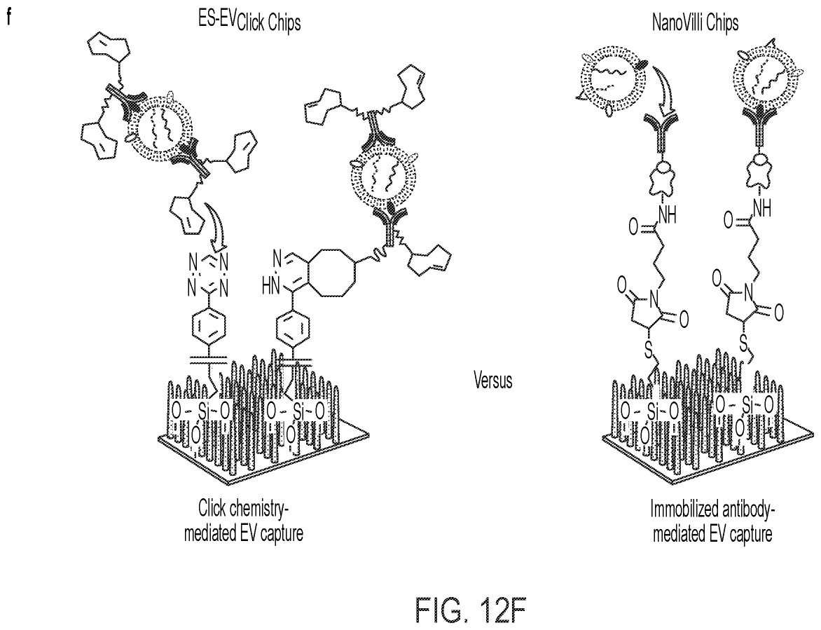

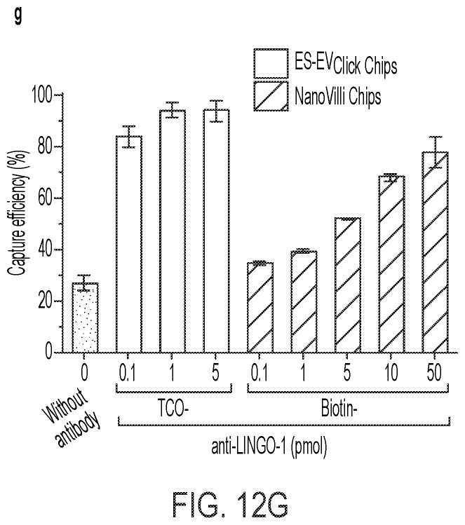

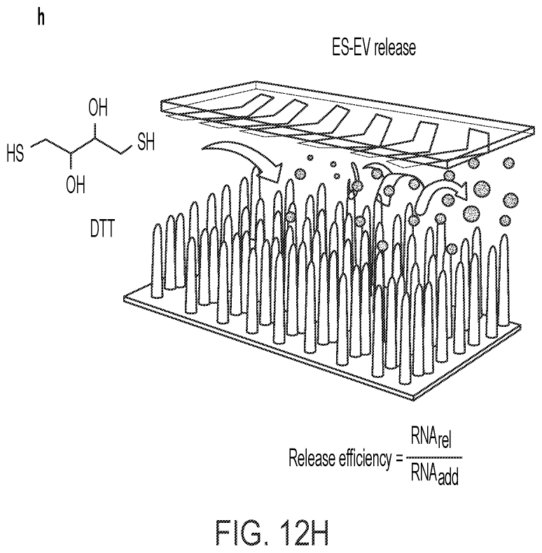

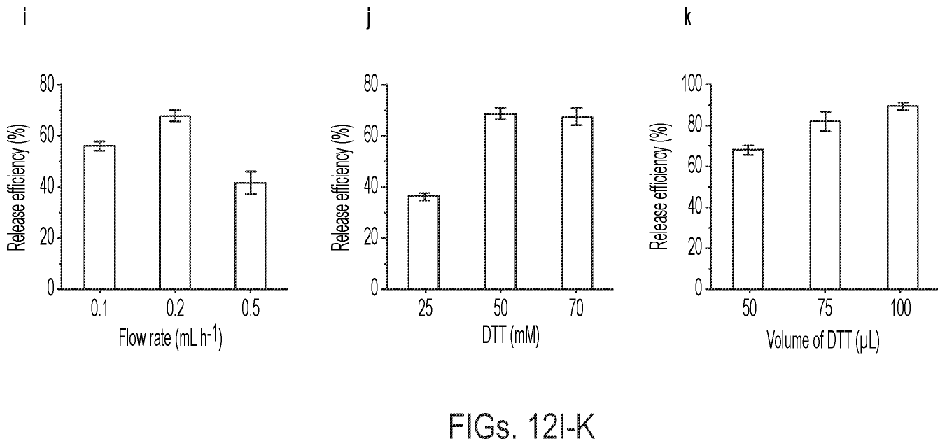

To optimize the ES-EV capture performance of ES-EV Click Chip (FIG. 12A), TCO-anti-LINGO-1 grafted A673 EV samples were injected into ES-EV Click Chips and extracted EV-derived RNA by introducing 700 μL of QIAzol lysis reagent (Qiagen, USA). Subsequently, RNA was purified with miRNeasy Micro Kits (Qiagen, USA) and quantified by Qubit RNA HS Assay using a Qubit 3.0 Fluorometer. The ES-EV capture efficiency was evaluated by calculating the mass fraction of the RNA extracted from the captured ES EVs (RNA_{cap}) compared to the RNA extracted from the initially added ES EVs (RNA_{add}). The effects of different experimental parameters, such as Si nanowire substrate, flow rate, pre-incubation time of TCO-anti-LINGO-1 and ES-EV samples, were evaluated and concentration of TCO-anti-LINGO-1, on ES-EV capture efficiency. The influence of Si nanowire length (0, 3-5, and 7-10 μm) on ES-EV capture efficiency was shown in FIG. 12B. With the increase of Si nanowire length, the ES-EV capture efficiency increased from 36% (Si nanowire=0 μm) to 84% (Si nanowire=7-10 μm) in the presence of 0.1 pmol of TCO-anti-LINGO-1 with a flow rate of 0.2 mL h⁻¹. SEM characterization and dissipative particle dynamics (DPD) simulation^{[59]} were used in the previous development of Nano Villi Chips^{[33]} to study the EV capture process by Si nanowire matrix and demonstrated that a total Si nanowire length of 10 μm was enough to enhance the capture of tumor-derived EVs. Therefore, 7-10 μm Si nanowire was used in the subsequent studies. Next, the effects of flow rates of 0.1, 0.2, 0.5, and 1.0 mL h⁻¹ on ES-EV capture efficiency of ES-EV Click Chips was evaluated. The results in FIG. 12C showed that the optimal flow rate was 0.2 mL h⁻¹. consistent with the previously reported Nano Villi Chips.^{[33]} Pre-incubation time of TCO-LINGO-1 conjugate and A673 EV samples was evaluated and it was found that 20 min was enough to obtain a satisfactory capture efficiency (FIG. 12D). Prolonging pre-incubation time to 30 min had no significant benefit. The effect of the TCO-to-anti-LINGO-1 mole ratio on EV-capture efficiency was also tested with a result of the optimal mole ratio of 4:1 (FIG. 12E).

Then the effects of different concentrations of anti-LINGO-1 conjugates on the capture efficiencies of ES-EV Click Chips and Nano Villi Chips was compared. The schematic diagram of FIG. 12F illustrates the different EV capture mechanisms of ES-EV Click Chips (i.e., click chemistry-mediated EV capture) and the previously reported Nano Villi Chips (i.e., immobilized antibody-mediated EV capture). According to the results shown in FIG. 12G, only 27% of ES EVs were captured on ES-EV Click Chips in the absence of anti-LINGO-1. while 84% of ES EVs were captured on ES-EV Click Chips in the presence of 0.1 pmol of TCO-anti-LINGO-1. These results show that ES EVs are captured in a LINGO-1 dependent manner. The EV capture efficiency of ES-EV Click Chips was up to maximum efficiency of 94% in the presence of 1 pmol of TCO-anti-LINGO-1. A higher quantity of TCO-anti-LINGO-1 did not further increase EV-capture efficiency. Besides, ES-EV Click Chips had advantages of high efficiency and low antibody consumption over Nano Villi Chips, as Nano Viili Chips need 500 times more biotin-anti-LINGO-1 (50 pmol) to achieve a capture efficiency of 78%. These can be attributed to the rapid, chemoselective, and irreversible click chemistry-mediated capture mechanism, as well as the significantly increased number of click reaction sites between TCO moieties grafted on EVs and Tz moicties functionalized on Si nanowire arrays, leading to the more efficient ES-EV immobilization on ES-EV Click Chips than Nano Villi Chips. Furthermore, the capture capacity of ES-EV Click Chips was examined by introducing different amounts of A673 EVs into the devices, followed by the quantification of EV-derived RNA. The ES-EV Click Chips were saturated after capturing a quantity of A673 EVs, which were lysed to obtain 200 ng of EV-derived RNA.

After EV capture, DTT solution was injected into ES-EV Click Chips to release ES EVs from the SiNWS (FIG. 12H). The EV release efficiency was calculated as the mass fraction of RNA extracted from the released ES EVs (RNArel) compared to the RNA extracted from the initially added ES EVs (RNA_{add}). 50 μL of DTT (50 mM) was used to release ES EVs that were captured on the chips and evaluated the influence of flow rate (0.1. 0.2, and 0.5 mL h⁻¹) on the EV release efficiency. As shown in FIG. 12I, a flow rate of 0.2 mL h⁻¹ enabled the captured EVs to be released in 15 min with a maximum efficiency of 68%. Neither a lower nor a higher flow rate increased EV release significantly. Next, the effect of concentration (25, 50, and 70 mM) of DTT solution (50 μL) on EV release efficiency (FIG. 12J) was evaluated. At a flow rate of 0.2 mL h⁻¹, after DTT concentration increased to 50 mM, a more concentrated DTT solution (70 mM) with a defined DTT volume and exposure time did not significantly improve EV release efficiency. Then the volume of DTT (50 mM) solution was increased in order to improve the EV-release efficiency via the increase in DTT amount and exposure time. Results summarized in FIG. 12K showed that the ES-EV release efficiency increased gradually with the increasing volume of DTT, up to maximum efficiency of 90% when 100 μL of DTT (50 mM) ran through ES-EV Click Chips. Overall, these results indicate that the ES EVs captured by ES-EV Click Chips can be effectively released by DTT-driven disulfide cleavage.

FIGS. 12A-12K are schematics and graphs showing evaluation and optimization of EV-capture/release performance of ES-EV Click Chips according to an embodiment of the invention. FIG. 12A is a schematic diagram depicting that ES EVs (spiked into the serum-free medium) are subjected to ES-EV Click Chip for evaluating the EV-capture efficiency. FIG. 12B shows EV-capture efficiencies using different substrates. FIG. 12C shows effect of the flow rate on EV-capture efficiency (Si nanowire length=7-10 μm, TCO-anti-LINGO-1=0.1 pmol). FIG. 12D shows the effect of the pre-incubation time of TCO-LINGO-1 conjugate and EV samples on EV-capture efficiency. FIG. 12E shows the effect of TCO-to-anti-LINGO-1 mole ratio on EV-capture efficiency. FIG. 12F is a schematic illustrating EV capture mechanisms of ES-EV Click Chips and Nano Villi Chips, respectively. FIG. 12G shows EV capture efficiencies of ES-EV Click Chips and Nano Villi Chips with different amounts of TCO-anti-LINGO-1 and biotin-anti-LINGO-1, respectively. EV capture efficiency without any antibody was also evaluated on ES-EV Click Chips as a control. FIG. 12H is a schematic diagram illustrating that DTT is injected into ES-EV Click Chips for evaluating EV-release efficiency. FIG. 121 shows the effect of the flow rate on EV-release efficiency with 50 μL of DTT (50 mM). FIG. 12J shows concentration optimization of DTT solution (50 μL) for EV release. K) The increase of DTT (50 mM) volume for improving the EV-release efficiency.

2.5. Detection of EWS Rearrangements in ES EVs by Coupling ES-EV Click Chips with Reverse Transcription Droplet Digital PCR

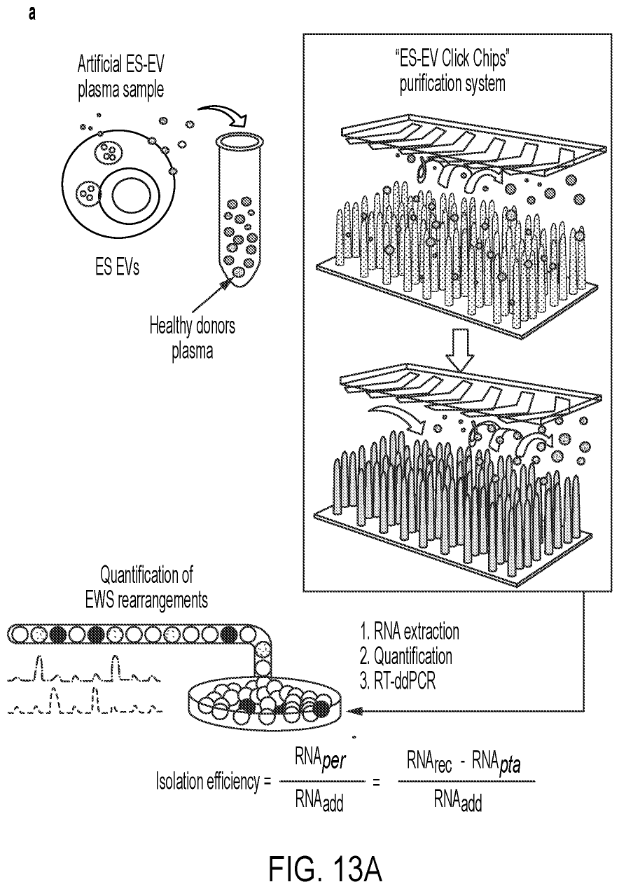

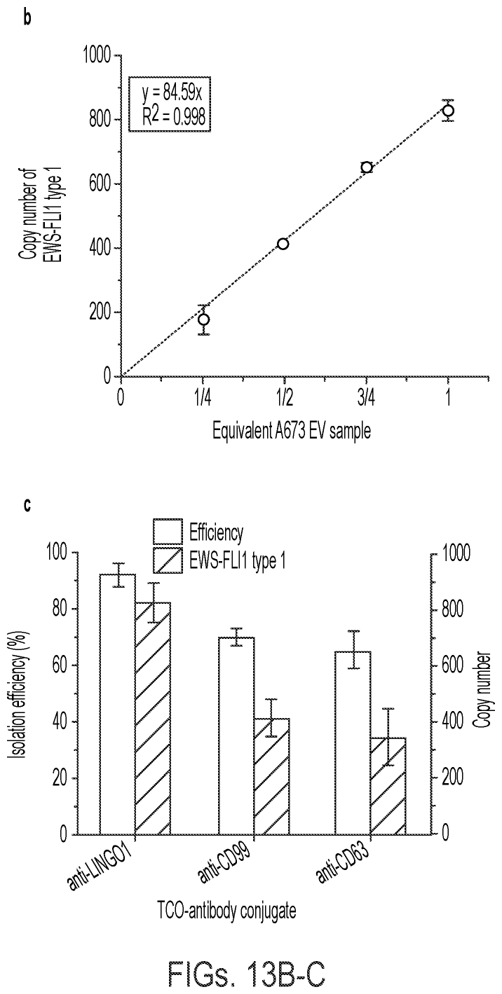

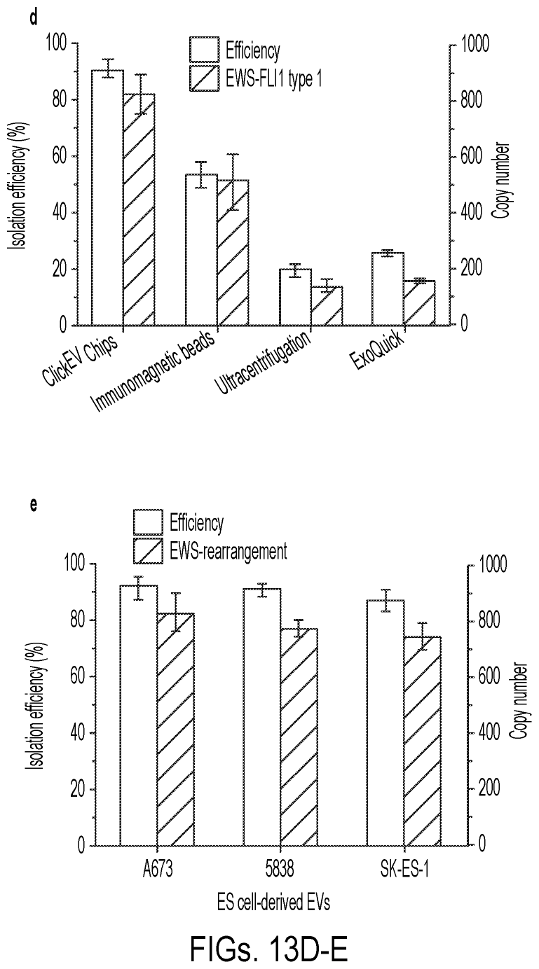

To demonstrate the feasibility of detecting EWS rearrangements using reverse transcription droplet digital PCR (RT-ddPCR) in ES EVs purified by the ES-EV Click Chips, artificial ES-EV plasma samples were prepared by homogeneously re-suspending ES EV pellets into healthy donors' blood plasma (containing a significant quantity of normal cell-derived EVs) and divided into several replicates (each 100 μL). As illustrated in FIG. 13A, ES-EV plasma samples were purified by ES-EV Click Chips, and RNA was extracted from the purified ES EVs and subjected to downstream RT-ddPCR for the quantification of EWS rearrangements. The RNA extracted from the purified ES EVs (RNAₚᵤᵣ) was obtained by subtracting the RNA extracted from the background plasma (RNAₚₗₐ) from the total recovered RNA (RNA_{rec}). Then, the isolation efficiency was calculated as the mass fraction of RNAₚᵤᵣ compared to the RNA extracted from the initially added ES EVs (RNA_{add}). The specificity was evaluated through the copy number of EWS rearrangements, which are specific molecular markers of ES and have a linear correlation (y=18.56x, R²=0.998) with the amount of ES EV-derived RNA. FIG. 13B shows that there is a positive linear correlation between the amount of artificial A673 EV plasma samples and the detected copy number of EWS-FLI1 type 1 rearrangement after ES-EV Click Chip purification.

Then, the isolation efficiency and specificity of ES-EV Click Chips was compared using 1 pmol of TCO-anti-LINGO-1, TCO-anti-CD99, and TCO-anti-CD63 conjugates, because CD99 had been used as an ES cell surface marker to isolate circulating tumor cells^{[52,53]} and CD63 was used as a surface marker to isolate EVs (preferentially small EVs^{[58]}). As shown in FIG. 13C, TCO-anti-LINGO-1 had the highest isolation efficiency of 91% and detected 819 copies of EWS-FLI1 type 1 rearrangement in comparison with TCO-anti-CD99 (70%, 416 copies) and TCO-anti-CD63 (65%, 347 copies). Considering the fact that CD99 and CD63 are ubiquitously expressed on all EVs (i.e., ES EVs and background EVs in plasma samples), when the same quantity (1 pmol) of either TCO-anti-CD99 or TCO-anti-CD63 was added to the plasma sample, only a portion of ES EVs in the plasma sample was labeled with antibody, leading to the low recovery rates of ES EVs. In contrast, the small quantity (1 pmol) of TCO-anti-LINGO-1 (highly specific to ES EVs) is sufficient to label the ES EVs in the plasma sample, resulting in the high recovery rates of ES EVs. Therefore, anti-LINGO-1 is superior to anti-CD99 and anti-CD63 in the efficiency and specificity of purifying ES EVs with ES-EV Click Chips. The coefficient of variation (CV)% of isolation efficiency calculated from five independent tests was 4.0%, representing the ES-EV purification reproducibility of ES-EV Click Chips.

The isolation performance of ES-EV Click Chips was compared to immunomagnetic beads^{[60]} and ultracentrifugation^{[37]} (two commonly used EV enrichment methods), as well as the ExoQuick ULTRA EV Isolation Kit for Serum and Plasma (non-specifically isolating total EVs using an EV precipitation mechanism). The results summarized in FIG. 13D demonstrate that the efficiencies of isolating A673 EVs and copy numbers of EWS-FLI1 type 1 rearrangement of immunomagnetic beads (54%, 512 copies), ultracentrifugation (20%, 144 copies) and the ExoQuick Kit (26%, 156 copies) were significantly lower than that of ES-EV Click Chips (91%, 819 copies). Finally, the general applicability of ES-EV Click Chips for purifying different ES EVs, including A673 EVs, ES-5838 EVs (harboring EWS-ERG rearrangement), and SK-ES-1 EVs (harboring EWS-FLI1 type 2 rearrangement) was examined. As summarized in FIG. 13E, the chips achieved the efficient purification of ES-5838 EVs (90%, 762 copies) and SK-ES-1 EVs (86%, 731 copies). Altogether, these results show that ES-EV Click Chips have excellent performance for purifying ES EVs and enable the quantification of their specific molecular markers—EWS rearrangements. To demonstrate the potential clinical application of ES-EV Click Chips, four EWS rearrangement positive ES patients were recruited [confirmed by fluorescence in situ hybridization (FISH)] and collected their plasma samples for this feasibility study. Control studies were performed on four healthy donors (HDs) in parallel. For each study, 0.3 mL of plasma sample was run through the ES-EV Click Chip under the optimal condition. After extracting RNA from the purified ES EVs, EWS rearrangements were successfully detected (copy number range from 35 to 216) using RT-ddPCR. All of four HDs were negative for EWS rearrangements. These results showed that ES-EV Click Chips can potentially be used for non-invasive detection of EWS rearrangements for ES patients.

FIGS. 13A-13E are schematics and graphs showing detection of EWS rearrangements in ES EVs by coupling ES-EV Click Chips with RT-ddPCR according to an embodiment of the invention. FIG. 13A is a schematic depicting the general workflow of ES-EV Click Chips to evaluate isolation efficiency and detect EWS rearrangements using artificial ES-EV plasma samples. FIG. 13B is a graph showing linear correlation between the amount of artificial A673 EV plasma samples and the detected copy number of EWS-FLI1 type 1 rearrangement after ES-EV Click Chip purification. FIG. 13C is a comparison of the isolation efficiency and specificity of ES-EV Click Chips using 1 pmol of TCO-anti-LINGO-1, TCO-anti-CD99, and TCO-anti-CD63 conjugates. FIG. 13D is a comparison of the isolation efficiency and specificity of ES-EV Click Chips, immunomagnetic beads, ultracentrifugation, and ExoQuick ULTRA EV Isolation Kit using artificial A673 EV plasma samples. FIG. 13 shows the general applicability of ES-EV Click Chips for purifying different ES EVs, including A673 EVs (harboring EWS-FLI1 type 1 rearrangement), ES-5838 EVs (harboring EWS-ERG rearrangements), and SK-ES-1 EVs (harboring EWS-FLI1 type 2 rearrangement), from artificial plasma samples.

### 2.6. Downstream Functional Studies of the Recovered ES EVs

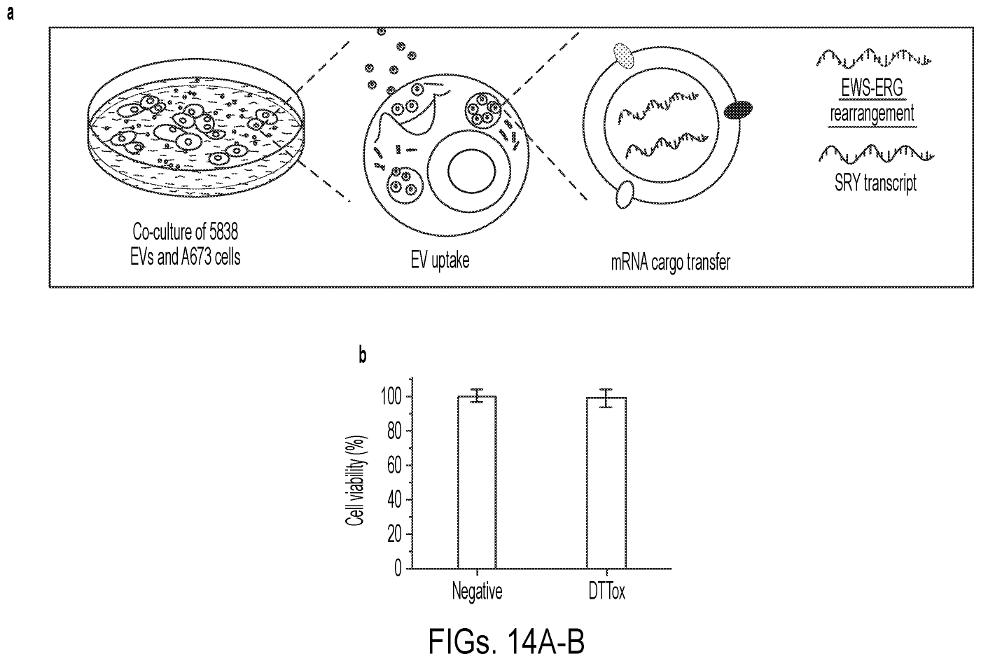

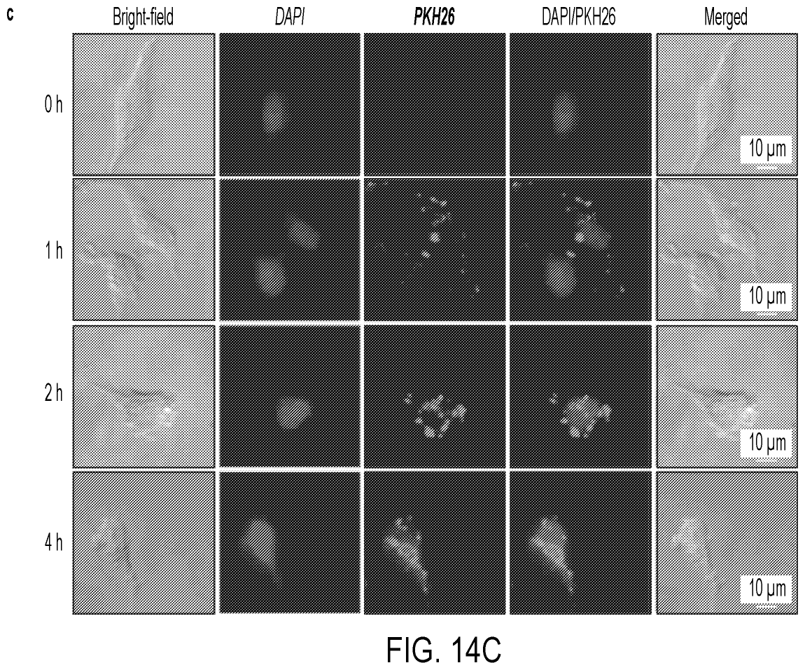

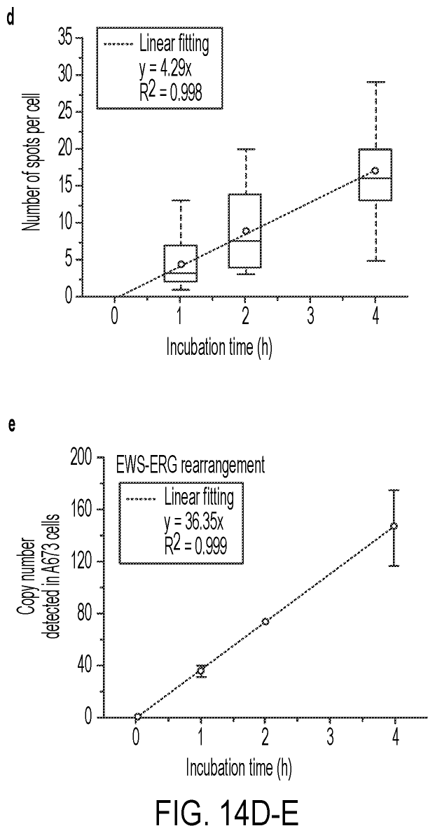

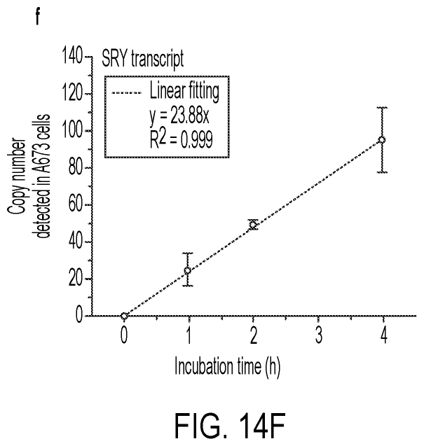

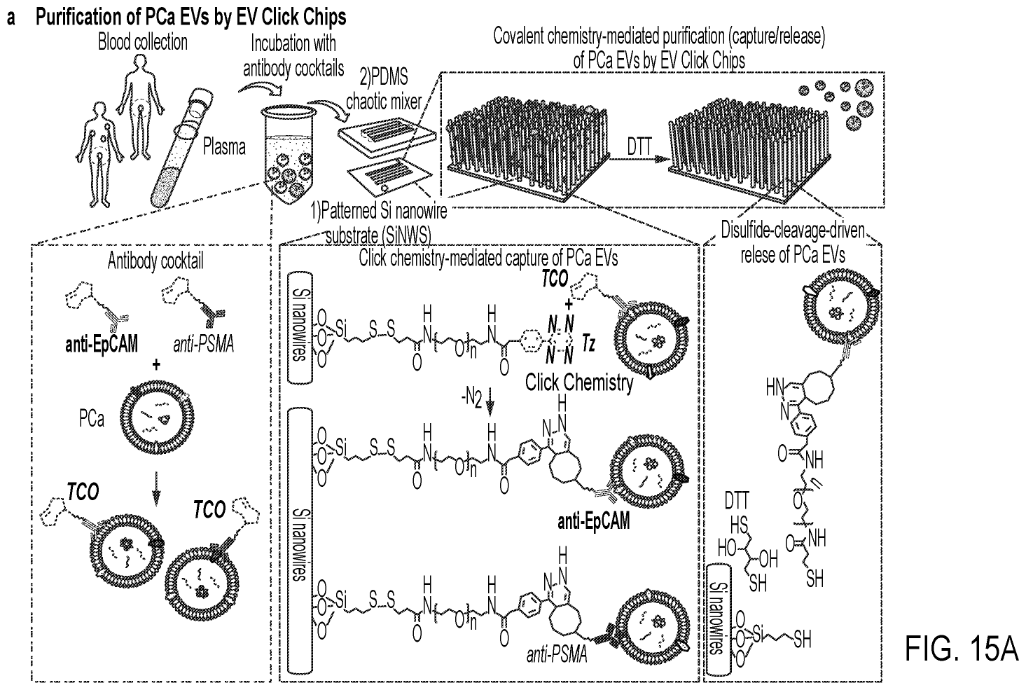

The purified ES EVs can be co-cultured with recipient cells and studied for EV uptake and RNA cargo transfer (FIG. 14A). Several studies^{[61,62]} have shown that DTTox (i.e., trans-4,5-dihydroxy-1,2-dithiane, a nontoxic intramolecular disulfide form) has no apparent cytotoxicity. Before conducting the EV uptake study, the influence of DTTox effluent on cell viability was evaluated. DTT (50 mM) was oxidated into nontoxic DTTox by running through an ES-EV Click Chip without performing EV capture. Subsequently, DTTox effluent was added into the cell culture medium of A673 cells and incubated at 37° C. for 24 h. As a negative control group, DPBS solution was added into the cell culture medium of A673 cells. Afterward, the Cell Counting Kit-8 (CCK-8) assay was used to test cell viability. As shown in FIG. 14B, DTTox effluent has a negligible effect on cell viability within 24 h.

To visualize the EV uptake process, PKH26-labeled ES-5838 EVs were purified by ES-EV Click Chips and co-cultured with A673 cells at 37° C. for 1, 2, and 4 h, respectively. A673 cells alone served as the negative controls (0 h). In parallel, the PKH26 negative control samples (without ES-5838 EVs) were also purified by ES-EV Click Chips and co-cultured with A673 cells. For static fluorescence imaging, A673 cells were washed with DPBS three times, fixed with 4% paraformaldehyde (PFA), stained with 4′,6-diamidino-2-phenylindole (DAPI), and imaged using a 40× objective lens on a Nikon Eclipse Ti fluorescence microscope under bright field, lasers 405 nm (DAPI) and 561 nm (PKH26). As shown in FIG. 14C, after co-culturing for 1 h, red fluorescent spots had bound to the surface of A673 cells and appeared inside the cells, indicating that the recovered PKH26-labeled ES-5838 EVs were taken up and internalized by A673 cells. With the extension of co-culturing time to 4 h, accumulating red fluorescent spots were observed inside A673 cells. In contrast, there was no red fluorescent signal in A673 cells of the corresponding negative control groups at 1, 2, and 4 h. The number of red fluorescent spots that were internalized by A673 cells after co-culturing were calculated for 1, 2, and 4 h. As summarized in FIG. 14D, the medians of spots per A673 cell with internalization were 3, 8, and 16 spots for 1, 2, and 4 h groups, respectively. The linear fitting curve (y=4.29x, R²=0.998) indicated an increasing trend of EV uptake over time. The dynamic process of EV uptake and internalization by live A673 cells was also photographed once every 15 min for 90 min using a 40× objective lens on the Nikon Eclipse Ti fluorescence microscope under bright field and laser 561 nm.

Furthermore, it has been recognized that EVs are able to transfer their RNA cargoes to recipient cells both in vitro and in vivo.^{[8]} Because the male ES-5838 cell-derived EVs harbor unique EWS-ERG rearrangement and sex-determining region of the Y-chromosome (SRY) transcripts, which are not present in female A673 cells, the EWS-ERG rearrangement and SRY expression could be used as specific molecular markers for quantification of ES-5838 EVs that were internalized by A673 cells. Therefore, after co-culturing with ES-5838 EVs for 1, 2, and 4 h, A673 cells in wells were washed with DPBS three times, treated with 0.25% trypsin-EDTA at 37° C. for 1 min and washed thoroughly with the citric acid buffer to remove the unbound EVs and cell surface-bound EVs. After centrifugation at 300 g for 10 min, A673 cell pellets were lysed by 700 μL of QIAzol lysis reagent and purified with miRNeasy Mini Kits (Qiagen). The purified RNA was subjected to RT-ddPCR quantification. Both EWS-ERG rearrangement and SRY transcript were detectable in A673 cells with ES-5838 EV uptake. As summarized in FIGS. 14E and 14F, the average copy numbers of EWS-ERG rearrangement and SRY transcript were (35 copies, 25 copies), (74 copies, 49 copies), (145 copies, 95 copies) for 1, 2, and 4 h groups, respectively. The linear fitting curves showed that the accumulation of EWS-ERG rearrangement (y=36.35x, R²=0.999) and SRY transcript (y=23.88x, R²=0.999) in the receipt cells were in a time-dependent manner. Altogether, these results demonstrate that the ES EVs recovered from ES-EV Click Chips exhibit well-preserved viability and can successfully transfer their RNA cargo contents to recipient cells.

FIGS. 14A-14F are schematics, graphs, and microscopic images showing results of downstream functional studies using the ES EVs purified by ES-EV Click Chips according to an embodiment of the invention. FIG. 14A is a schematic illustrating the functional study by co-culturing the purified ES-5838 EVs and A673 cells, resulting in the ES-5838 EV uptake and mRNA cargo transfer into A673 cells. FIG. 14B is a graph showing the effect of DTTox effluent on cell viability (%), which was calculated as the ratio of the OD₄₅₀ value of the DTTox effluent-added group (deducting the blank OD₄₅₀ value) to that of the negative control group (deducting the blank OD₄₅₀ value). FIG. 14C shows representative fluorescence micrograph images of A673 cells co-cultured with the purified PKH26-labeled ES-5838 EVs for 0, 1, 2, and 4 h. Nuclei of A673 cells were stained with DAPI. FIG. 14D shows box plots showing the number of fluorescence spots per A673 cell with internalized ES-5838 EVs after co-culturing for 0, 1, 2, and 4 h. The upper and lower box borders indicate the 25th and 75th percentiles, the horizontal line going through the box is the median, and the small solid square in the box (▪) is the mean. The diagonal line is the linear fitting curve made by the mean value. FIG. 14E is a graph showing the copy number of EWS-ERG rearrangement (specific molecular markers of ES-5838 EVs) detected within A673 cells after co-culturing with ES-5838 EVs for 0, 1, 2, and 4 h. FIG. 14F is a graph showing the copy number of SRY transcript (specific molecular markers of ES-5838 EVs) detected within A673 cells after co-culturing with ES-5838 EVs for 0, 1, 2, and 4 h.

### 3. Conclusion

A novel ES-EV purification system—ES-EV Click Chip—has been developed by coupling covalent chemistry-mediated EV capture/release within a nanostructure-embedded microchip. This device exploits anti-LINGO-1-specific recognition, sensitive click chemistry-mediated EV capture, and disulfide cleavage-driven EV release on a SiNWS-embedded microfluidic platform, realizing the highly efficient purification of ES EVs while maintaining their well-preserved integrity and biological activity. Fluorescence microscopy, TEM, SEM, and DLS characterization was adopted to demonstrate the EV capture and release features of ES-EV Click Chip. ES-EV Click Chip has several distinct advantages. (i) ES-EV Click Chips were optimized to have higher capture efficiency and lower antibody consumption compared with the previously reported Nano Villi Chips.^{[33]} This improvement is attributed to the rapid, chemoselective, and irreversible click chemistry-mediated capture mechanism, as well as the significantly increased number of click reaction sites between TCO moicties grafted on EVs and Tz moicties functionalized on Si nanowire arrays. (ii) Compared to other potential capture agents, such as anti-CD99 and anti-CD63, the use of anti-LINGO-1 in ES-EV Click Chips significantly improves the efficiency and specificity of ES-EV enrichment. (iii) Furthermore, the mild reagent DTT-mediated disulfide bond cleavage enables the subsequent release of ES EVs with high efficiency. Compared with other EV-capture and release strategies on nanostructured substrates (e.g., the immune-affinity EV capture/proteinase K and temperature-responsive dual EV release strategy^{[32]} and the non-specific exosome trapping/porous silicon nanowire dissolving strategy^{[63]}), ES-EV Click Chips could purify ES EVs under milder conditions with high specificity and isolation efficiency, enhanced reproducibility, reduced cost and time consumptions, as well as recovering tumor-derived EVs with well-preserved integrity for downstream functional studies. It was demonstrated that ES-EV Click Chip could purify ES EVs without any size bias and recover them with well-preserved viability and RNA cargo contents. The recovered ES EVs can be rapidly internalized and shuttle their RNA cargoes to recipient cells, which can be leveraged to explore their physiologic and potential pathologic roles in intercellular communication.

### 4. Experimental Section

Fabrication of ES-EV Click Chip Devices: ES-EV Click Chip device consists of (i) a Tz-grafted SiNWS and (ii) a PDMS-based chaotic mixer. Firstly, SiNWS with densely packed Si nanowires (diameters=100-200 nm, spacings=200-400 nm, lengths of 3-5 or 7-10 μm) were prepared by a combination of photolithographic patterning and AgNP-templated wet etching^{[54]} according to the following procedures: (i) (100) p-type Si wafers (Silicon Quest International) were spin-coated with a thin-film photoresist (AZ 5214, AZ Electronic Materials USA Corp.) and exposed to ultraviolet light; (ii) the wafers were immersed into etching solution with hydrofluoric acid (4.6 M, Sigma-Aldrich) and silver nitrate (0.2 M, Sigma-Aldrich); and (iii) the wafers were treated with boiling aqua regia [i.e., hydrochloric acid/nitric acid, 3:1 (v/v), Sigma Aldrich] to remove the silver film. The resultant SiNWS were incubated with a piranha solution [sulfuric acid/hydrogen peroxide, 2:1 (v/v), Sigma-Aldrich]. Next, Tz moieties with disulfide linkers were functionalized onto the SiNWS via a 3-step chemical modification^{[55]} process: (i) exposing the SiNWS to silane vapor of MPS (95%, 200 μL, Sigma-Aldrich) in a scaled vacuum desiccator for 45 min; (ii) incubating the SiNW with 200 μL of dimethyl sulfoxide (DMSO) solution containing OPSS-PEG-NH₂ (3.8 mM, Nanocs Inc.) for 2 h at room temperature; and (iii) further incubating the SiNW with 200 μL of PBS solution containing Tz-sulfo-NHS ester (3.8 mM; Click Chemistry Tools) for 1 h at room temperature. Thus, Tz-grafted SiNWS were produced and ready to use.

Secondly, PDMS-based microfluidic chaotic mixers^{[45]} were prepared by ICP-RIE.^{[56]} Briefly, (i) a master wafer was photolithographically prepared by spin-coating a layer of negative photoresist (MicroChem Corp.) with a thickness of 75 μm onto a silicon wafer; (ii) after exposure to UV light with a photomask containing a 2.0-mm-width serpentine rectangular microfluidic channel, the second layer of negative photoresist was spin-coated with a thickness of 40 μm; (iii) using a Mask Aligner (Karl Suss America Inc.), the second photomask containing herringbone ridges was aligned between the former pattern and the one to be imprinted; (iv) the Si master was exposed to trimethylchlorosilane (99%, Sigma-Aldrich) vapor for 1 min and transferred to a petri dish; (v) for replica molding, well-mixed PDMS precursor (RTV 615 A and B in a 10:1 ratio; GE Silicones) was filled into the petri dish, degassed, and incubated in an oven at 80° C.to make a 5-mm-thick chip; and (vi) the produced PDMS-based chaotic mixer was peeled off and punched with two through-holes at the ends of the serpentine rectangular microfluidic channel for insertion of tubing. Finally, the above Tz-grafted SiNWS and PDMS-based chaotic mixer were combined in a custom-designed chip holder to give an ES-EV Click chip device. Then, ES-EV Click chip device was placed in an automated digital fluidic handler to control the loading and flow of reagents and EV samples.

Preparation of TCO-Antibody Conjugates: The TCO-anti-LINGO-1 conjugate was produced by incubating TCO-PEG+-NHS ester (4 μM, Click Chemistry Tools) with polyclonal goat IgG human LINGO-1 antibody (1 μM, R&D Systems Inc.) in PBS solution (pH 7.4) at room temperature for 30 min. TCO-anti-CD99 and TCO-anti-CD63 conjugates were prepared accordingly by incubating TCO-PEG4-NHS ester (4 μM, Click Chemistry Tools) with polyclonal goat IgG human CD99 antibody (1 μM, R&D Systems Inc.) and Monoclonal Mouse IgG₁ human CD63 antibody (1 μM, R&D Systems Inc.), respectively. The resultant TCO-antibody conjugates (1 μM) in PBS solution were stored at −20° C. until use.

Culture of ES Cell Lines: ES cell lines, i.e., A673 cells (female origin, harboring EWS-FLI1 type 1 rearrangement) and SK-ES-1 cells (male origin, harboring EWS-FLI1 type 2 rearrangement) were obtained from the American Type Culture Collection (ATCC) and regularly tested negative for mycoplasma contamination. ES-5838 cells (male origin, harboring EWS-ERG rearrangement) were provided by Dr. James S. Tomlinson's Lab (UCLA). These cells were grown in 18 Nunc EasYDish dishes (150 mm, Thermo Fisher Scientific) with Dulbecco's Modified Eagle's Medium (DMEM, Thermo Fisher Scientific), fetal bovine serum (FBS, 10% (v/v), Thermo Fisher Scientific), GlutaMAX-I (1% (v/v), Thermo Fisher Scientific), and penicillin-streptomycin (100 U mL⁻¹, Thermo Fisher Scientific) in a humidified incubator with 5% CO₂ at 37° C. for three days.

Immunofluorescence Characterization of LINGO-1 and CD99 Expression on Cells: To demonstrate the specificity of LINGO-1 expression on ES cells, WBCs were isolated from the peripheral venous blood sample of a healthy donor with approval from UCLA Institutional Review Board (IRB, #00000173) and served as the control group of ES cells. A673 cells, ES-5838 cells, and WBCs on glass coverslips were detected with the following immunocytochemistry (ICC) protocol. First, cells were fixed with 4% PFA fixative solution (Electron Microscopy Sciences) for 20 min and subsequently incubated with 0.1% Triton X-100 for 10 min at room temperature. Next, these cells were incubated overnight at 4° C. with the primary antibody, i.e., polyclonal goat IgG human LINGO-1 antibody [1:100 (v/v)] or polyclonal goat IgG human CD99 antibody [1:40 (v/v)], in 200 μL of PBS containing 2% donkey serum (Jackson ImmunoResearch). After washing with PBS, these cells were incubated with the secondary antibody, i.e., donkey anti-goat IgG (H+L) [Alexa Fluor 647, 1:500 (v/v); Invitrogen] in 200 μL of PBS containing 2% donkey serum at room temperature for 1 h. After washing with PBS, these cells were treated with DAPI solution [1:1000 (v/v), Invitrogen]. Thereafter, these cells were imaged using a 40× objective lens on a Nikon Eclipse 90i fluorescence microscope.

Isolation and Preparation of ES-EV Samples: ES cells were cultured in serum-free medium for 24 h. A total of 234 mL of medium was collected in six Falcon 50 ml Conical Centrifuge Tubes (Thermo Fisher Scientific) and centrifuged at 4° C. and 300 g for 10 min to remove cells and cellular debris. The supernatant was centrifuged at 4° C. and 4,600 g for 30 min to eliminate large particles. Thereafter, the supernatant was transferred to six Ultra-Clear Tubes (38.5 mL, Beckman Coulter, Inc., USA) and centrifuged at 4° C. and 100,000 g for 2 h using Optima L-100 XP Ultracentrifuge (Beckman Coulter, Inc. USA). For making model EV samples, the resultant EV pellet was resuspended in 2 mL of serum-free medium and divided into 20 equal parts (each 100 μL). For making artificial EV plasma samples, the EV pellet was resuspended in 2 mL of blood plasma collected from a female healthy donor with approval from the UCLA Institutional Review Board (IRB, #00000173), and divided into 20 aliquots (each 100 μL). These ES-EV samples were stored at −80° C. for future use. EV Labeling with PKH26 Red-Fluorescent Dye: ES EVs were labeled with PKH26 red fluorescent cell linker kit (Sigma-Aldrich) according to the instructions with some modifications.^{[64]} Briefly, EV pellets were resuspended in 500 μL of Diluent C. Separately, 500 μL Diluent C was mixed with 2 μL of PKH26 red-fluorescent dye (1 mM) to prepare a 2× dye (4 μM) solution. After mixing the EV and PKH26 solution for 5 min at 4° C., 1 mL of 1% bovine serum albumin (BSA, Sigma-Aldrich) was added to bind excess dye. Then, the PKH26-labeled EVs were washed with PBS through ultracentrifugation at 4° C. and 100,000 g for 2 h to remove the free PKH26 dye. The pellet was resuspended in PBS and divided into several replicates. Meanwhile, as a negative control, PKH26 dye alone (without ES EVs) was also washed with PBS by ultracentrifugation and diluted in PBS to make the PKH26 negative control sample.

ES-EV Capture and Release by ES-EV Click Chips: Prior to capture, the ES-EV sample (100 μL) was pre-incubated with the TCO-LINGO-1 conjugate for 20 min at room temperature. Meanwhile, 200 μL of PBS was injected into ES-EV Click Chip at a flow rate of 1 mL h⁻¹ to test leaks. The resultant TCO-grafted EV sample was then introduced into ES-EV Click Chip at an optimal flow rate of 0.2 mL h⁻¹ and captured on the Tz-grafted SiNWS via the click chemistry-mediated EV capture. Afterward, to release the EVs captured on chips, a DPBS solution (50-100 μL) containing DTT (50 mM) was injected into ES-EV Click Chip at an optimal flow rate of 0.2 mL h⁻¹. The released EVs were collected into a 1.5-mL ribonuclease (RNase)-free Eppendorf tube.

TEM Characterization: The ES EVs in solution or Si nanowires mechanically detached from the SiNWS after EV capture/release were fixed in 4% PFA for 30 min at room temperature. Next, 5 μL of samples were placed onto formvar and carbon-coated copper grids (200-mesh) and incubated for 5 min. After blotting the excess samples with filter paper, grids were negatively stained with 2% uranyl acetate for 10 min. After rinsing with deionized water three times, samples were dried and imaged by JEM1200-EX (JEOL USA Inc.) at 80 kV.

For immunogold-TEM, 5 μL of samples were placed onto formvar and carbon-coated nickel grids (200-mesh) and incubated for 5 min. After wiping off the excess samples, grids were blocked in a blocking solution containing 0.4% BSA for 30 min and rinsed with deionized water three times. Then samples were incubated with monoclonal mouse IgG₁ human LINGO-1 antibody [clone #332237, 1:1000 (v/v), R&D Systems Inc.] or monoclonal mouse anti-CD63 (1:500 (v/v), Abcam] for 1 h. Meanwhile, samples were incubated with the blocking solution as negative controls. After rinsing with deionized water three times, the samples were incubated with goat anti-mouse IgG H&L 10-nm gold [1:40 (v/v), Abcam] for 1 h. Thereafter, grids were rinsed and negatively stained with 2% uranyl acetate, followed by drying and TEM imaging.

SEM Characterization: To characterize the distribution of EVs on Si nanowire arrays after capture/release, SiNWS were cut to expose the cross-sections of Si nanowire arrays and incubated with 4% PFA for 1 h at room temperature. Next, the substrates were dehydrated by sequentially immersing in 30%, 50%, 75%, 85%, 95%, and 100% ethanol solutions for 10 min per solution. After drying, the substrates were sputter-coated with gold and imaged under a ZEISS Supra 40VP SEM at an accelerating voltage of 10 keV.

DLS Characterization: The size distributions of EVs before capture and after release were measured using Malvern Zetasizer Nano ZS. EV samples were diluted 1:10 or 1:20 in the cuvette and analyzed by Malvern Zetasizer Nano ZS to give the size distribution.

Extraction and Quantification of RNA from ES EVs: For EVs captured on ES-EV Click Chip, RNA was extracted by introducing 700 μL of QIAzol lysis reagent at a flow rate of 0.5 mL h⁻¹ for 200 μL and then 60 mL h⁻¹ for the leftover 500 μL. The outflow was collected in a 1.5-mL ribonuclease (RNase)-free Eppendorf tube. For EVs before capture and after release in solution, 700 μL of QIAzol lysis reagent was added to lyse EVs in 1.5-mL ribonuclease (RNase)-free Eppendorf tubes. The extracted EV-derived RNA was purified with miRNeasy Micro Kits (Qiagen), according to the manufacturer's protocol. During the RNA purification process, DNase I (RNase-free, Thermo Fisher Scientific) was used to digest DNA for 15 min at room temperature. Finally, RNA was dissolved in DNase/RNase-free water and centrifuged off the RNeasy MinElute Spin Columns into 1.5-mL ribonuclease (RNase)-free collection tubes. The RNA was quantified with Qubit 3.0 Fluorometer (Thermo Fisher Scientific, USA) and Qubit RNA HS Assay according to the manufacturer's instructions.

RT-ddPCR Detection: RNA was reverse-transcribed to cDNA with a Maxima H Minus Reverse Transcriptase Kit (Thermo Fisher Scientific, USA) according to the manufacturer's protocol. The reverse transcription reaction was performed at 55° C. for 30 min and 85° C. for 5 min. Thereafter, cDNA was detected with ddPCR Supermix for Probes (No dUTP, Bio-Rad). EWS rearrangements were detected using self-designed primers and probes. SRY transcript was detected using a commercial primer/probe kit (Catalog #4331182; Assay ID: Hs00976796_s1, Thermo Fisher Scientific). Droplets containing ddPCR reaction were transferred into a 96-well plate and sealed. ddPCR reaction was performed at 96° C. for 10 min, followed by 40 cycles (94° C. for 30 s and 60° C. for 60 s) and 98° C. for 10 min. The DNA amplicons contained in droplets were detected by a QX200 Droplet Reader in combination with a QuantaSoft™ software package.

Comparison with Immunomagnetic Beads, Ultracentrifugation, and ExoQuick ULTRA EV Isolation Kit: For immunomagnetic bead separation, Tz-grafted magnetic beads were prepared by incubating 2.8 μm Dynabeads™ M-270 Amine (2×10⁸ beads, 100 μL, Thermo Fisher Scientific) with Tz-sulfo-NHS ester (0.32 mg, Click Chemistry Tools, USA) in PBS buffer for 1 h at room temperature. Each artificial A673 EV plasma sample was pre-incubated with the TCO-LINGO-1 conjugate (1 pmol) for 20 min and incubated with Tz-grafted magnetic beads (2×10⁷ beads) at room temperature for 30 min to isolate A673 EVs. For ultracentrifugation, each artificial A673 EV plasma sample was centrifuged at 100,000 g for 2 h using Optima L-100 XP Ultracentrifuge. For the commercially used EV isolation assay, each artificial A673 EV plasma sample was isolated and purified using the ExoQuick ULTRA EV Isolation Kit (System Biosciences) according to the manufacturer's protocol. For all the methods, RNA was extracted from the isolated EVs and quantified using Qubit 3.0 Fluorometer (Thermo Fisher Scientific), followed by quantification of EWS-FLI1 type 1 rearrangement using RT-ddPCR detection. Healthy-donor plasma samples without A673 EVs were processed in parallel to give the systems' RNA background.

CCK-8 Cell Viability Assay: A673 cells (5×10³ cell/well) were evenly plated into a sterile 96-well cell culture plate with the cell culture medium (250 μL per well) and pre-incubated in a humidified incubator with 5% CO₂ at 37° C. for 24 h. For the DTTox effluent-added group, 50 μL of DTTox effluent was added into each well and incubated with A673 cells for 24 h. For the negative control, 50 μL of DPBS solution was incubated with A673 cells for 24 h. Thereafter, CCK-8 (Sigma-Aldrich) assay was used to test the effect of DTTox effluent on cell viability. The cell culture medium of A673 cells in each well was replaced with 10 μL of CCK-8 solution and 100 μL of serum-free medium. Meanwhile, a blank well without A673 cells was also added with 10 μL of CCK-8 solution and 100 μL of serum-free medium to serve as the blank of CCK-8 assay. After incubating for 4 h, the solution of each well was transferred to a Costar 96 Flat Transparent plate and placed into the Tecan Infinite 200 PRO. The optical density (OD, absorbance) at 450 nm was measured with an i-control Microplate Reader. The cell viability (%) was calculated as the ratio of the OD₄₅₀ value of the DTTox effluent-added group (deducting the blank OD₄₅₀ value) to that of the negative control group (deducting the blank OD₄₅₀ value).

Downstream Functional Studies: A673 cells (5×10³ cell/well) were evenly plated into a sterile 96-well cell culture plate with the cell culture medium (250 μL per well) and pre-incubated in a humidified incubator with 5% CO₂ at 37° C. for 24-48 h. Then the cell culture medium was replaced with serum-free medium (250 μL per well) for EV uptake study. Before the addition of ES-5838 EVs, the wells with A673 cells alone served as the negative controls (0 h). After being released from ES-EV Click Chip, PKH26-labeled ES-5838 EVs (50 μL) were added to the wells and co-cultured with A673 cells at 37° C. for 1, 2, and 4 h, respectively. In parallel, the PKH26 negative control sample was also purified by ES-EV Click Chips and co-cultured with A673 cells at 37° C. for 1, 2, and 4 h, respectively.

Fluorescence Imaging of Uptake Process of ES EVs into Recipient Cells: For static fluorescence imaging, A673 cells were washed with DPBS three times, fixed with 4% PFA for 10 min, stained with DAPI [1:1000 (v/v)] for 10 min, and imaged using a 40× objective lens on a Nikon Eclipse Ti fluorescence microscope under bright field, lasers 405 nm (DAPI) and 561 nm (PKH26). For dynamic monitoring of the ES-5838 EV uptake and internalization process by live A673 cells, the 96-well cell culture plate was placed on the 3D automatic objective table and photographed once every 15 min for 90 min using a 40× objective lens on the Nikon Eclipse Ti fluorescence microscope under bright field and laser 561 nm (PKH26).

Detection of Gene Transcripts in Recipient Cells After EV Uptake. For detecting EWS-ERG rearrangement and SRY transcript of ES-5838 EVs internalized by A673 cells after co-culturing, A673 cells were washed with DPBS three times and treated with 0.25% trypsin-EDTA (Thermo Fisher Scientific) at 37° C. for 1 min and washed thoroughly with the citric acid buffer to remove the unbound EVs and cell surface-bound EVs. A673 cells were centrifuged at 300 g for 10 min. The cell pellets were lysed by 700 μL of QIAzol lysis reagent and purified with miRNeasy Mini Kits (Qiagen) according to the manufacturer's protocol. The purified RNA was subjected to RT-ddPCR detection.

### References from Example 2 and Background

[1] G. Raposo, W. Stoorvogel, J. Cell Biol. 2013, 200, 373.
[2] G. van Niel, G. D'Angelo, G. Raposo, Nat. Rev. Mol. Cell Biol. 2018, 19, 213.
[3] S. L. N. Maas, X. O. Breakefield, A. M. Weaver, Trends Cell Biol. 2017, 27, 172.
[4] S. Maacha, A. A. Bhat, L. Jimenez, A. Raza, M. Haris, S. Uddin, J. C. Grivel, Mol. Cancer 2019, 18, 55.
[5] S. Das, K. M. Ansel, M. Bitzer, X. O. Breakefield, A. Charest, D. J. Galas, M. B. Gerstein, M. Gupta, A. Milosavljevic, M. T. McManus, T. Patel, R. L. Raffai, J. Rozowsky, M. E. Roth, J. A. Saugstad, K. Van Keuren-Jensen, A. M. Weaver, L. C. Laurent, Cell 2019, 177, 231.
[6] L. Huang, N. Gu, X.-E. Zhang, D.-B. Wang, Adv. Funct. Mater. 2019, 29, 1807189.
[7] Y. Hong, G.-H. Nam, E. Koh, S. Jeon, G. B. Kim, C. Jeong, D.-H. Kim, Y. Yang, I.-S. Kim, Adv. Funct. Mater. 2018, 28, 1703074.
[8] G. Raposo, P. D. Stahl, Nat. Rev. Mol. Cell Biol. 2019, 20, 509.
[9] Y. Zhang, X. Jin, J. Liang, Y. Guo, G. Sun, X. Zeng, H. Yin, Sci. Adv. 2019, 5, eaav1564.
[10] Y. Jin, J. S. Lee, S. Min, H.-J. Park, T. J. Kang, S.-W. Cho, Adv. Funct. Mater. 2016, 26, 5804.
[11] S. Raimondo, G. Giavaresi, A. Lorico, R. Alessandro, Int. J. Mol. Sci. 2019, 20, 1848.
[12] R. Rahbarghazi, N. Jabbari, N. A. Sani, R. Asghari, L. Salimi, S. A. Kalashani, M. Feghhi, T. Etemadi, E. Akbariazar, M. Mahmoudi, J. Rezaie, Cell Commun. Signal. 2019, 17, 73.
[13] J. Hu, Y. Sheng, K. J. Kwak, J. Shi, B. Yu, L. J. Lee, Nat. Commun. 2017, 8, 1683.
[14] J. Zhao, C. Liu, Y. Li, Y. Ma, J. Deng, L. Li, J. Sun, J. Am. Chem. Soc. 2020, 142, 4996.
[15] M. Tkach, C. Thery, Cell 2016, 164, 1226.
[16] M. Mathieu, L. Martin-Jaular, G. Lavieu, C. Théry, Nat. Cell Biol. 2019, 21, 9.
[17] B. J. Tauro, D. W. Greening, R. A. Mathias, H. Ji, S. Mathivanan, A. M. Scott, R. J. Simpson, Methods 2012, 56, 293.
[18] K. Rekker, M. Saare, A. M. Roost, A.-L. Kubo, N. Zarovni, A. Chiesi, A. Salumets, M. Peters, Clin. Biochem. 2014, 47, 135.
[19] M. L. Heinemann, M. Ilmer, L. P. Silva, D. H. Hawke, A. Recio, M. A. Vorontsova, E. Alt, J. Vykoukal, J. Chromatogr. A 2014, 1371, 125.
[20] S. S. Kanwar, C. J. Dunlay, D. M. Simeone, S. Nagrath, Lab Chip 2014, 14, 1891.
[21] B. H. Wunsch, J. T. Smith, S. M. Gifford, C. Wang, M. Brink, R. L. Bruce, R. H. Austin, G. Stolovitzky, Y. Astier, Nat. Nanotechnol. 2016, 11, 936.
[22] C. Liu, J. Guo, F. Tian, N. Yang, F. Yan, Y. Ding, J. Wei, G. Hu, G. Nie, J. Sun, ACS Nano 2017, 11, 6968.
[23] Y. Wan, G. Cheng, X. Liu, S.-J. Hao, M. Nisic, C.-D. Zhu, Y.-Q. Xia, W.-Q. Li, Z.-G. Wang, W.-L. Zhang, S. J. Rice, A. Sebastian, I. Albert, C. P. Belani, S.-Y. Zheng, Nat. Biomed. Eng. 2017, 1, 0058.
[24] W. Wang, J. Luo, S. Wang, Adv. Healthc. Mater. 2018, 7, 1800484.
[25] W. Li, H. Wang, Z. Zhao, H. Gao, C. Liu, L. Zhu, C. Wang, Y. Yang, Adv. Mater. 2019, 31, 1805344.
[26] C. Liu, J. Zhao, F. Tian, J. Chang, W. Zhang, J. Sun, J. Am. Chem. Soc. 2019, 141, 3817.
[27] C. Liu, J. Zhao, F. Tian, L. Cai, W. Zhang, Q. Feng, J. Chang, F. Wan, Y. Yang, B. Dai, Y. Cong, B. Ding, J. Sun, W. Tan, Nat. Biomed. Eng. 2019, 3, 183.
[28] W. Li, B. Shao, C. Liu, H. Wang, W. Zheng, W. Kong, X. Liu, G. Xu, C. Wang, H. Li, L. Zhu, Y. Yang, Small Methods 2018, 2, 1800122.
[29] R. Jauregui, S. Srinivasan, L. N. Vojtech, H. S. Gammill, D. T. Chiu, F. Hladik, P. S. Stayton, J. J. Lai, ACS Appl. Mater. Interfaces 2018, 10, 33847.
[30] G. Qiu, A. Thakur, C. Xu, S.-P. Ng, Y. Lee, C.-M. L. Wu, Adv. Funct. Mater. 2019, 29, 1806761.
[31] S. A. Melo, L. B. Luecke, C. Kahlert, A. F. Fernandez, S. T. Gammon, J. Kaye, V. S. LeBleu, E. A. Mittendorf, J. Weitz, N. Rahbari, C. Reissfelder, C. Pilarsky, M. F. Fraga, D. Piwnica-Worms, R. Kalluri, Nature 2015, 523, 177.
[32] E. Reátegui, K. E. van der Vos, C. P. Lai, M. Zeinali, N. A. Atai, B. Aldikacti, F. P. Floyd, A. H. Khankhel, V. Thapar, F. H. Hochberg, L. V. Sequist, B. V. Nahed, B. S. Carter, M. Toner, L. Balaj, D. T. Ting, X. O. Breakefield, S. L. Stott, Nat. Commun. 2018, 9, 175.
[33] J. Dong, R. Y. Zhang, N. Sun, M. Smalley, Z. Wu, A. Zhou, S. J. Chou, Y. J. Jan, P. Yang, L. Bao, D. Qi, X. Tang, P. Tseng, Y. Hua, D. Xu, R. Kao, M. Meng, X. Zheng, Y. Liu, T. Vagner, X. Chai, D. Zhou, M. Li, S. H. Chiou, G. Zheng, D. Di Vizio, V. G. Agopian, E. Posadas, S. J. Jonas, S. P. Ju, P. S. Weiss, M. Zhao, H. R. Tseng, Y. Zhu, ACS Appl. Mater. Interfaces 2019, 11, 13973.
[34] T. G. P. Grünewald, F. Cidre-Aranaz, D. Surdez, E. M. Tomazou, E. de Álava, H. Kovar, P. H. Sorensen, O. Delattre, U. Dirksen, Nat. Rev. Dis. Primers 2018, 4, 5.
[35] A. Villasante, A. Marturano-Kruik, S. R. Ambati, Z. Liu, A. Godier-Furnemont, H. Parsa, B. W. Lee, M. A. Moore, G. Vunjak-Novakovic, Theranostics 2016, 6, 1119.
[36] A. De Feo, M. Sciandra, M. Ferracin, F. Felicetti, A. Astolfi, Y. Pignochino, P. Picci, A. Care, K. Scotlandi, Cell Death Dis. 2019, 10, 471.
[37] I. V. Miller, G. Raposo, U. Welsch, O. P. da Costa, U. Thiel, M. Lebar, M. Maurer, H. U. Bender, I. von Luettichau, G. H. S. Richter, S. Burdach, T. G. P. Grunewald, Biol. Cell 2013, 105, 289.
[38] M. Tsugita, N. Yamada, S. Noguchi, K. Yamada, H. Moritake, K. Shimizu, Y. Akao, T. Ohno, PLOS One 2013, 8, e77416.
[39] P. Zhang, J. Crow, D. Lella, X. Zhou, G. Samuel, A. K. Godwin, Y. Zeng, Lab Chip 2018, 18,3790.
[40] J. Town, H. Pais, S. Harrison, L. F. Stead, C. Bataille, W. Bunjobpol, J. Zhang, T. H. Rabbitts, Proc. Natl. Acad. Sci. U.S.A. 2016, 113, 3603.
[41] N. K. Devaraj, ACS Central Sci. 2018, 4, 952.
[42] D. Witt, Synthesis 2008, 2008, 2491.
[43] S. Wang, H. Wang, J. Jiao, K. J. Chen, G. E. Owens, K. Kamei, J. Sun, D. J. Sherman, C. P. Behrenbruch, H. Wu, H. R. Tseng, Angew. Chem. Int. Ed. 2009, 48, 8970.
[44] J. Dong, J.-F. Chen, M. Smalley, M. Zhao, Z. Ke, Y. Zhu, H.-R. Tseng, Adv. Mater. 2020, 32, 1903663.
[45] S. Wang, K. Liu, J. Liu, Z. T. Yu, X. Xu, L. Zhao, T. Lee, E. K. Lee, J. Reiss, Y. K. Lee, L. W. Chung, J. Huang, M. Rettig, D. Seligson, K. N. Duraiswamy, C. K. Shen, H. R. Tseng, Angew. Chem. Int. Ed. 2011, 50, 3084.
[46] M. L. Blackman, M. Royzen, J. M. Fox, J. Am. Chem. Soc. 2008, 130, 13518.
[47] B. L. Oliveira, Z. Guo, G. J. L. Bernardes, Chem. Soc. Rev. 2017, 46, 4895.
[48] M. R. Karver, R. Weissleder, S. A. Hilderbrand, Bioconjug. Chem. 2011, 22, 2263.
[49] E. A. Smith, M. J. Wanat, Y. Cheng, S. V. P. Barreira, A. G. Frutos, R. M. Corn, Langmuir 2001, 17, 2502.
[50] S. Toki, S. Wakai, M. Sekimizu, T. Mori, H. Ichikawa, A. Kawai, A. Yoshida, Histopathology 2018, 73, 645.
[51] P. Zhang, G. Samuel, J. Crow, A. K. Godwin, Y. Zeng, Transl. Res. 2018, 201, 136.
[52] S. Benini, G. Gamberi, S. Cocchi, J. Garbetta, L. Alberti, A. Righi, M. Gambarotti, P. Picci, S. Ferrari, Cancer Manag. Res. 2018, 10, 49.
[53] M. Tellez-Gabriel, H. K. Brown, R. Young, M. F. Heymann, D. Heymann, Front. Oncol. 2016, 6, 202.
[54] K. Q. Peng, Y. J. Yan, S. P. Gao, J. Zhu, Adv. Mater. 2002, 14, 1164.
[55] J. Dong, Y. J. Jan, J. Cheng, R. Y. Zhang, M. Meng, M. Smalley, P. J. Chen, X. Tang, P. Tseng, L. Bao, T. Y. Huang, D. Zhou, Y. Liu, X. Chai, H. Zhang, A. Zhou, V. G. Agopian, E. M. Posadas, J. J. Shyue, S. J. Jonas, P. S. Weiss, M. Li, G. Zheng, H. H. Yu, M. Zhao, H. R. Tseng, Y. Zhu, Sci. Adv. 2019, 5, eaav9186.
[56] W. Sheng, O. O. Ogunwobi, T. Chen, J. Zhang, T. J. George, C. Liu, Z. H. Fan, Lab Chip 2014, 14, 89.
[57] J. B. Haun, N. K. Devaraj, S. A. Hilderbrand, H. Lee, R. Weissleder, Nat. Nanotechnol. 2010, 5, 660.
[58] J. Kowal, G. Arras, M. Colombo, M. Jouve, J. P. Morath, B. Primdal-Bengtson, F. Dingli, D. Loew, M. Tkach, C. Théry, Proc. Natl. Acad. Sci. U.S.A. 2016, 113, E968.
[59] R. D. Groot, P. B. Warren, J. Chem. Phys. 1997, 107, 4423.
[60] H. Kalra, C. G. Adda, M. Liem, C. S. Ang, A. Mechler, R. J. Simpson, M. D. Hulett, S. Mathivanan, Proteomics 2013, 13, 3354.
[61] M. M. Halleck, H. Liu, J. North, J. L. Stevens, J. Biol. Chem. 1997, 272, 21760.
[62] S. Asmellash, J. L. Stevens, T. Ichimura, Toxicol. Sci. 2005, 88, 576.
[63] Z. Wang, H.-j. Wu, D. Fine, J. Schmulen, Y. Hu, B. Godin, J. X. J. Zhang, X. Liu, Lab Chip 2013, 13, 2879.
[64] C. A. Franzen, P. E. Simms, A. F. Van Huis, K. E. Foreman, P. C. Kuo, G. N. Gupta, BioMed Res. Int. 2014, 2014, 619829.

### Example 3

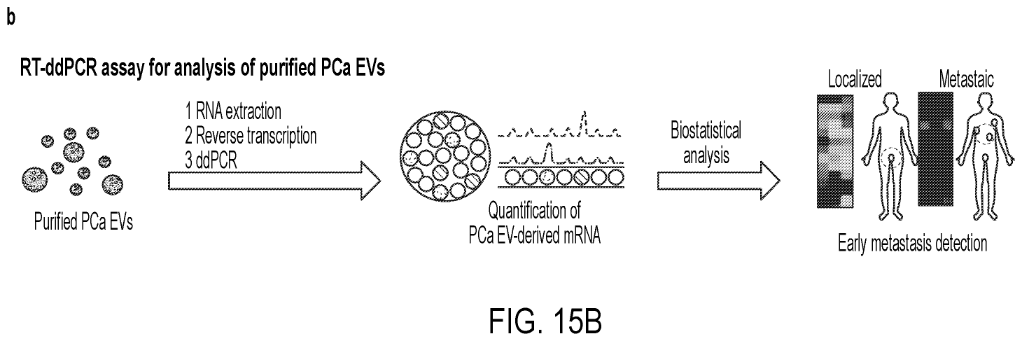

FIGS. 15A and 15B are schematics showing use of EV Click Chips for purification and molecular characterization of prostate cancer extracellular vesicles (PCa EVs). FIG. 15A, Schematic illustration of the device configuration and work mechanism of an EV Click Chip, which is composed of a patterned Si nanowire substrate (SiNWS) covalently functionalized with tetrazine (Tz), and an overlaid polydimethylsiloxane (PDMS) chaotic mixer. The covalent chemistry-mediated EV purification approach combines the click chemistry-mediated EV capture and disulfide cleavage-driven EV release in conjunction with the use of dual-antibody targeting 2 PCa-associated surface markers, i.e., EpCAM, and PSMA. A pair of highly reactive click chemistry motifs, i.e., Tz and trans-cyclooctene (TCO), are grafted onto SiNWS and EVs, respectively. When a plasma sample flows through the device, click chemistry reaction between Tz-grafted SiNWS and TCO-grafted PCa EVs results in the immobilization of the PCa EVs. Subsequently, the exposure to 1,4-dithiothreitol (DTT) leads to the cleavage of the embedded disulfide bonds to release the immobilized PCa EVs. FIG. 15B, The purified PCa EVs can then be subjected to reverse-transcription droplet digital PCR (RT-ddPCR) to obtain the signatures of PCa-specific genes, which can be used to distinguish localized PCa patients from at-risk metastatic patients.

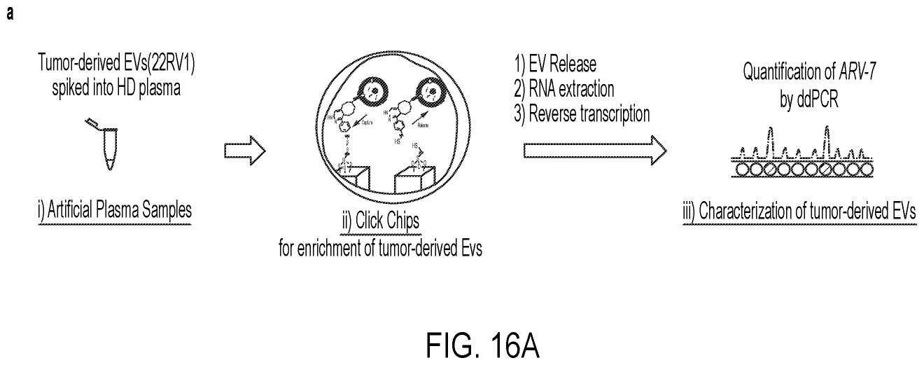

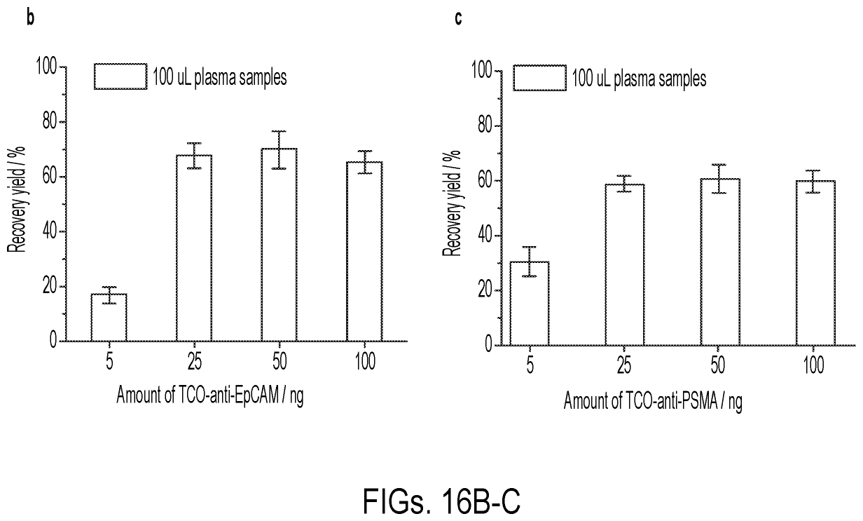

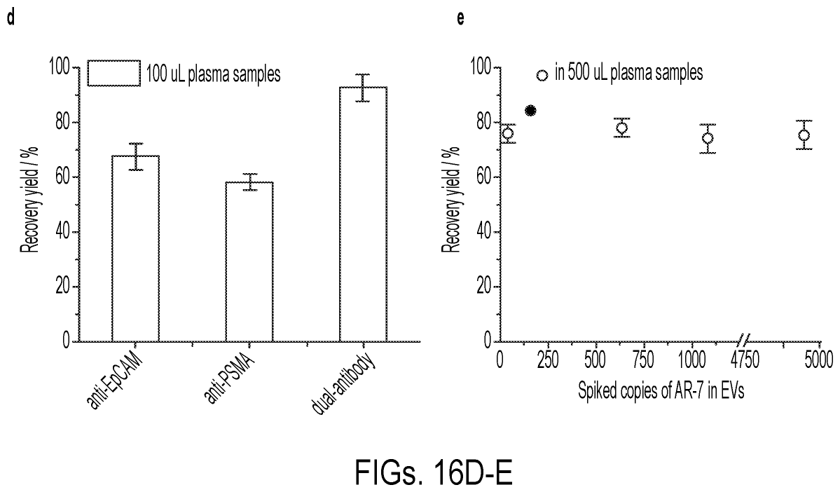

FIGS. 16A-16E are schematics and graphs showing optimization of EV Click Chips for capture of PCa-derived EVs using artificial plasma samples. FIG. 16A shows workflow for optimization of EV Click Chip using the artificial plasma samples, which were prepared by spiking 10-μL 22RV1 cell-derived EVs into 90-μL healthy-donor's plasma. After EV capturing and releasing, the recovered PCa-derived EVs were subjected to ddPCR, and the copy numbers of ARV-7 gene were calculated for the capturing efficiency. FIGS. 16B-16E, The recovery rates obtained in the presence of individual and combined antibody capture agents, i.e., (FIG. 16B) anti-EpCAM (n=3), (FIG. 16C) anti-PSMA (n=3), and (FIG. 16D) combination of the three capture agents (n=3). FIG. 16E, Dynamic ranges of EV recovery rates observed for EV Click Chips using artificial sample containing 0 to 5000 copies of ARV-7 transcripts.

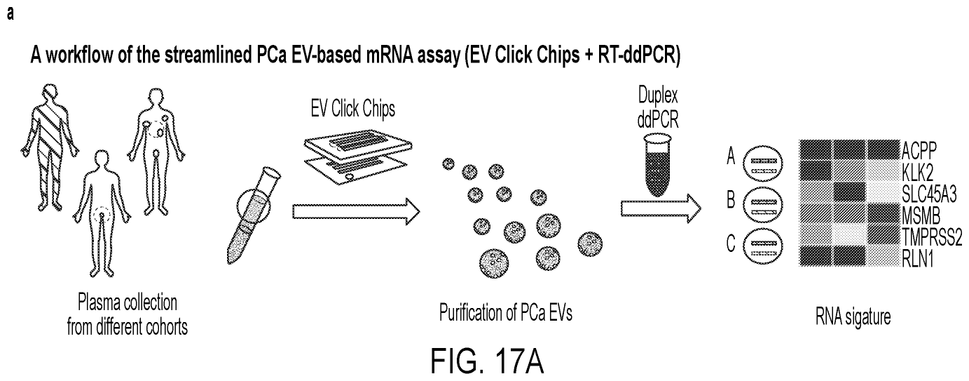

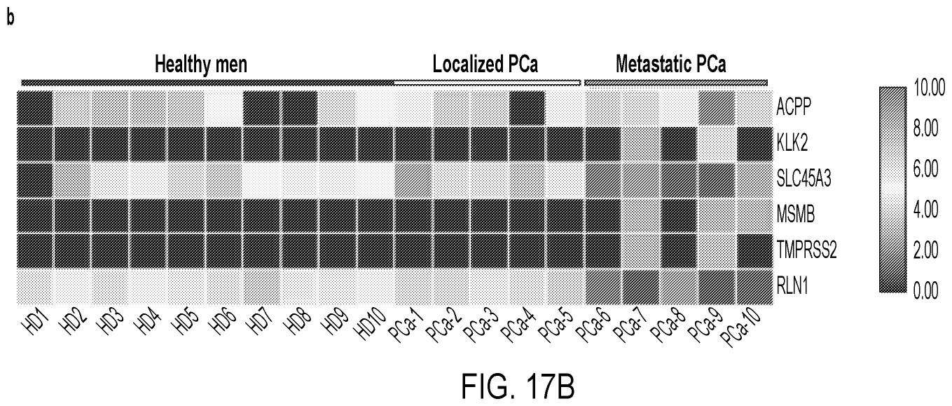

FIGS. 17A and 17B show results of a RT-ddPCR assay for quantification of 6 PCa-specific mRNA transcripts in PCa EVs purified by EV Click Chips. FIG. 17A is a general workflow developed for conducting PCa EV purification, followed by quantification of 6 PCa-specific mRNA transcripts in the purified PCa EVs. FIG. 17B are heat maps depicting relative signal intensities for each gene expression of the 6 PCa-specific genes across different cohorts. Primary copy numbers are log2-transformed and scaled to the highest value for each gene across all disease states.

### Example 4

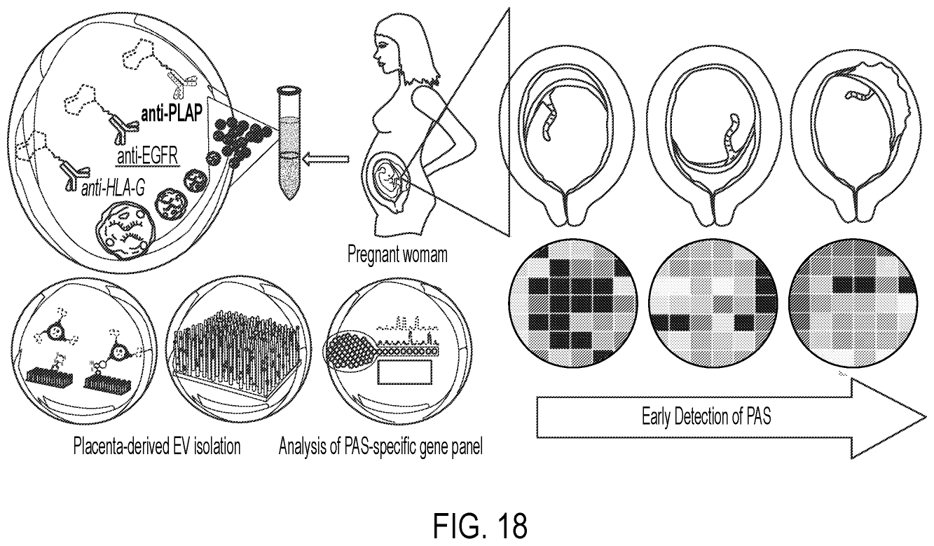

FIG. 18 is a schematic showing use of EV Click Chips for purification and molecular characterization of Placenta-derived EVs for placenta disease detection.

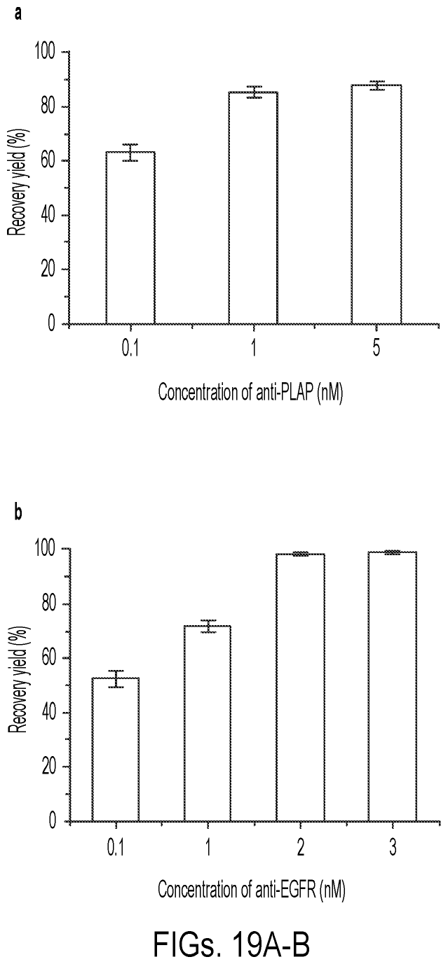

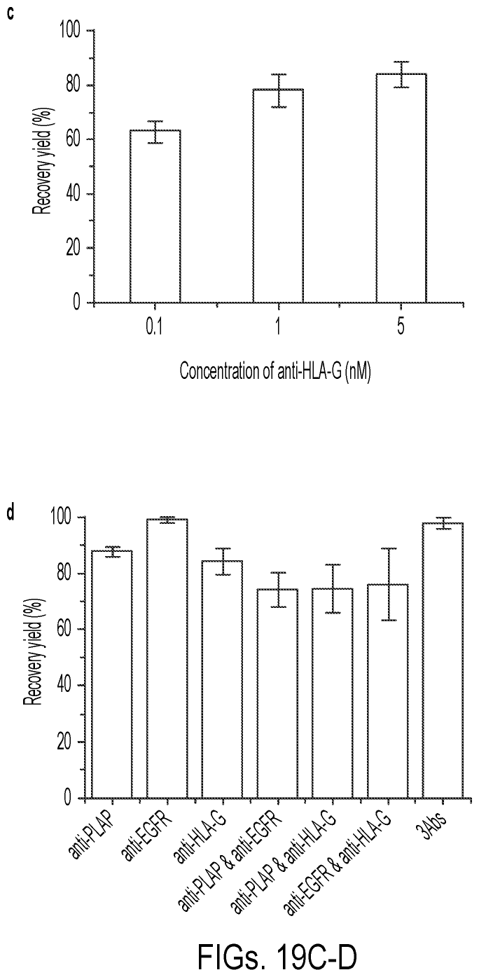

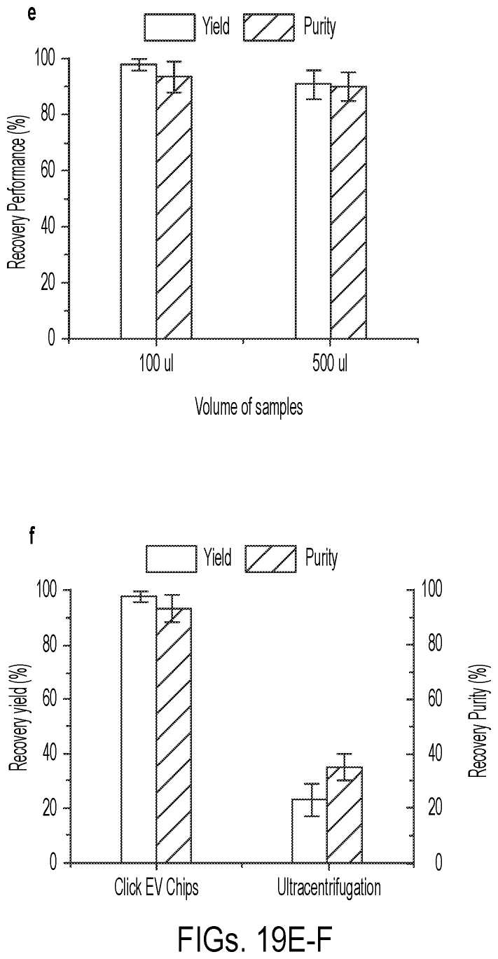

FIGS. 19A-19F are graphs showing optimization of EV Click Chips for capture of placenta-derived EVs using artificial plasma samples. Artificial plasma samples were prepared by spiking 10-μL JAR (a male cell line) cell-derived EVs into 90-μL healthy female donor's plasma. After EV capturing and releasing, the recovered placenta-derived EVs were subjected to ddPCR, and the copy numbers of SRY gene were calculated for the capturing efficiency. FIGS. 18A-18E, The recovery yields obtained in the presence of individual and combined antibody capture agents, i.e., (FIG. 19A) anti-PLAP (n=3), (FIG. 19B) anti-EGFR (n=3), (FIG. 19C) anti-HLA-G (n=3), and (FIG. 19D) combination of the three capture agents (n=3). FIG. 19E, The recovery yields and purities of EV Click Chips observed from different volume of plasma with the same amount of spiked JAR EVs, FIG. 19F, Comparison of recovery yields and purities observed for EV Click Chips and ultracentrifugation from artificial plasma samples (n=3).

### Example 5

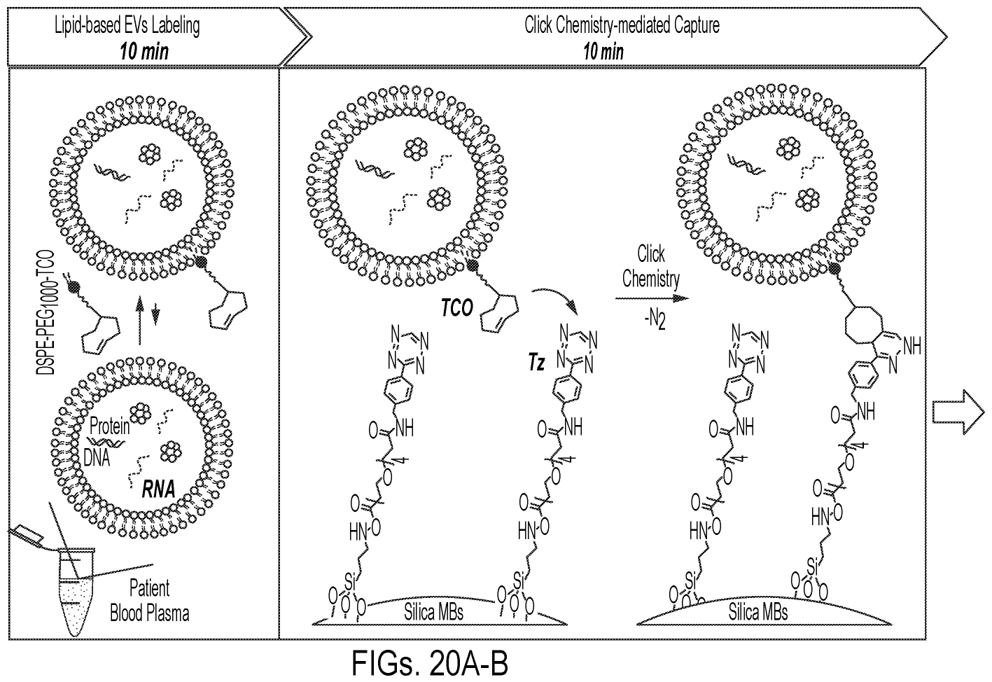

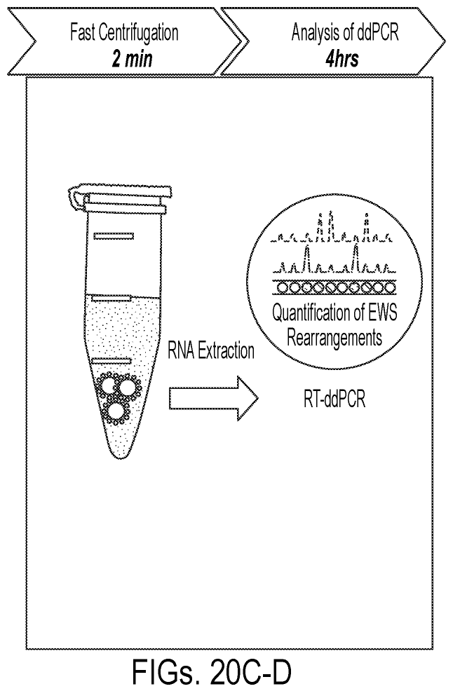

FIGS. 20A-20D are a schematic overview of rapid isolation and analysis system for EVs from patient blood plasma samples. FIG. 20A) Label: EVs in blood plasma are labeled by integration of a lipid molecule (DSPE-PEG1000-TCO). FIG. 20B) High-efficiency capture: the labeled EVs are captured onto the tetrazine modified silica microbeads (silica MBs) by click chemistry. FIG. 20C) Fast isolation: the captured EVs are enriched from blood plasma by centrifugation with silica MBs (2.0 g/cm³). FIG. 20D) The isolated EVs are then lysed to release EV-derived RNA, which is extracted for downstream analysis by reverse transcription Droplet Digital™ PCR (RT-ddPCR). This workflow was utilized to quantitatively detect gene alterations (i.e., EWS rearrangements) in Ewing sarcoma.

### Example 6

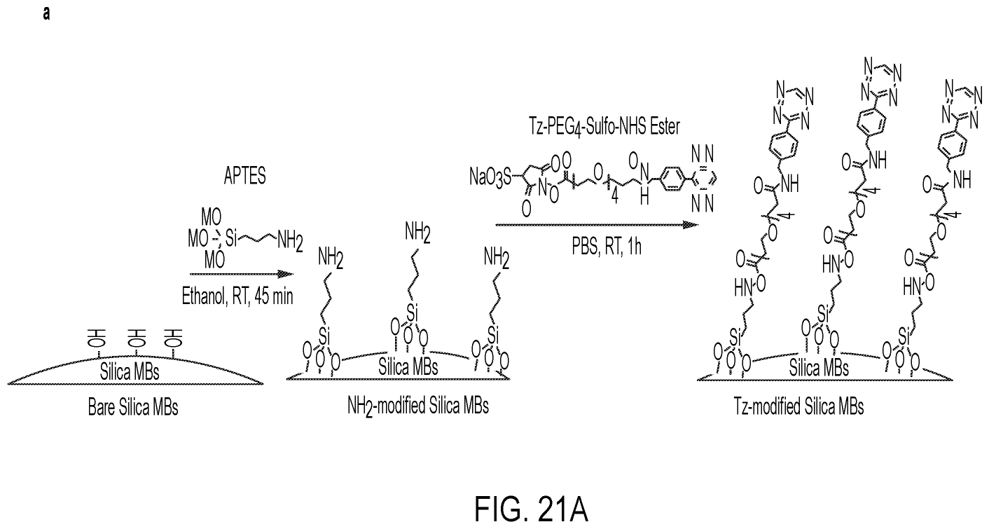

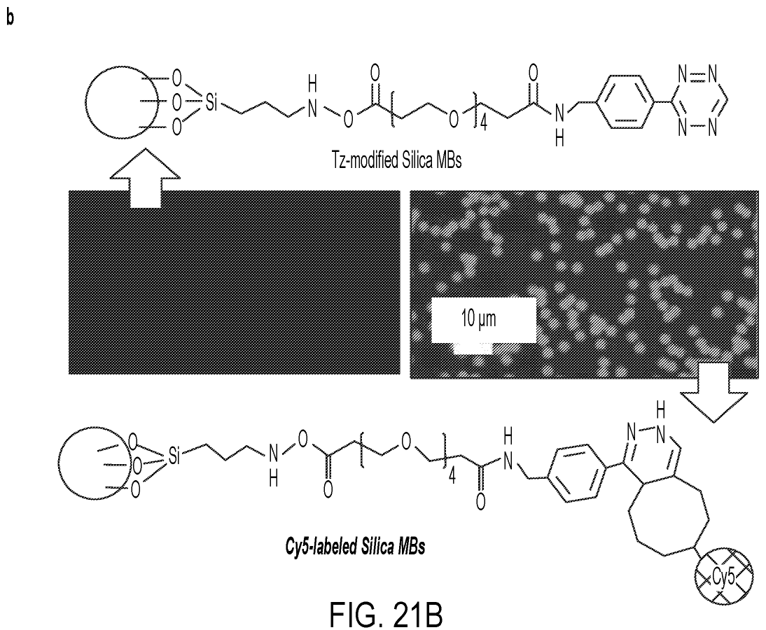

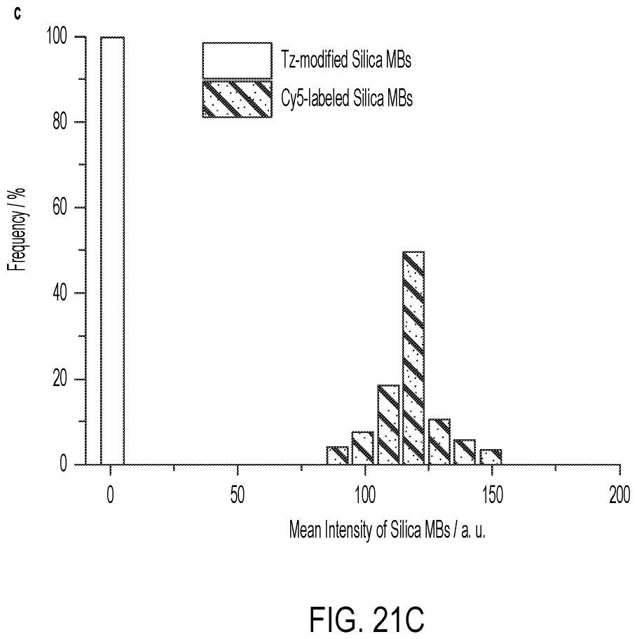

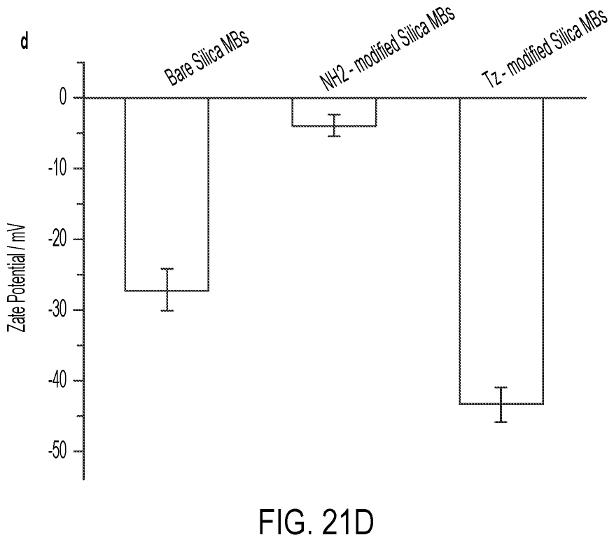

FIGS. 21A-21D are schematics and graphs showing surface modification and characterization of Tz-grafted silica microbeads (silica MBs). FIG. 21A is a schematic illustration of fabrication of Tz-grafted silica MBs. FIG. 21B are fluorescent images and FIG. 21C) histogram statistics of average fluorescence intensity for Tz-grafted silica MBs and Cy5-grafted silica MBs to validate the effectiveness of Tz motif grafted onto the surface of silica MBs. FIG. 21D shows the surface zeta-potential of silica MBs with different modification.

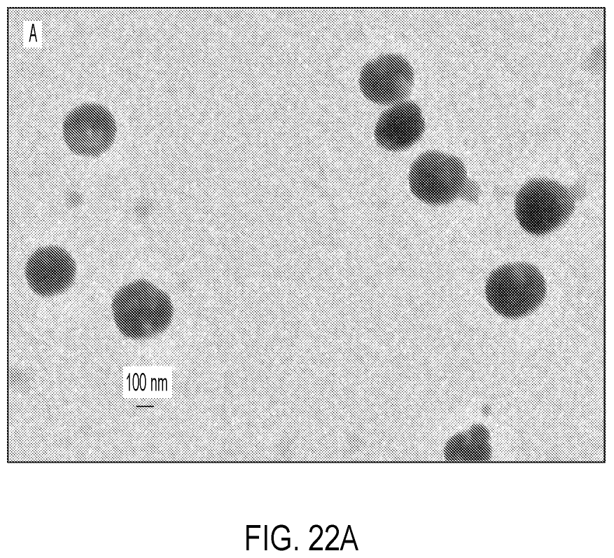

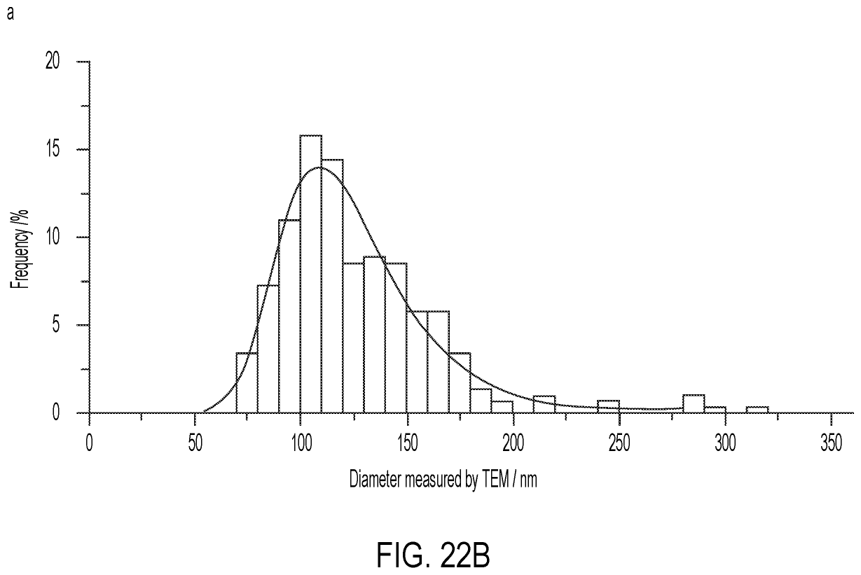

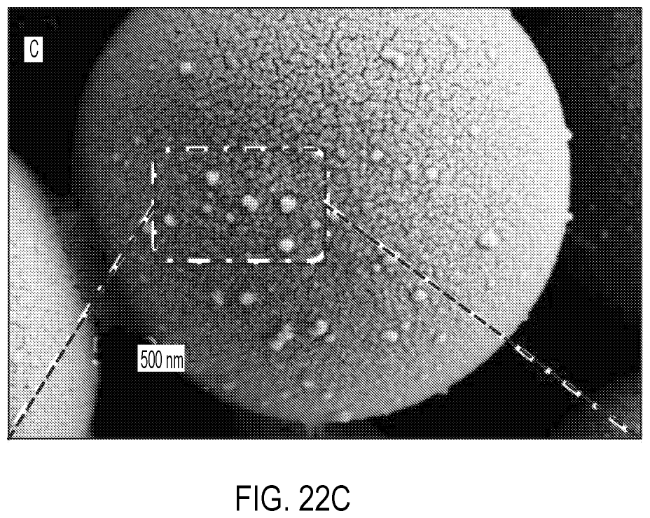

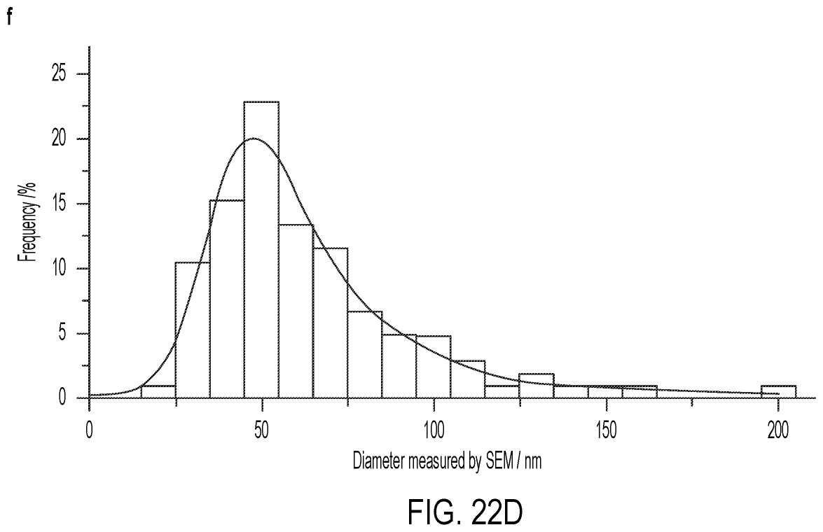

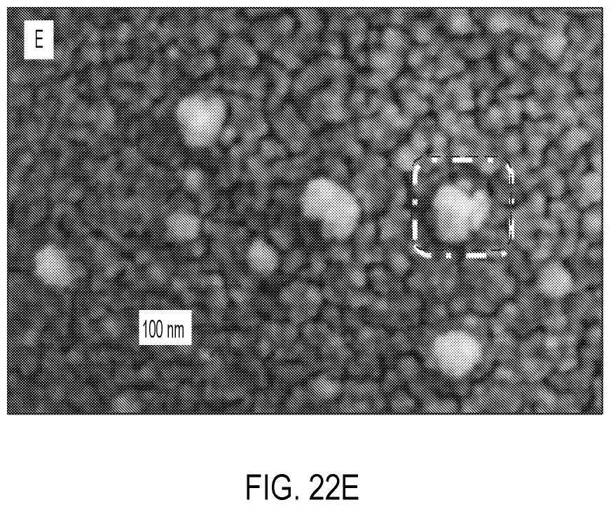

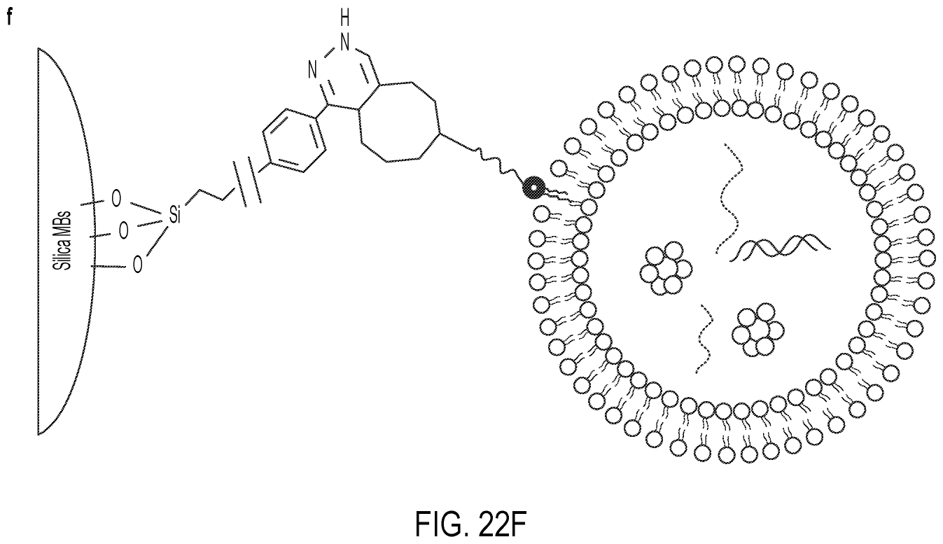

FIGS. 22A-22F are images and graphs showing the characterization of tumor-derived EVs in solution and captured on silica MBs. FIG. 22A is a representative TEM image (scale bar, 100 nm) of A673-derived EVs. FIG. 22B shows size distribution (n=291, 30-400 nm in diameter) of A673-derived EVs, measured by transmission electron microscopy (TEM). FIG. 22C is a representative SEM image (scale bar, 500 nm) of a silica microbead with captured EVs. FIG. 22D shows the size distribution (n=200, 30-200 nm in diameter) of A673-derived EVs captured on silica MBs measured by scanning electron microscopy (SEM). FIG. 22E is a higher magnification of FIG. 12C to show the EVs captured on the nanoroughed surface of silica MBs. FIG. 22F is a graphic illustration depicting how the lipid molecule was used for immobilizing a nEV onto a Tz-modified silica microbead.

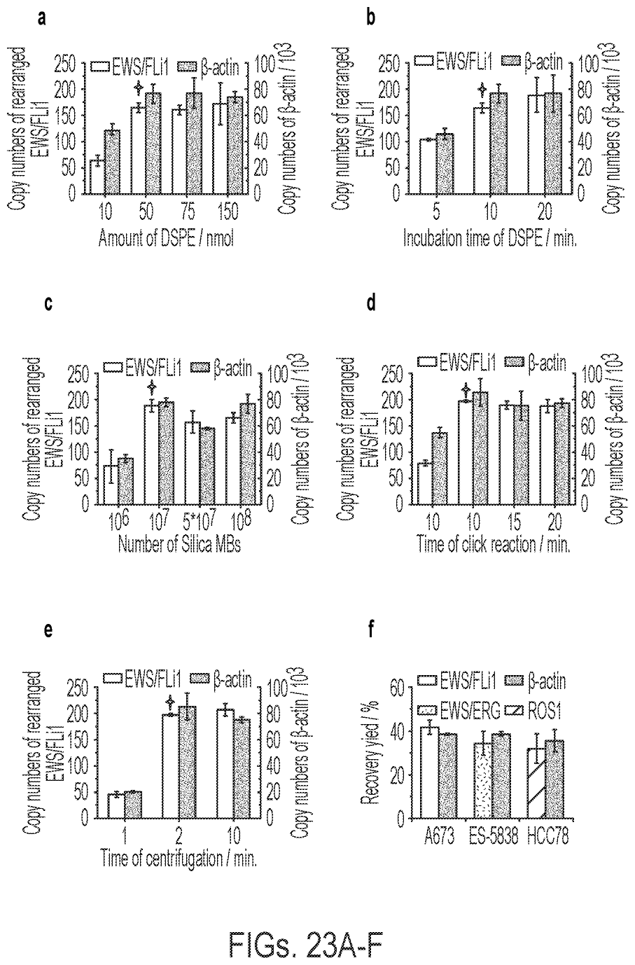

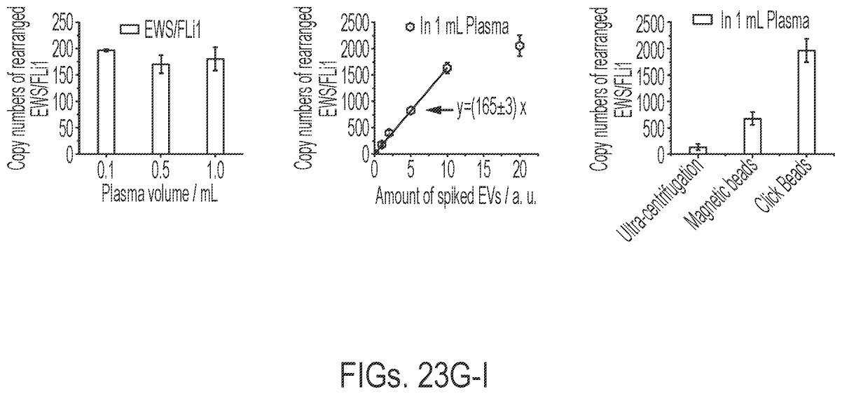

FIGS. 23A-23I are graphs showing optimization of isolation efficiency for total EVs (β-actin) and A673 EVs (EWS/Fli-1) using artificial blood plasma samples. Recovery performance for total EVs and A673 EVs as a function of lipid (DSPE) quantity FIG. 23A, incubation time of the lipid molecules with model plasma samples FIG. 23B), number of Tetrazine modified silica MBs for EVs loading FIG. 23C), time of click chemistry for lipid-labeled EVs and Tz-grafted silica MBs FIG. 23D), time of centrifugation for EVs captured on silica MBs FIG. 23E). FIG. 23F) General applicability of lipid-based label for EV isolation was validated using artificial plasma samples containing EVs of different cell lines, i.e., A673, ES-5838, HCC78. FIG. 23G) Copy numbers of EWS/FLi1 rearrangements observed from different volume of plasma with the same amount of spiked A673 EVs (i.e., 0.1 mL with 10⁷ beads, 0.5 mL with 5*10⁷ beads and 1.0 mL with 10⁸ beads). FIG. 23H) Dynamic ranges of Click Beads for A673-EV isolation from 1 mL plasma samples using 10⁸ silica MBs. Spiked A673 EVs amount ranged from 1 to 20 units of original EV samples. 50 nmol DSPE was used for 1, 2 and 5 unit-spiked plasma samples, 100 nmol DSPE for 10 unit-spiked plasma samples and 200 nmol DSPE for 20 unit-spiked plasma samples. FIG. 23I) Copy numbers of EWS/FLi-1 rearrangements observed for ultracentrifugation, magnetic beads and Click Beads.

### Example 7

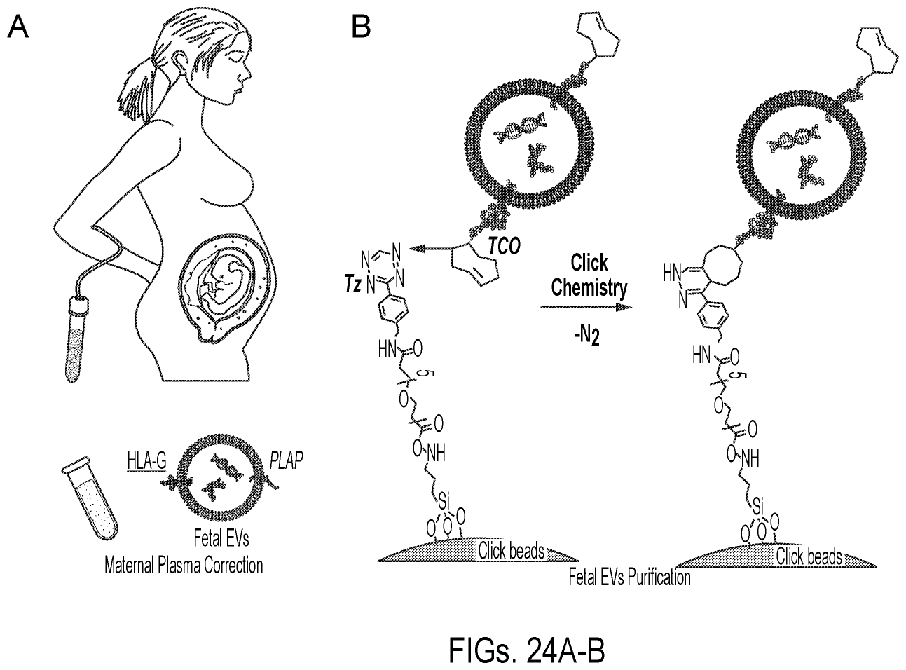

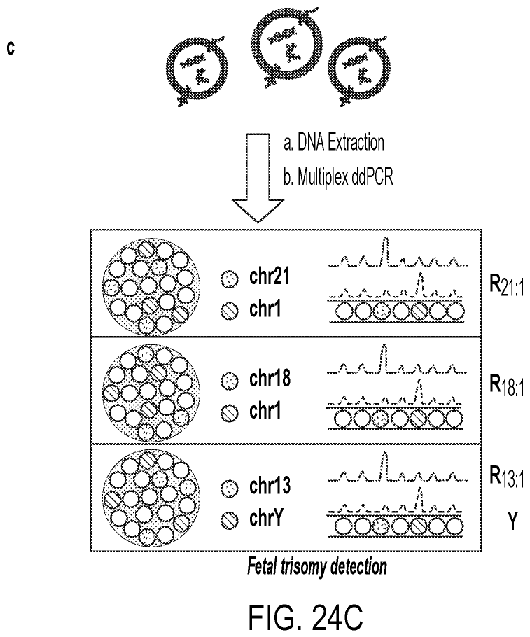

FIGS. 24A-24C are schematics showing a rapid isolation and analysis system for placenta-derived EVs from maternal blood plasma samples. FIG. 24A) Label: Placenta-derived EVs in blood plasma are labeled by a dual-antibody system (anti-HLA-G and anti-PLAP). FIG. 24B) High-efficiency capture: the labeled placenta-derived EVs are captured onto the tetrazine modified silica microbeads (silica MBs) by click chemistry. FIG. 24C) The isolated EVs are then lysed to release placenta-derived EV-derived DNA, which is extracted for downstream analysis by Droplet Digital™ PCR (ddPCR). This workflow was utilized to identify trisomy 21, trisomy 18 and trisomy 13.

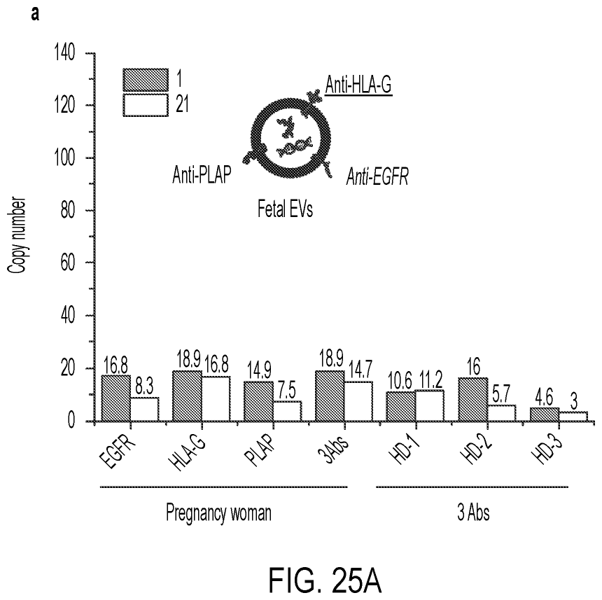

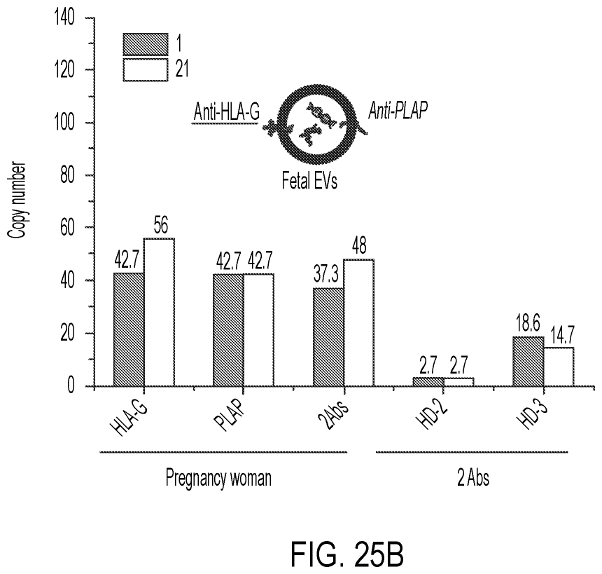

FIGS. 25A and 25B are graphs showing optimization of Click Beads for isolation of placenta-derived EVs using maternal plasma samples and female healthy donor plasma samples. Comparison of placenta-derived EV-DNA (located on chromosome 1 and 21) from pregnancy woman and female healthy donors' plasma samples using three antibodies (anti-PLAP, anti-HLA-G and anti-EGFR) FIG. 25A), and two antibodies (anti-PLAP and anti-HLA-G) FIG. 25B).

1. A method of selectively capturing an extracellular vesicle (EV) from a sample, comprising:
functionalizing a capture agent for said EV with a first molecule from a first bioorthogonal functional group such that said capture agent remains attachable to said EV and said first molecule is also able to bond to a second molecule from a second bioorthogonal functional group, said second molecule being complementary to said first molecule;
mixing said functionalized capture agent with said sample such that said functionalized capture agent binds to said EV and such that an activated sample is formed;
functionalizing a capture surface with said second molecule; and
depositing at least a portion of said activated sample on at least a portion of said functionalized capture surface to thereby selectively capture said EV by binding of said second molecule with said first molecule,
wherein said first molecule from said first bioorthogonal functional group and said capture agent are present in a molar ratio of between 2:1 to 10:1.

2. The method of claim 1, wherein said first molecule from said biorthogonal functional group is selected from the list consisting of trans-cyclooctene (TCO), Alkyne, and a cyclooctyne derivative.

3. The method of claim 2, wherein the cyclooctyne derivative comprises dibenzylcyclooctyne (DBCO) or biarylazacyclooctynone (BARAC).

4. The method of claim 1, wherein said second molecule from said second biorthogonal functional group is selected from the list consisting of tetrazine (TZ) and azide.

5. The method of claim 1, wherein said capture surface comprises a nanostructured surface.

6. The method of claim 1, further comprising functionalizing a second capture agent for said EV with said first molecule such that said second capture agent remains attachable to said EV and said first molecule is also able to bond to said molecule comprising said second molecule from said second bioorthogonal functional group, and
wherein said second capture agent is distinct from said capture agent.

7. A method of assaying for a cancer in a subject comprising:
selectively capturing an extracellular vesicle (EV) from a sample from said subject, said selectively capturing an EV comprising:
functionalizing a capture agent for said EV with a first molecule from a first bioorthogonal functional group such that said capture agent remains attachable to said EV and said first molecule is also able to bond to a second molecule from a second bioorthogonal functional group, said second molecule being complementary to said first molecule;
mixing said functionalized capture agent with said sample such that said functionalized capture agent binds to said EV and such an activated sample is formed;
functionalizing a capture surface with said second molecule; and
depositing at least a portion of said activated sample on at least a portion of said functionalized capture surface to thereby selectively capture said EV by binding of said molecule with said first molecule;
releasing said EV from said capture surface;
assaying a nucleic acid sequence from said EV; and
determining from said assaying of said nucleic acid sequence from said EV whether said cancer is present in said subject,
wherein said first molecule from said first bioorthogonal functional group and said capture agent are present in a molar ratio of between 2:1 to 10:1.

8. The method of claim 7, wherein said first molecule from said biorthogonal functional group is selected from the list consisting of trans-cyclooctene (TCO), Alkyne, and a cyclooctyne derivative.

9. The method of claim 8, wherein the cyclooctyne derivative comprises dibenzylcyclooctyne (DBCO) or biarylazacyclooctynone (BARAC).

10. The method of claim 7, wherein said second molecule from said second biorthogonal functional group is selected from the list consisting of tetrazine (TZ) and azide.

11. The method of claim 7, wherein said capture surface comprises a nanostructured surface.

12. The method of claim 7, further comprising functionalizing a second capture agent for said EV with said first molecule such that said second capture agent remains attachable to said EV and said first molecule is also able to bond to said molecule comprising said second molecule from said second bioorthogonal functional group, and
wherein said second capture agent is distinct from said capture agent.

13. The method of claim 7, wherein said releasing said EV from said capture surface comprises use of a cleaving agent.

14. A method of assaying for a cancer in a subject comprising:
selectively capturing a plurality of extracellular vesicles (EVs) from a sample from said subject, wherein each extracellular vesicle (EV) of said plurality of EVs is selectively captured comprising:
functionalizing a capture agent for an EV of said plurality of EVs with a first molecule from a first bioorthogonal functional group such that said capture agent remains attachable to said EV and said first molecule is also able to bond to a second molecule from a second bioorthogonal functional group, said second molecule being complementary to said first molecule;
mixing said functionalized capture agent with said sample such that said functionalized capture agent binds to said EV and such an activated sample is formed;
functionalizing a capture surface with said second molecule; and
depositing at least a portion of said activated sample on at least a portion of said functionalized capture surface to thereby selectively capture said EV by binding of said molecule with said first molecule;
releasing said plurality of EVs from said capture surface;
assaying a plurality of nucleic acid sequences from said plurality of EVs;
creating an expression profile of said plurality of nucleic acid sequences, said expression profile comprising a quantification of each of said plurality of nucleic acid sequences;
comparing said expression profile with a control; and
determining from said comparing of said expression profile with said control whether said cancer is present in said subject,
wherein said first molecule from said first bioorthogonal functional group and said capture agent are present in a molar ratio of between 2:1 to 10:1.

15. A kit for selectively capturing an extracellular vesicle (EV) from a sample, comprising:
a capture agent comprising a first molecule from a first bioorthogonal functional group;
a substrate comprising a functionalized capture surface having a second molecule from a second bioorthogonal functional group, said second molecule being complementary to said first molecule;
a cleaving agent;
instructions for mixing said capture agent with said sample such that said capture agent binds to said EV and such that an activated sample is formed;
instructions for depositing at least a portion of said activated sample on at least a portion of said functionalized capture surface to thereby selectively capture said EV by binding of said second molecule with said first molecule; and
instructions for using said cleaving agent to release said EV from said capture surface,
wherein said first molecule from said first bioorthogonal functional group and said capture agent are present in a molar ratio of between 2:1 to 10:1.

16. The kit of claim 15, wherein said first molecule from said biorthogonal functional group is selected from the list consisting of trans-cyclooctene (TCO), Alkyne, and a cyclooctyne derivative.

17. The kit of claim 16, wherein the cyclooctyne derivative comprises dibenzylcyclooctyne (DBCO) or biarylazacyclooctynone (BARAC).

18. The kit of claim 15, wherein said second molecule from said second biorthogonal functional group is selected from the list consisting of tetrazine (TZ) and azide.

19. The kit of claim 15, wherein said capture surface comprises a nanostructured surface.

20. The kit of claim 15 further comprising a plurality of reagents for a nucleic acid test.
